(19) United States
(12) Patent Application Publication
(10) Pub. No.: US 2018/0282780 A1
(43) Pub. Date: Oct. 4, 2018

(54) SUSCEPTIBILITY AND RESISTANCE OF MICROORGANISMS

(71) Applicant: Specific Technologies, LLC, West Palm Beach, FL (US)

(72) Inventors: Pragya Singh, Saratoga, CA (US); Raymond Anthony Martino, Los Gatos, CA (US); Paul A. Rhodes, Woodside, CA (US); Richard Shu-Chung Huang, Palo Alto, CA (US); Sung Hyun Lim, Mountain View, CA (US)

(73) Assignee: Specific Technologies, LLC, West Palm Beach, FL (US)

(21) Appl. No.: 15/939,736

(22) Filed: Mar. 29, 2018

Related U.S. Application Data
(60) Provisional application No. 62/478,458, filed on Mar. 29, 2017.

(51) Int. Cl. C12Q 1/18

(52) U.S. Cl. CPC C12Q 1/18; G01N 2415/00

(57) ABSTRACT

Devices, systems, and methods for species and/or strain specific identification and assessment of susceptibility of microorganisms based on the response of sensors in a colorimetric sensor array to metabolic products of the microorganism. An exemplary method according to an embodiment of the present disclosure can include culturing a sample that contains microorganisms. The sample can be in a medium which is exposed to a colorimetric sensor array. A test substance can be introduced to the sample. The method can assess a susceptibility of the microorganisms to the test substance based on a change in at least one sensor in the colorimetric sensor array. Sensors in the colorimetric sensor array can change in response to volatile organic compounds produced by the microorganisms after addition of the test substance.

### CROSS REFERENCE TO RELATED APPLICATION

This application claims priority to U.S. Patent Application No. 62/478,458, filed Mar. 29, 2017, titled “Susceptibility and Resistance of Microorganisms,” the contents of which are incorporated herein by reference.

### FIELD

This disclosure is related to determining antibiotic susceptibility or resistance of microorganisms.

### BACKGROUND

Before Penicillin, minor injury came at risk of death. With the invention of Penicillin, the average human lifespan increased by almost 20 years. As the use of these medicines prompted the evolution of bacteria resistant to them, two new generations of antibiotics were invented. However, there are now bacteria resistant to all three generations of antibiotics, and a new generation has not been found. Thus antibiotic resistance has now become a global threat for populations in both the developed and developing world. In the U.S., the Center for Disease Control and Prevention (CDC) has recently estimated 2 million patients per year are directly affected by antibiotic-resistant pathogens, leading to more than 23,000 deaths. In addition, the health care costs related to antibiotic resistance are steadily increasing, already exceeding costs of more than $20 billion annually; with the inclusion of lost productivity the total cost exceeds $35 billion, and these enormous numbers are predicted to rapidly grow. The indiscriminate use of broad-spectrum generic antibiotics, particularly in the developing world, continues to drive the evolution of drug-resistant bacteria, creating a global health crisis. The O'Neill Report, issued in May 2016 has raised awareness of this problem, and leading to an unprecedented meeting at the United Nations of the heads of state of over 100 countries on Sep. 21, 2016. This report declared the need for new systems to perform antibiotic susceptibility testing (AST), rapidly and at low cost. Despite the increasing concerns, currently available methods are time-consuming and generally require colonies to be grown on plates followed by 8-15 hours of further testing. Conventional antimicrobial susceptibility testing (AST) include disk diffusion, agar dilution, antibiotic gradient disks, and broth microdilution testing, which is the current reference standard.

Sepsis is initially diagnosed from clinical signs and symptoms such as otherwise unexplained body temperature alterations (hyperthermia or hypothermia), tachycardia, tachypnea, peripheral vasodilation, or shock. The clinical diagnosis typically triggers the immediate use of broad-spectrum antibiotic treatment while the two-day process of AST is performed. However, with the rise of antibiotic-resistant infection, the broad-spectrum prophylactic increasingly fails. Without effective antibiotics, blood infection is the deadliest human condition: inappropriate antibiotic therapy doubles sepsis-induced mortality, which has been reported to increase 7% every hour from the onset of septic shock until the delivery of an effective antibiotic. Clearly, with drug resistant blood infections, patient survival depends upon a far faster AST than the current 2-day methods.

One of the earliest antimicrobial susceptibility testing methods was the macrobroth or tube-dilution method. This procedure involved preparing two-fold dilutions of antibiotics (eg, 1, 2, 4, 8, and 16 μg/mL) in a liquid growth medium dispensed in test tubes. The antibiotic-containing tubes were inoculated with a standardized bacterial suspension of 1-5×105 CFU/mL. Following overnight incubation at 35° C., the tubes were examined for visible bacterial growth as evidenced by turbidity. The lowest concentration of antibiotic that prevented growth represented the minimal inhibitory concentration (MIC). The precision of this method was considered to be plus or minus 1 two-fold concentration, due in large part to the practice of manually preparing serial dilutions of the antibiotics. The advantage of this technique was the generation of a quantitative result (ie, the MIC). The principal disadvantages of the macrodilution method were the tedious, manual task of preparing the antibiotic solutions for each test, the possibility of errors in preparation of the antibiotic solutions, and the relatively large amount of reagents and space required for each test.

Presently, semi-automated instrument systems are utilized to standardize the reading of end points and produce susceptibility test results. For example, some instruments can incubate and analyze 40-96 microdilution trays. As in the manual method, the instruments monitor the wells for turbidity changes to indicate the presence or absence of bacteria.

### SUMMARY

One aspect includes determining a susceptibility of a microorganism to an antibiotic by culturing the microorganism with the antibiotic in the presence of a colorimetric sensor array, thereby exposing sensors in the colorimetric sensor array to volatile organic compounds produced by the microorganisms. The response of the colorimetric sensor may indicate whether the microorganism is susceptible to the applied antibiotic at the specific concentration.

However, in the United States, alarming trends in resistance are now also reported for a number of Gram-negative pathogens. For example, extended-spectrum beta-lactamase (ESBL) organisms are now endemic in many ICUs, and 15 to 20% of all Pseudomonas aeruginosa isolates from serious infections are categorized as multidrug resistant (MDR) because of reduced in vitro susceptibility to three or more classes of antibiotics. Of even more concern are pathogens for which clinicians have few antibiotic options, namely Acinetobacter baumanii and carbepenemase-producing Enterobacteriaceae (CPE). In the case of these Gram-negative organisms, studies also point to an association between resistance and both clinical and economic outcomes.

In some aspects, a multiplicity of containers (wells), each containing growth medium and a concentration of antibiotic or without antibiotic (control, e.g., no antibiotic) and each in gaseous contact with a colorimetric sensors array, may be utilized to determine a susceptibility of a given microorganism, or a sample suspected to contain a microorganism susceptible to an antibiotic. In some embodiments, the multiplicity of wells may include a variety of antibiotics, with each antibiotic possibly being applied at different concentrations. The aggregate colorimetric sensor array response from the combination of sensors in the multiplicity of wells may indicate the identity of a microorganism in the sample and the susceptibility of the microorganism to the applied antibiotics at the applied concentrations. In some aspects, this system could be utilized to identify a type of bacteria or other microorganism that has infected (for example a patient) by culturing a blood sample from the patient or other mammal in the presence of the colorimetric sensor array. Accordingly, if a patient is suspected to have sepsis, samples of the patient's blood could be tested to determine (1) if the patient has an infection, (2) the identity of the microorganism infecting the patient, and (3) the susceptibility or resistance of that microorganism to the applied antibiotics.

A general aspect includes culturing a sample including a microorganism in the presence of a colorimetric sensor array, thereby exposing sensors in the colorimetric sensor array to volatile organic compounds produced by the microorganism, identifying the microorganism by species and/or strain based on the response of the sensors in the colorimetric sensor array to the volatile organic compounds produced by the microorganism, and assessing susceptibility of the microorganism to a substance based on the response of the sensors in the colorimetric sensor array to the volatile organic compounds produced by the microorganism.

Another general aspect is related to reducing a population of a selected microorganism in a mammal carrying the microorganism, and includes collecting a sample including at least one of the selected microorganisms from the mammal, culturing the microorganism(s) in the presence of a colorimetric sensor array, thereby exposing sensors in the colorimetric sensor array to volatile organic compounds produced by the microorganism(s), identifying susceptibility of the microorganism(s) to a substance based on the response of the sensors in the colorimetric sensor array to the volatile organic compounds produced by the microorganism(s), and administering a dose of the substance to the mammal, wherein the dose is effective to reduce the population of the identified microorganism in the mammal.

A third general aspect is related to reducing a bacterial population in a mammal showing symptoms of infection, and includes collecting a sample of bacteria from the mammal, culturing some of the bacteria in the presence of a colorimetric sensor array, thereby exposing sensors in the colorimetric sensor array to volatile organic compounds produced by the bacteria, identifying susceptibility of the bacteria to a substance based on the response of the sensors in the colorimetric sensor array to the volatile organic compounds produced by the bacteria, and administering a dose of the substance to the mammal, wherein the dose is effective to reduce the number of the identified bacteria in the mammal.

A fourth general aspect includes culturing a sample comprising a species of bacteria in the presence of a colorimetric sensor array, thereby exposing sensors in the colorimetric sensor array to volatile organic compounds produced by the bacteria, and identifying the bacteria by species and/or strain based on the response of the sensors in the colorimetric sensor array to the volatile organic compounds produced by the bacteria, wherein identifying the bacteria by species and/or strain comprises identifying a substance-resistant strain of a species of bacteria.

Implementations of the general aspects may include one or more of the following features.

The microorganism may be identified by species and/or strain (e.g., based on the response of the sensors in the colorimetric sensor array to the volatile organic compounds produced by the bacteria) before identifying the susceptibility of the bacteria to the substance. Identifying the bacteria by species and/or strain may include identifying an antibiotic-resistant mutant.

The microorganism may be collected from a substrate before culturing the microorganism. The substrate may be, for example, woven or nonwoven fabric, paper, metal, or plastic.

In some cases, the microorganism is collected from a mammal (e.g., a human) before culturing the microorganism. Collecting the microorganism from the mammal may include collecting a fluid sample or a tissue, including swabs, sample from the mammal, wherein the fluid sample comprises a liquid (e.g., blood), or a combination thereof. The mammal may be showing symptoms of bacteremia.

A substance to which the microorganism is susceptible may be identified based on the response of the sensors in the colorimetric sensor array to the volatile organic compounds produced by the microorganism. The substance may be, for example, a medication approved for use in animals or humans. The substance may be selected based on the identified species and/or strain of the microorganism (e.g., bacteria). In some cases, a dose of the substance is administered to the mammal from which the microorganism was collected, wherein the dose is effective to reduce the number of the identified microorganisms in the mammal.

In some cases, susceptibility of the microorganism to the substance may be assessed within 64 hours, within 48 hours, within 36 hours, within 24 hours, within 12 hours, within 10 hours, within 8 hours, within 4 hours, or within 2 hours after identification of the microorganism.

In certain cases, culturing the bacteria includes culturing the bacteria on a solid medium or in a liquid medium. The response of each sensor may include a change in one or more color components of the sensor. The temporal and/or static response of the sensors may yield a temporal or static color response pattern of the bacteria. Identifying the bacteria by species and/or strain may include comparing the temporal and/or static color pattern of the bacteria with a library of temporal and/or static color response patterns characteristic of known strains of bacteria.

Susceptibility or resistance of a bacteria or other microorganism to a substance may be assessed based on the response of the sensors in the colorimetric sensor array to the volatile organic compounds produced by the bacteria. A dose of a substance to which the bacteria is susceptible may be administered to the mammal from which the bacteria was collected, the dose being effective to reduce the number of the identified bacteria in the mammal.

Advantages described herein include species identification and susceptibility assay to be complete less than 24 hours after samples reach the laboratory.

### BRIEF DESCRIPTION OF DRAWINGS

FIG. 1 depicts a colorimetric sensor array.

FIG. 2A shows a colorimetric sensor array before exposure to metabolic products of E. coli 25922. FIG. 2B shows the colorimetric sensor array of FIG. 2A after exposure to E. coli 25922 on growth medium for five hours. FIG. 2C shows the difference between the colorimetric sensor arrays of FIGS. 2A and 2B.

FIGS. 3A-3D show the temporal response of four different sensors in the colorimetric sensor array shown in FIGS. 2A-2C.

FIG. 4 depicts a container including a colorimetric sensor array.

FIGS. 5A-D show temporal results for various sensors of a colorimetric sensor array used for identification of bacteria.

FIGS. 6A-D show temporal results for various sensors of a colorimetric sensor array used for strain-specific identification of bacteria.

FIG. 7 depicts an apparatus for automatic identification of microorganisms and/or assessing a susceptibility or resistance of microorganism.

FIG. 8 depicts an apparatus for assessing antibiotic susceptibility of a microorganism.

FIGS. 9A-B show temporal results of susceptibility tests for various sensors of a colorimetric sensor array for identified strains of K. pneumoniae.

FIGS. 10A-C show temporal results of susceptibility tests for various sensors of a colorimetric sensor array for identified strains of S. aureus.

FIGS. 11A-B show temporal results of susceptibility tests for various sensors of a colorimetric sensor array for identified strains of K. pneumoniae.

FIGS. 12A-C show temporal results of susceptibility tests for various sensors of a colorimetric sensor array for identified strains of S. aureus.

FIGS. 13A-23B show temporal results of susceptibility tests for various sensors of a colorimetric sensor array for identified strains of E. faecium.

FIGS. 24A-31B show temporal results of susceptibility tests for various sensors of a colorimetric sensor array for identified strains of K. pneumoniae.

FIGS. 32A-39B show temporal results of susceptibility tests for various sensors of a colorimetric sensor array for identified strains of P. aeruginosa.

FIGS. 40A-47 show temporal results of susceptibility tests for various sensors of a colorimetric sensor array for identified strains of S. aureus.

FIG. 48 shows graphs of temporal results of a spectral component of an indicator of a colorimetric sensor array after addition of an antibiotic.

FIG. 49 shows graphs of temporal results of a spectral component of an indicator of a colorimetric sensor array after addition of an antibiotic.

FIG. 50 shows a graph of temporal results of a spectral component of an indicator of a colorimetric sensor array after addition of an antibiotic.

FIG. 51 shows a graph of temporal results of a spectral component of an indicator of a colorimetric sensor array after addition of an antibiotic.

FIG. 52 shows graphs of temporal results of a spectral component of an indicator of a colorimetric sensor array after addition of an antibiotic.

FIG. 53 shows a graph of temporal results of a spectral component of an indicator of a colorimetric sensor array after addition of an antibiotic.

FIG. 54 illustrates a perspective view of an example of a container configured for use to assess a susceptibility or resistance of microorganisms.

FIG. 55 illustrates a flow chart showing an example method of determining a susceptibility from a perturbation in VOCs after addition of an antibiotic.

### DETAILED DESCRIPTION

A colorimetric sensor is a sensor that includes one or more materials that undergo a change in spectral properties upon exposure to an appropriate change in the environment of the sensor. The change in spectral properties may include a change in the absorbance, fluorescence, and/or phosphorescence of electromagnetic radiation, including ultraviolet, visible, and/or infrared radiation. Culturing a sample including a microorganism (e.g., a species of bacteria) in the presence of a colorimetric sensor array exposes sensors in the colorimetric sensor array to compounds produced by the microorganism. U.S. Patent Publication No. 2008/0199904 to Suslick et al., U.S. Patent Publication No. 2010/0166604 to Lim et al., and Carey et al., “Rapid Identification of Bacteria with a Disposable Colorimetric Sensing Array,” J. Am. Chem. Soc. 2011, 133, 7571-7576, all of which are incorporated by reference herein, describe identification of bacteria from volatiles they produce using colorimetric sensor arrays. Response of the sensors in the colorimetric sensor array to the compounds yields a species and/or strain-specific temporal or static color response pattern, allowing the microorganism to be identified by comparison of the color response pattern with color response patterns for known strains. Comparison may be achieved, for example, visually or automatically. In some examples, the compounds will be volatile organic compounds (“VOCs”) produced by the microorganism. These VOCs may be detected in the gaseous state after they off gas from a liquid sample or may be sensed by detecting them while still in solution in a liquid sample. In other examples, the compounds may be compounds excreted by the microorganism that are not VOCs that remain in the liquid phase in a liquid sample.

While bacteria of a given species share certain characteristics, different strains of the same species yield noticeably different color response patterns (or “fingerprints”), allowing discrimination between strains of the given species (e.g., between Staphylococcus aureus and methicillin-resistant Staphylococcus aureus and between Enterococus faecalis and vancomycin-resistant Enterococus faecalis). The color response patterns allow identification of microorganisms by species and/or strain and certain antibiotic resistant characteristics in a fraction of the time (e.g., about three-quarters of the time, about one-half of the time, or about one-quarter of the time) of other methods, based at least in part on conditions such as concentration, culture medium, culture conditions (e.g., temperature), and the like.

In addition, colorimetric sensor arrays can be also used to assess susceptibility of a microorganism (e.g., a microorganism identified based on the response of the sensors in a first colorimetric sensor array to the volatile organic compounds produced by the microorganism) to a substance, such as a drug approved for human use. This can be achieved by culturing the microorganism with various concentrations of a substance, and monitoring the color response patterns. If the microorganism is fully or partially susceptible, the color response pattern will be decreased or almost non-existent. In some cases, susceptibility can be assessed in a matter of hours (e.g., less than twelve hours, less than ten hours, less than 8 hours, or less than 6 hours, or less than 4 hours) after identification of the microorganism. This sequence of identification and assessment of susceptibility allows rapid treatment of patients experiencing a malady (e.g., sepsis, meningitis, etc.) related to a pathogenic microorganism. In some cases, susceptibility or resistance of a microorganism is assessed without prior identification of the microorganism.

In other embodiments, a mode of resistance of a microorganism may be assessed by the signature of the reduced response to the colorimetric sensor array when it is cultured in the presence of a substance. The reduced response may be in terms of rate of growth, overall intensity of response, and the specific signature of the response. In some embodiments, known modes of resistances may have certain signatures that may be utilized to identify a mode of resistance of a microorganism. In some embodiments, this may even be determined before the microorganism is identified. That way, certain classes of antibiotics could be eliminated, or a caregiver could apply a cocktail of antibiotics with an educated guess of the infection at an earlier stage. A library or dataset of average responses for certain classes of known modes of antibiotic resistance may be provided. That way, the colorimetric sensor array 100 response of a given microorganism to a given antibiotic at an applied concentration can be compared to the responses of a library of data that contains averages or examples of responses of microorganisms with known susceptibility or resistance modes.

Microorganisms such as bacteria, yeasts, protozoa, and fungi can be identified as described herein. Species of bacteria that can be identified include, for example, Staphylococcus aureus, Staphylococcus epidermidis, Staphylococcus sciuri, Pseudomonas aeruginosa, Enterococcus faecium, Enterococcus faecalis, Escherichia coli, Klebsiella pneumoniae, Streptococcus pneumoniae, Streptococcus pyrogenes, Vibrio cholera, Achromobacter xylosoxidans, Burkholderia cepacia, Citrobacter diversus, Citrobacter freundii, Micrococcus leuteus, Proteus mirabilis, Proteus vulgaris, Staphylococcus lugdunegis, Salmonella typhi, Streptococcus Group A, Streptococcus Group B, S. marcescens, Enterobacter cloacae, Bacillis anthracis, Bordetella pertussis, Clostridium sp., Clostridium botulinum, Clostridium tetani, Corynebacterium diphtheria, Moraxalla (Brauhamella) catarrhalis, Shigella spp., Haemophilus influenza, Stenotrophomonas maltophili, Pseudomonas perolens, Pseuomonas fragi, Bacteroides fragilis, Fusobacterium sp. Veillonella sp., Yersinia pestis, and Yersinia pseudotuberculosis. Strains of bacteria that can be identified include, for example, S. aureus 25923, S. aureus 29213, S. aureus 43300, S. aureus IS-13, S. aureus IS-38, S. aureus IS-43, S. aureus IS-70, S. aureus IS-120, S. aureus IS-123, S. aureus IS-124, methicillin-resistant S. aureus 33591, S. epidermidis 35984, S. sciuri 49575, P. aeruginosa 10145, P. aeruginosa IS-15, P. aeruginosa IS-65, P. aeruginosa IS-22, P. aeruginosa IS-36, P. aeruginosa 27853, E. faecium 19434, E. faecalis 23241, vancoymcin-resistant E. faecalis 51299, E. coli 25922, E. coli 53502, E. coli 35218, E. coli 760728, E. coli IS-39, E. coli IS-44, A. xylosoxidans IS-30, A. xylosoxidans IS-35, A. xylosoxidans IS-46, A. xylosoxidans IS-55, C. diversus IS-01, C. diversus IS-28, C. diversus IS-31, C. diversus IS-33, K. pneumoniae IS-130, K. pneumoniae IS-133, K. pneumoniae IS-136, K. pneumoniae 33495, B. anthrax Ames, B. anthrax UM23CL2, B. anthrax Vollum, Y. pestis C092, Y. pestis Java 9, S. epidermis 12228, S. epidermis IS-60, S. epidermis IS-61, P. miribilis IS-18, P. miribilis IS-19, P. miribilis 12453, S. marcescens IS-48, S. marcescens IS-05, and S. marcescens 13880, where “IS-#” refers to clinical isolates and the other strains are ATCC® reference strains. Species of fungi that can be identified include, for example, Microsporum sp. Trichophyton sp. Epidermophyton sp., Sporothrix schenckii, Wangiella dermatitidis, Pseudallescheria boydii, Madurella grisea, Histoplasma capsulatum, Blastomyces dermatitidis, Coccidioides immitis, Cryptococcus neoformans, Aspergillus fumigatus, Aspergillus niger, and Candida albicans. Similarly, yeasts including Ascomycota (Saccharomycotina, Taphyrinomycotina, Schizosaccharomycetes) and Basidiomycota (Agaricomycotina, Tremellomycetes, Pucciniomycotina, Microbotryomycetes) can be identified and, if desired, assessed for susceptibility. Examples include Saccharomyces cerevisiae and Candida albicans. Protozoa including flagellates (e.g., Giardia lamnblia), amoeboids (e.g., Entamoeba histolytica), sporozoans (e.g., Plasmodium knowlesi), and ciliates (e.g., Balantidium coli) may also be identified as described herein.

Colorimetric sensor arrays described herein can be used to identify and/or monitor pathogenic and non-pathogenic microorganisms. In one example, a sample including microorganisms from a mammal (e.g., a human) showing symptoms a malady or in need of treatment for a malady can be taken from the mammal (e.g., in the form of a fluid sample such as blood or exhaled breath, or in the form of a tissue sample) and cultured in the presence of a colorimetric sensor array. In other examples, microorganisms such as Saccharomyces cerevisiae and others can be monitored in processes such as baking and alcoholic fermentation processes, electricity generation in microbial fuel cells, and biofuel production.

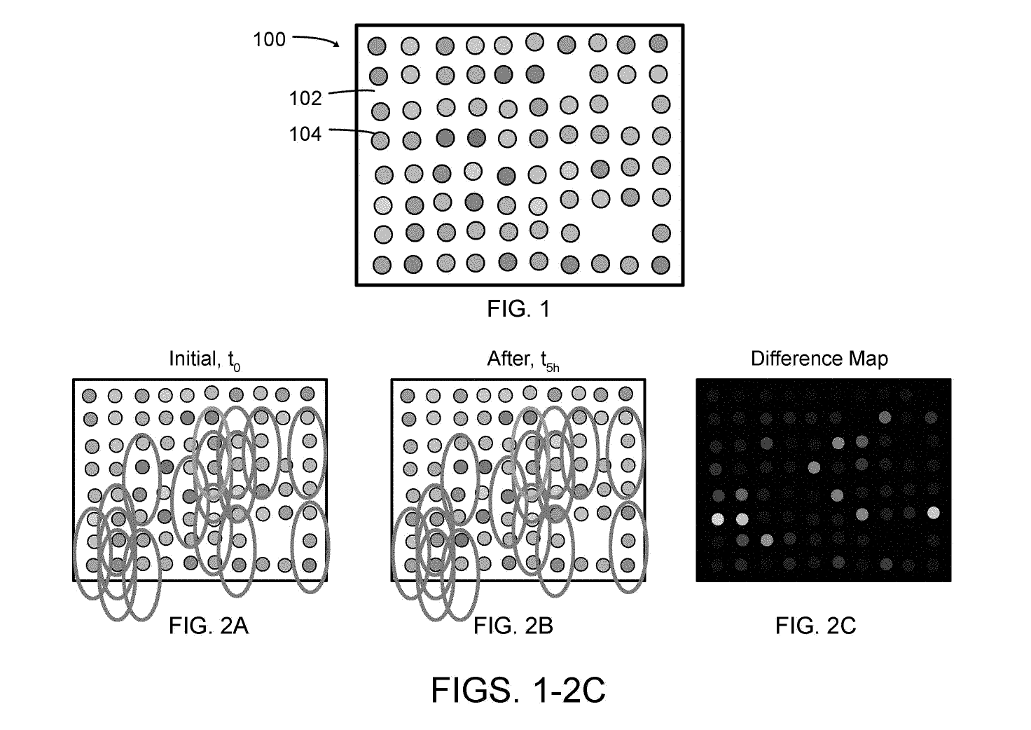

FIG. 1 depicts an exemplary colorimetric sensor array 100. Colorimetric sensor array 100 includes a substrate 102 having a multiplicity of colorimetric sensors 104, each colorimetric sensor including an indicator selected to change color in the presence of at least one volatile organic compound. Colorimetric sensor arrays typically include an array of chemoresponsive colorants, where the colors of the chemoresponsive colorant are affected by a wide range of analyte-dye interactions. “Chemoresponsive colorant” refers to any material that absorbs, reflects, and/or emits light when exposed to higher frequency electromagnetic radiation. A light-absorbing portion of a chemical indicator is referred to as a chromophore, and a light-emitting portion of a colorant is referred to as a fluorophore. “Chemoresponsive colorant” generally refers to an indicator that undergoes a change in spectral properties in response to an appropriate change in its chemical environment. “Change in spectral properties” generally refers to a change in the frequency and/or intensity of the light the colorant absorbs and/or emits. Chemoresponsive colorants include dyes and pigments.

Examples of chemoresponsive dyes include Lewis acid-base dyes, metalloporphyrins, free base porphyrins, phthalocyanines, pH sensitive dyes, solvatochromic dyes, vapochromic dyes, redox sensitive dyes, and metal ion sensitive dyes. Chemoresponsive dyes may be responsive to one or more chemical interactions including Lewis acid-base interaction, Brønsted acid-base interaction, ligand binding, π-π complexation, hydrogen bonding, polarization, oxidation/reduction, and metal coordination.

The chemoresponsive dye may be, for example, a Lewis acid-base dye, such as a Lewis acid dye or a Lewis base dye. A Lewis acid-base dye is a dye that can interact with a substance by acceptor-donor sharing of a pair of electrons with the substance, resulting in a change in spectral properties. The change in spectral properties for a Lewis acid-base dye may be related to Lewis acid-base interaction and ligand binding, but also to π-π complexation, hydrogen bonding, and/or polarity changes. Lewis acid-base dyes include metal-ion containing dyes, such as metalloporphyrins and other metal ion ligating macrocycles or chelating dyes; boron- and boronic acid containing dyes; and dyes with accessible heteroatoms (e.g., N, O, S, P) with lone electron pairs capable of Lewis coordination (e.g., “complexometric dyes”).

Examples of Lewis acid-base dyes include metal ion-containing dyes, such as metal ion-containing porphyrins (i.e., metalloporphyrins), salen complexes, chlorins, bispocket porphyrins, and phthalocyanines. Diversity within the metalloporphyrins can be obtained by variation of the parent porphyrin, the porphyrin metal center, or the peripheral porphyrin substituents. The parent porphyrin is also referred to as a free-base porphyrin, which has two central nitrogen atoms protonated (i.e., hydrogen cations bonded to two of the central pyrrole nitrogen atoms). In one example, a parent porphyrin is the so-called free base form 5,10,15,20-tetraphenylporphyrin (H₂TPP), its dianion is 5,10,15,20-tetraphenyl-porphyrinate(-2) (TPP dianion), its metalated complexes, and its acid forms (H₃TPP⁺ and H₄TPP+²). This porphyrin may form metalated complexes, for example, with Sn⁴⁺, Co³⁺, Co²⁺, Cr³⁺, Mn³⁺, Fe³⁺, Cu²⁺, Ru²⁺, Zn²⁺, Ag²⁺, In³⁺, and Ir³⁺. Metal ion-containing metalloporphyrin dyes are described, for example, in U.S. Pat. No. 6,368,558 to Suslick et al. and in U.S. Patent Application Publication No. 2003/0143112 to Suslick et al., both of which are incorporated by reference herein.

Visible spectral shifts and absorption intensity differences for metalloporphyrins may occur upon ligation of the metal center, leading to readily observable changes in spectral properties. The magnitude of this spectral shift typically correlates with the polarizability of the ligand, thus allowing for distinction between analytes based on the electronic properties of the analytes. Using metal centers that span a range of chemical hardness and ligand binding affinity, it may be possible to differentiate between a wide range of volatile analytes, including molecules having soft functional groups such as thiols, and molecules having hard functional groups such as amines. Because porphyrins can exhibit wavelength and intensity changes in their absorption bands with varying solvent polarity, an array that includes porphyrins may be used to colorimetrically distinguish among a series of weakly coordinating solvent vapors, such as arenes, halocarbons, and ketones.

The chemoresponsive dye may be, for example, a structure-sensitive porphyrin. Structure-sensitive porphyrins include modified porphyrins that include a super structure bonded to the periphery of the porphyrin. For example, metalloporphyrins functionalized with a super structure at the periphery may limit steric access to the metal ion, allowing for shape-selective distinction of analytes, such as between n-hexylamine and cyclohexylamine. Controlling the ligation of various nitrogenous ligands to dendrimer-metalloporphyrins can provide for selectivities over a range of more than 10⁴.

Examples of super structures that may be bonded to a porphyrin include dendrimers, siloxyl groups, aryl groups such as phenyl groups, alkyl groups such as t-butyl groups, organometallic groups, inorganic groups, and other bulky substituents. Porphyrins bearing super structures may be selective to molecular shape, including sensitivity to steric factors, enantiomeric factors, and regioisomeric factors. For example, the structures may provide sterically constrained pockets on one or both faces of the porphyrin. Porphyrins bearing super structures also may be sensitive to factors such as hydrogen bonding and acid-base functionalities. Metal ion-containing metalloporphyrin dyes that include a super structure bonded to the periphery of the porphyrin, and methods of making such dyes, are disclosed, for example, in U.S. Pat. No. 6,495,102 to Suslick et al., which is incorporated by reference herein.

One example of modified porphyrins that include a super structure bonded to the periphery of the porphyrins is the family of tetrakis(2,4,6-trimethoxyphenyl)-porphyrin (TTMPP). By varying the metal in this porphyrin, it is possible to distinguish between substances such as between t-butylamine and n-butylamine, and between cyclohexylamine and n-hexylamine. Another example of a modified porphyrin that includes a super structure bonded to the periphery of the porphyrin is the family of silylether-metalloporphyrins. For example, scaffolds derived from the reaction of 5,10,15,20-tetrakis(2′,6′-dihydroxyphenyl)-porphyrinatozinc(II) with t-butyldimethylsilyl chloride provide Zn(II) porphyrin having in which the two faces are protected with six, seven, or eight siloxyl groups. This can result in a set of three porphyrins having similar electronic properties, but having different hindrance around the central metal atom present in the porphyrin. The shape selectivities of these porphyrins may be up to 10⁷ or greater.

Other examples of modified porphyrins that include a super structure bonded to the periphery of the porphyrin include siloxyl-substituted bis-pocket porphyrins, such as 5-phenyl-10,15,20-tris(2′,6′-dihydroxyphenyl)porphyrinatozinc(II); 5,10,15,20-tetrakis(2′,6′-dihydroxyphenyl)porphyrinatozinc(II); 5(phenyl)-10,15,20-trikis(2′,6′-disilyloxyphenyl)porphyrinatozinc(II); 5,10,15-trikis(2′,6′-disilyloxyphenyl)-20-(2′-hydroxy-6′-silyloxyphenyl)p-orphyrinatozinc(II). The shape selectivities of these porphyrins may be up to 10⁷ or greater compared to unhindered metalloporphyrins. Fine-tuning of ligation properties of these porphyrins may be possible, such as by using pockets of varying steric demands.

Other examples of metal ion-containing metalloporphyrin dyes that include a super structure bonded to the periphery of the porphyrin include 2,3,7,8,12,13,17,18-octafluoro-5,10,15,20-tetrakis(pentafluorophenyl)-porphyrinatocobalt(II); 2,3,7,8,12,13,17,18-octabromo-5,10,15,20-tetraphenylporphyrinatozinc (II); 5,10,15,20-tetraphenylporphyrinatozinc(II); 5(phenyl)-10,15,20-trikis(2′,6′-bis(dimethyl-t-butylsiloxyl)phenyl)porphyrinatozinc(II); 5,10,15,20-tetrakis(2′,6′-bis(dimethyl-t-butylsiloxyl)phenyl)porphyrinatozinc(II); 5,10,15,20-tetraphenylporphyrinatocobalt (II); 5,10,15,20-tetrakis(2,6-difluorophenyl)-porphyrinatozinc(II); and 5,10,15,20-tetrakis(2,4,6-trimethylphenyl)-porphyrinatozinc(II).

An array that includes a structure-sensitive porphyrin may be used in combinatorial libraries for shape selective detection of substrates. Such an array also may include a structure-sensitive having chiral super structures on the periphery of the porphyrin, which may provide for identification of chiral substrates, such as drugs, natural products and components of biological samples from a patient. Such an array also may be used for analysis of biological entities based on the surface proteins, oligosaccharides, antigens, etc., that interact with the metalloporphyrins. Examples of biological entities include individual species of bacteria and viruses. Such an array also may be used for analysis of nucleic acid sequences, including specific recognition of individual sequences of nucleic acids. Substituents on the porphyrins that would be particularly useful in this regard include known DNA intercalating molecules and nucleic acid oligomers.

The chemoresponsive dye may be, for example, a pH sensitive dye. Dyes that are pH sensitive include pH indicator or acid-base indicator dyes that may change color upon exposure to acids or bases. Examples of pH sensitive dyes include Brønsted acid dyes. A Brønsted acid dye is a proton donor that can donate a proton to a Brønsted base (i.e., a proton acceptor), resulting in a change in spectral properties. Under certain pH conditions, a Brønsted acid dye may be a Brønsted base.

Examples of Brønsted acid dyes include protonated, but non-metalated, porphyrins; chlorines; bispocket porphyrins; phthalocyanines; and related polypyrrolic dyes. Examples of non-metalated porphyrin Brønsted acid dyes include 5,10,15,20-tetrakis(2′,6′-bis(dimethyl-t-butyl siloxyl)phenyl)porphyrin dication; 5,10,15,20-tetraphenyl-21H,23H-porphyrin; or 5,10,15,20-tetraphenylporphyrin dication. Other examples of Brønsted acid dyes include Chlorophenol Red, Bromocresol Green, Bromocresol Purple, Bromothymol Blue, Bromopyrogallol Red, Pyrocatechol Violet, Phenol Red, Thymol Blue, Cresol Red, Alizarin, Mordant Orange, Methyl Orange, Methyl Red, Congo Red, Victoria Blue B, Eosin Blue, Fat Brown B, Benzopurpurin 4B, Phloxine B, Orange G, Metanil Yellow, Naphthol Green B, Methylene Blue, Safranine O, Methylene Violet 3RAX, Sudan Orange G, Morin Hydrate, Neutral Red, Disperse Orange #25, Rosolic Acid, Fat Brown RR, Cyanidin chloride, 3,6-Acridineamine, 6′-Butoxy-2,6-diamino-3,3′-azodipyridine, para-Rosaniline Base, Acridine Orange Base, Crystal Violet, Malachite Green Carbinol Base, Nile Red, Nile Blue, Nitrazine Yellow, Bromophenol Red, Bromophenol Blue, Bromoxylenol Blue, Xylenol Orange Tetrasodium Salt, 1-[4-[[4-(dimethylamino)phenyl]azo]phenyl]-2,2,2-trifluoro-ethanone-, 4-[2-[4-(dimethylamino)phenyl]ethenyl]-2,6-dimethyl-pyrylium perchlorate, and 1-amino-4-(4-decylphenylazo)-naphthalene.

The chemoresponsive dye may be, for example, a solvatochromic dye or a vapochromic dye. Solvatochromic dyes may change color depending upon the local polarity of their liquid micro-environment. Vapochromic dyes may change color depending upon the local polarity of their gaseous micro-environment. Most dyes are solvatochromic and/or vapochromic to some extent; however, some are much more responsive than others, especially those that can have strong dipole-dipole interactions. Examples of solvatochromic dyes include Reichardt's dyes, Nile Red, Fluorescein, and polypyrrolic dyes.

An array that includes a pH sensitive dye and/or a solvatochromic or vapochromic dye may be useful in differentiating analytes that do not bind to, or bind only weakly to, metal ions. Such analytes include acidic compounds, such as carboxylic acids, and certain organic compounds lacking ligatable functionality. Examples of organic compounds lacking ligatable functionality include simple alkanes, arenes, and some alkenes and alkynes, especially if sterically hindered. Examples of organic compounds lacking ligatable functionality also include molecules that are sufficiently sterically hindered to preclude effective ligation. Arrays that include a pH sensitive and/or a solvatochromic or vapochromic dye are described, for example, in U.S. Patent Application Publication No. 2003/0143112 to Suslick et al., which is incorporated by reference herein.

The chemoresponsive dye may be, for example, a redox sensitive dye that undergoes a change in spectral properties depending upon its oxidation state. Examples of dyes that are redox sensitive include redox indicators such as methylene blue, naphthol blue-black, brilliant ponceau, .alpha.-naphthoflavone, basic fuchsin, quinoline yellow, thionin acetate, methyl orange, neutral red, diphenylamine, diphenylaminesulfonic acid, 1,10-phenanthroline iron(II), permanganate salts, silver salts, and mercuric salts.

The chemoresponsive dye may be, for example, a metal ion sensitive dye that undergoes a change in spectral properties in the presence of metal ions. Examples of dyes that are metal ion sensitive include metal ion indicator dyes such as eriochrome black T, murexide, 1-(2-pyridylazo)-2naphthol, and pyrocatechol violet.

The chemoresponsive colorant may be a chemoresponsive pigment. In some cases, the chemoresponsive pigment is a porous pigment. A porous pigment particle has a chemoresponsive surface area that is much greater than the chemoresponsive surface area of a corresponding nonporous pigment particle. Examples of porous pigments include porous calcium carbonate, porous magnesium carbonate, porous silica, porous alumina, porous titania, and zeolites.

The chemoresponsive colorant may be a chemoresponsive nanoparticle. A chemoresponsive nanoparticle may be a discrete nanoparticle, or it may be formed from nanoparticle-forming ions or molecules. The nanoparticle may be in a variety of forms, including a nanosphere, a nanorod, a nanofiber, and a nanotube. Examples of chemoresponsive nanoparticles include nanoporous porphyrin solids, semiconductor nanoparticles such as quantum dots, and metal nanoparticles.

The use of more than one type of chemoresponsive colorant may expand the range of analytes to which the array is sensitive, may improve sensitivity to some analytes, and/or may increase the ability to discriminate between analytes. In some cases, a colorimetric array includes 2 to 1,000 sensors, 4 to 500 sensors, or 8 to 250 sensors. In certain cases, a colorimetric array includes from 10 to 100 sensors (e.g., 16 to 80 sensors, 36 sensors, or 60 sensors). Each sensor in a colorimetric array may include a different colorant. However, it may be desirable to include duplicate sensors that include the same colorant. Duplicate sensors may be useful, for example, to provide redundancy to the array and/or to serve as an indicator for quality control. Table 1 lists exemplary chemoresponsive colorants for a colorimetric sensor array having 36 sensors.

TABLE 1
Exemplary chemoresponsive colorants for a colorimetric sensor array.

| No. | Colorant |
|---|---|
| 1 | 5,10,15,20-Tetraphenyl-21H,23H-porphine zinc |
| 2 | 5,10,15,20-Tetraphenyl-21H,23H-porphine copper(II) |
| 3 | 5,10,15,20-Tetraphenyl-21H,23H-porphine manganese(III) chloride |
| 4 | 2,3,7,8,12,13,17,18-Octaethyl-21H,23H-porphine iron(III) chloride |
| 5 | 5,10,15,20-Tetraphenyl-21H,23H-porphine cobalt(II) |
| 6 | meso-Tetra(2,4,6-trimethylphenyl)porphine |
| 7 | Nitrazine Yellow (basic) |
| 8 | Methyl Red (basic) |
| 9 | Chlorophenol Red (basic) |
| 10 | Napthyl Blue Black |
| 11 | Bromothymol Blue (basic) |
| 12 | Thymol Blue (basic) |
| 13 | m-Cresol purple (basic) |
| 14 | Zinc (II) Acetate with m-Cresol purple (basic) |
| 15 | Mercury (II) Chloride with Bromophenol Blue (basic) |
| 16 | Mercury (II) Chloride with Bromocresol Green (basic) |
| 17 | Lead (II) Acetate |
| 18 | Tetraiodophenolsulfonephthalein |
| 19 | Fluorescein |
| 20 | Bromocresol Green |
| 21 | Methyl Red |
| 22 | Bromocresol Purple |
| 23 | Bromophenol Red |
| 24 | Brilliant Yellow |
| 25 | Silver nitrate + Bromophenol Blue (basic) |
| 26 | Silver nitrate + Bromocresol Green (basic) |
| 27 | Cresol Red (acidic) |
| 28 | Disperse Orange 25 |
| 29 | m-Cresol Purple |
| 30 | Nitrazine Yellow |
| 31 | Cresol Red |
| 32 | Bromocresol Green |
| 33 | Phenol Red |
| 34 | Thymol Blue |
| 35 | Bromophenol Blue |
| 36 | m-Cresol Purple |

For gas or vapor analytes, a gas stream containing the analyte is passed over the array, and images may be obtained before, during and/or after exposure to the gas stream. Preferably, an image is obtained after the sample and the array have equilibrated. If the gas stream is not pressurized, it may be useful to use a miniaturized pump.

For analytes dissolved in a solvent, either aqueous or non-aqueous, the first image may be obtained in air or, preferably, after exposure to the pure carrier solvent that is used of the sample. The second image of the array may be obtained after the start of the exposure of the array to the sample. Preferably an image is obtained after the sample and the array have equilibrated.

Analyzing the differences between the first image and the second image may include quantitative comparison of the digital images before and after exposure to the analyte. Using customized software or standard graphics software such as Adobe® PhotoShop®, a difference map can be obtained by subtracting the first image from the second image. To avoid subtraction artifacts at the periphery of the spots, the center of each spot can be averaged.

FIGS. 2A-2C are images from a colorimetric sensor array, showing the array before exposure to E. coli 25922 (FIG. 2A), after exposure to E. coli 25922 (FIG. 2B), and a difference map of these two images (FIG. 2C). The comparison data obtained from the difference map includes changes in red, green and blue values (ΔRGB) for each spot in the array. The changes in spectral properties that occur upon exposure to an analyte, and the resultant color difference map, can serve as a unique fingerprint for any analyte or mixture of analytes at a given concentration.

In the simplest case, an analyte can be represented by a single 3x vector representing the ΔRGB values for each colorant, where x is the number of colorants as set forth in equation (1) below. This assumes that equilibration is relatively rapid and that any irreversible reactions between analyte and colorant are slow relative to the initial equilibration time

Difference vector=ΔR1,ΔG1,ΔB1,ΔR2,ΔG2,ΔB2, . . . ΔRx,ΔGx,ΔBx (1)

Alternatively, the temporal response of the analyte can be used to make rapid identification, preferably using a “time-stack vector” of ΔRGB values as a function of time. In equation 2, a time-stack vector is shown for an array of 36 colorants at times m, n, and finally z, all using the initial scan as the baseline for the differences in red, green and blue values:

Time stack vector=ΔR1m,ΔG1m,ΔB1m,ΔR2m,ΔG2m,ΔB2m,−ΔR36m,ΔG36m,ΔB36m, . . . ΔR1n,ΔG1n,ΔB1n, . . . ΔR36m,ΔG36m,ΔB36m, . . . ΔR36z,ΔG36z,ΔB36z (2)

Accordingly, each analyte response can be represented digitally as a vector of dimension 3xz, where x is the number of colorants and z is the number of scans at different times. Quantitative comparison of such difference vectors can be made simply by measuring the Euclidean distance in the 3xz space. Such vectors may then be treated by using chemometric or statistical analyses, including principal component analysis (PCA), hierarchical cluster analysis (HCA) and linear discriminant analysis. Statistical methods suitable for high dimensionality data are preferred. As an example, HCA systematically examines the distance between the vectors that represent each colorant, forming clusters on the basis of the multivariate distances between the analyte responses in the multidimensional ΔRGB color space using the minimum variance (“Ward's”) method for classification. A dendrogram can then be generated that shows the clustering of the data from the Euclidean distances between and among the analyte vectors, much like an ancestral tree.

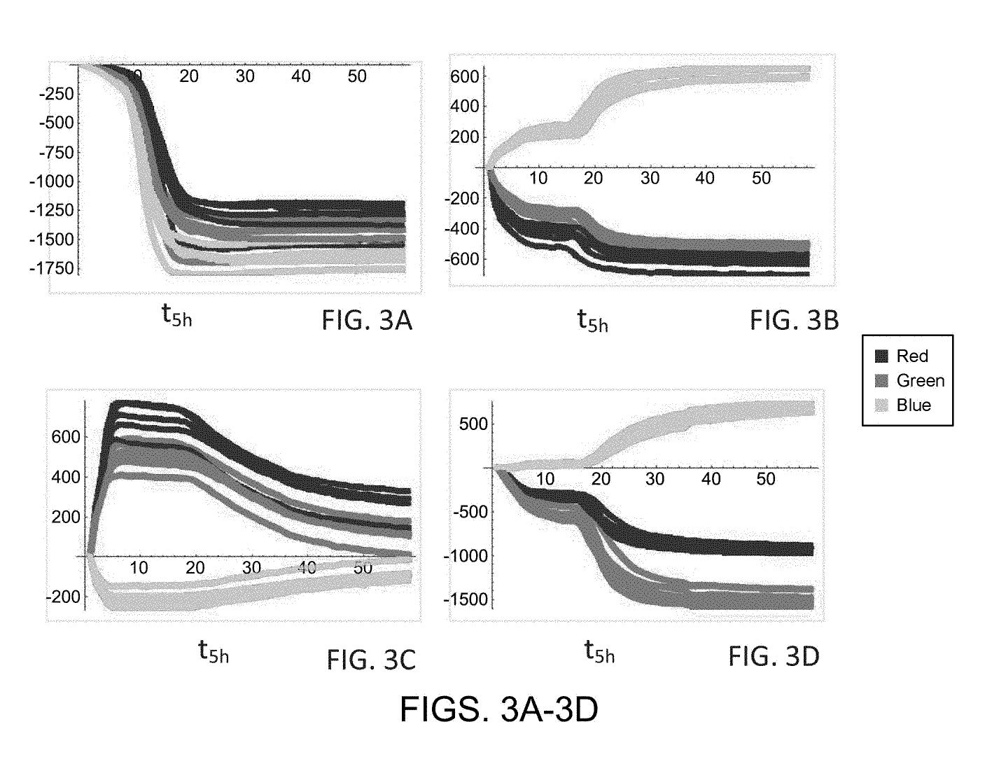

FIGS. 3A-D show the temporal response of four different sensors from the sensor array shown in FIGS. 2A-2C to metabolic products of E. coli 25922. The sample is identified as containing E. coli 25922 by comparison of the temporal responses of the same sensors to a library of responses from known microorganisms.

A colorimetric array may be used to detect analytes in exhaled breath. Detection of compounds in exhaled breath can be useful in detecting infection or disease. The colorimetric detection of ammonia in exhaled breath is described, for example, in U.S. Patent Application Publication No. 2005/0171449 to Suslick et al., which is incorporated by reference herein.

To detect and identify a microorganism by species and/or strain, a sample including the microorganism is placed in a container including culture medium and a colorimetric array, and the temporal response of the sensors to the metabolic products of the microorganism is monitored. Susceptibility can be assessed by inoculating a growth medium including a substance (e.g., an antibiotic) with a microorganism and monitoring the response of the sensors while also monitoring the response of a control (e.g., no antibiotic). If the response does not show growth or growth below a given threshold of the microorganism, the microorganism may be determined to be susceptible to the applied substance.

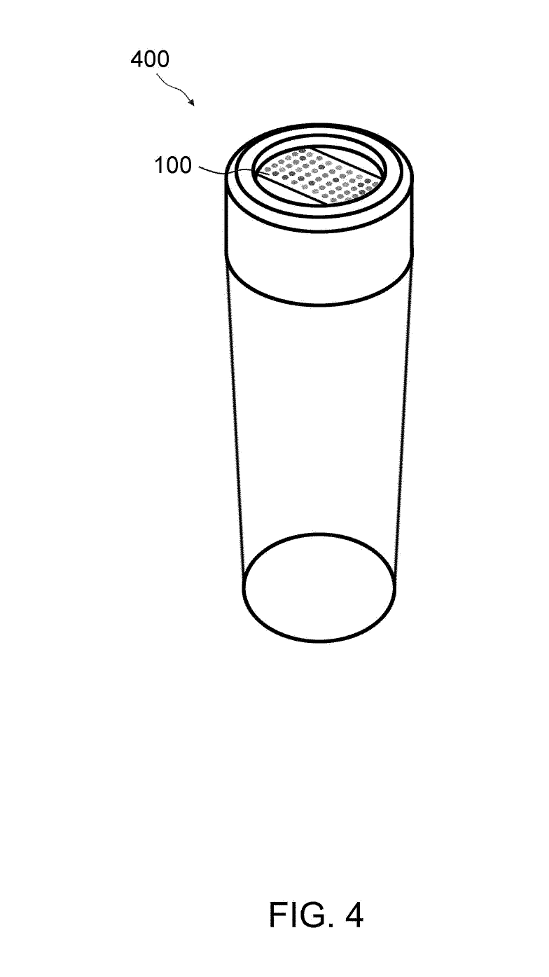

FIG. 4 depicts exemplary container 400 with colorimetric sensor array 100 for detecting detect a microorganism or its susceptibility. Container 400 may include a solid or liquid culture medium generally known in the art. A sample, such as a fluid sample (e.g., blood, sputum, exhaled breath) from a mammal, a tissue sample from a mammal, or the like, is placed or injected in container 400. The colorimetric sensor array 100 may be in gaseous or liquid communication with a fluid sample and/or a solid or liquid culture medium, or other materials containing the sample. This will allow volatile organic compounds or other compounds emitted from the microorganisms to evaporate into the air in the container 400 and come into contact with the colorimetric sensor array 100. In other embodiments, the sample may be in liquid communication with the colorimetric sensor array 100 and therefore the colorimetric sensor array 100 may be exposed to compounds in solution. In some embodiments, container 400 is sealed, and colorimetric sensor array 100 is exposed to volatile organic compounds emitted from the microorganisms during growth. In other embodiments, different containers or other mechanisms could be utilized to expose the colorimetric sensor array 100 to gas emitted from the sample. This could include various channels or tubing that could transport the volatile organic compounds emitted from the sample into a gaseous state.

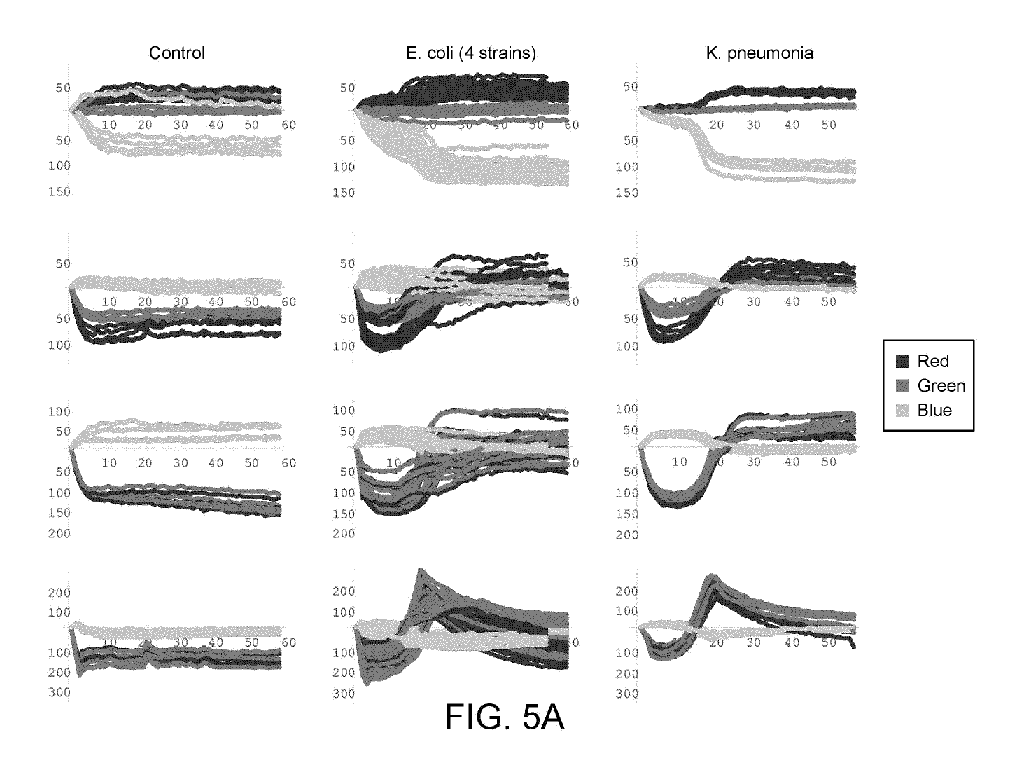

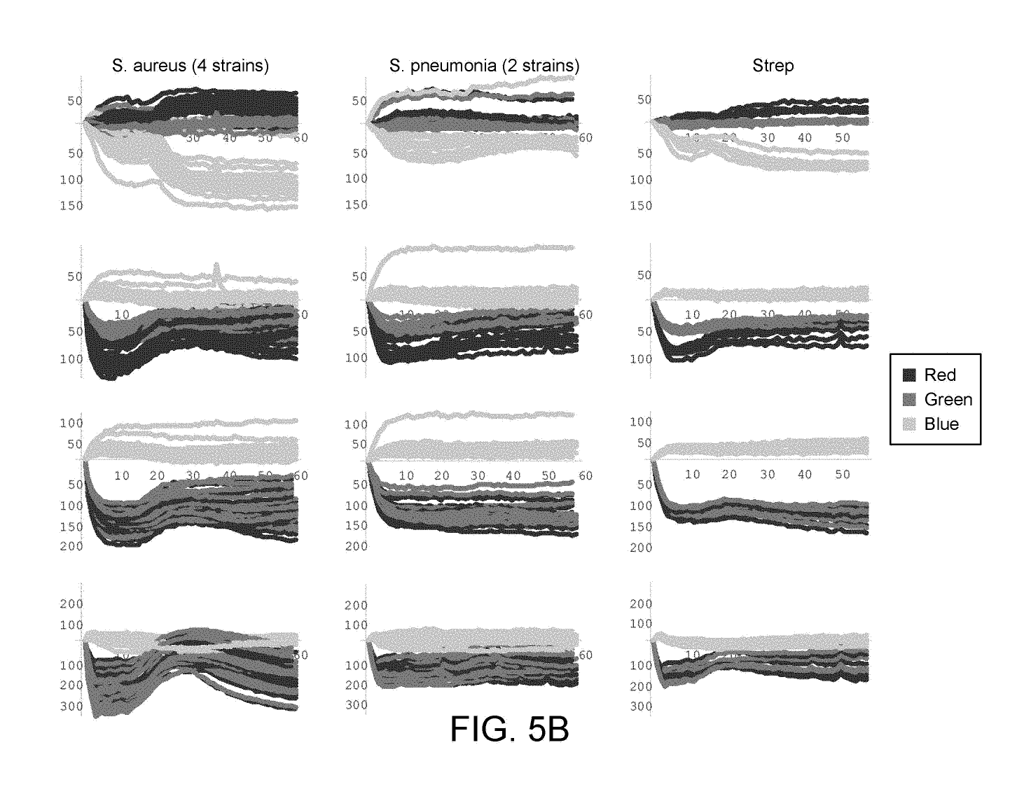

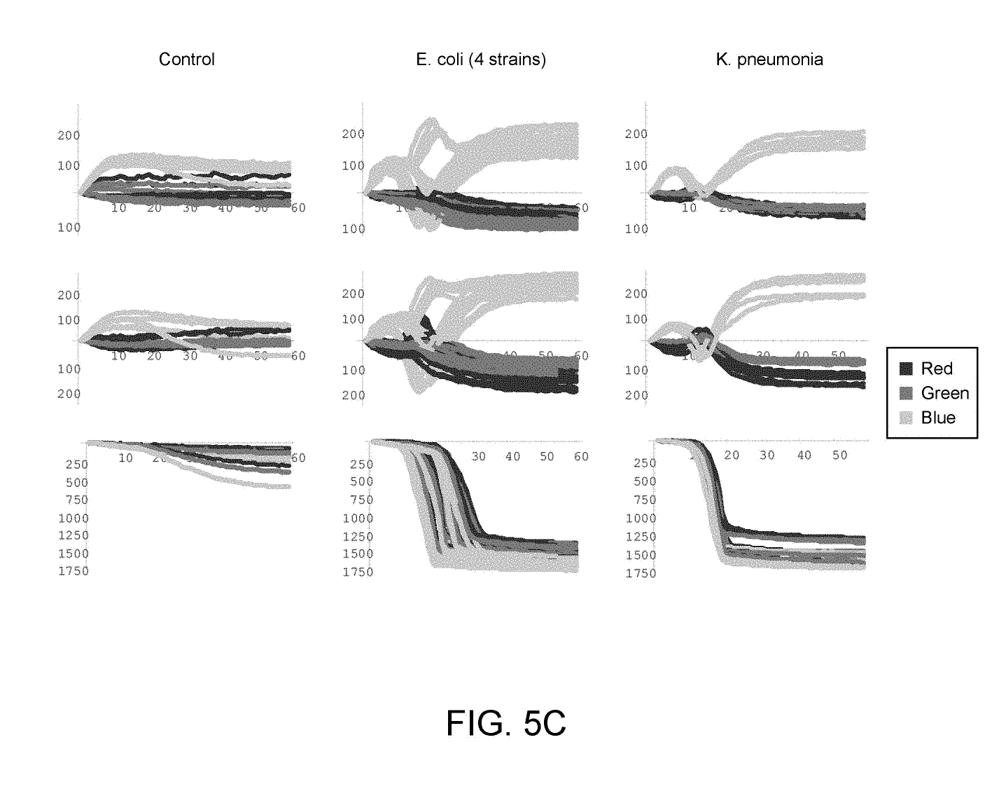

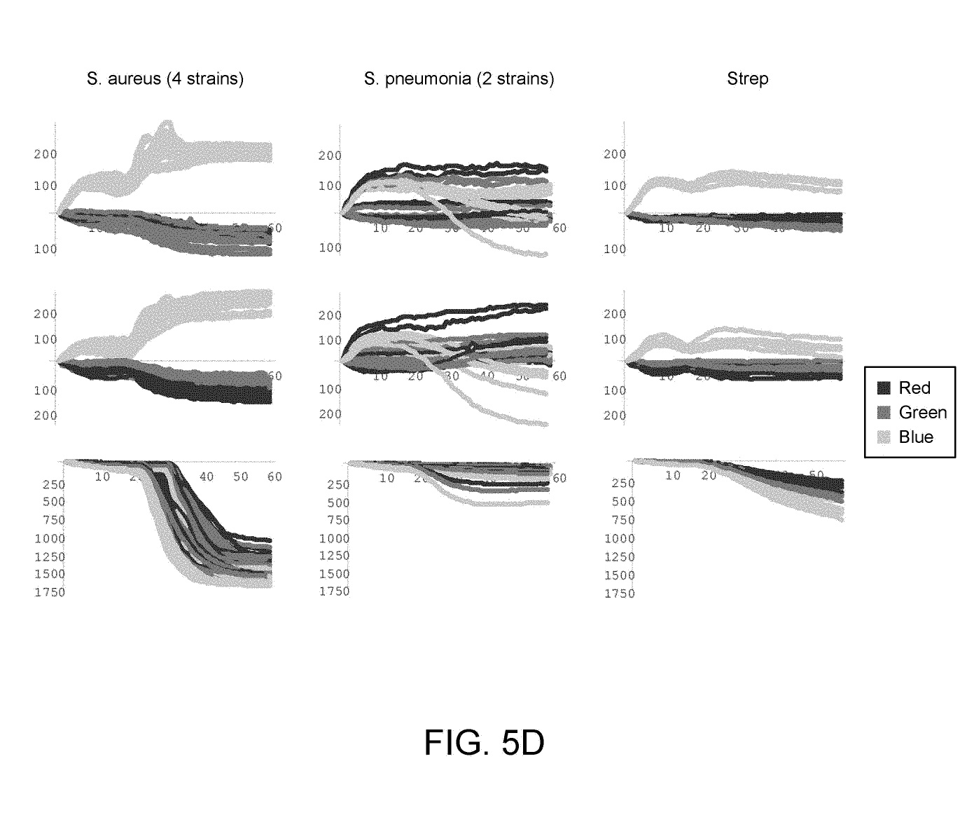

Identification of species and/or strain of the microorganism is achieved by comparison of kinetic profiles of the color sensors in a colorimetric sensor array exposed to metabolic products of the microorganism. For illustration purposes, FIGS. 5A-D show temporal responses of various bacteria for sensors corresponding to those in Table 1, with magnitude of response on the y-axis and time on the x-axis. Based on low/high inoculum concentration, E. coli was identified in 3-6 hours, K. pneumoniae was identified in 3-5 hours, S. aureus was identified in 3-7 hours, S. pneumoniae was identified in 7-9 hours, and Streptococcus Group A and B was identified in 6-9 hours. Blood culture results show an overall identification accuracy of 99% for various species, including S. aureus (18/19 correct), E. faecalis (4/5 correct), E. faecium (6/6 correct), E. coli (15/15 correct), P. mirabilis (4/4 correct), S. marcescens (5/5 correct), E. cloacae (5/5 correct), K. pneumoniae (17/17 correct), P. aeruginosa (17/17 correct), and blood only (8/8 correct). Table 2 shows accuracy of 99% for identification of various bacterial species.

TABLE 2
Identification of Bacterial Species

| Species | Correct/total | Percent correct |
|---|---|---|
| A. xylosioxidans | 24/24 | 100 |
| B. cepacia | 11/12 | 92 |
| C. diversus | 24/24 | 100 |
| C. Freundii | 17/18 | 94 |
| E. coli | 114/114 | 100 |
| K. pneumonia | 18/18 | 100 |
| M. luteus | 18/18 | 100 |
| P. aeruginosa | 24/24 | 100 |
| P. mirabilis | 24/24 | 100 |
| P. vulgaris | 11/12 | 92 |
| S. aureus | 59/60 | 98 |
| S. epidermidis | 18/18 | 100 |
| S. lugdunesis | 18/18 | 100 |
| S. typhi | 12/12 | 100 |
| Control | 6/6 | 100 |

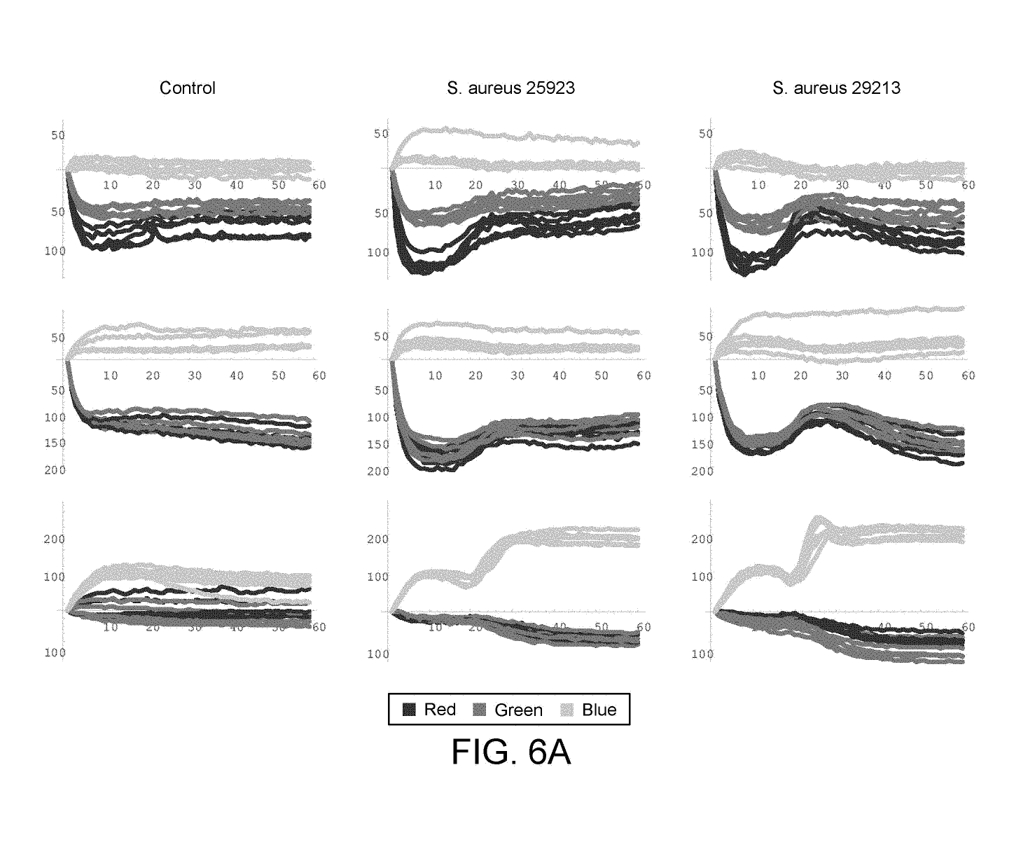

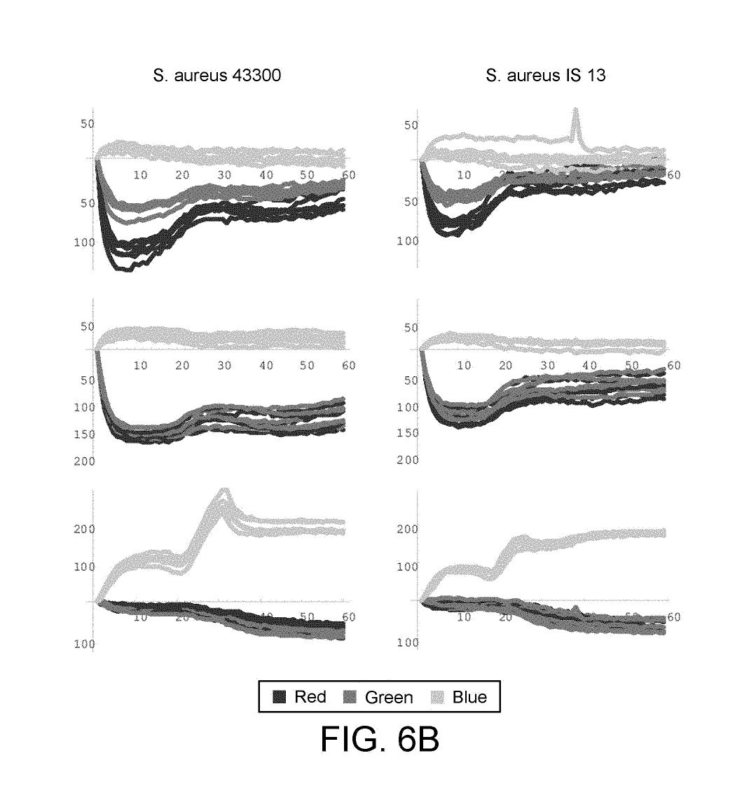

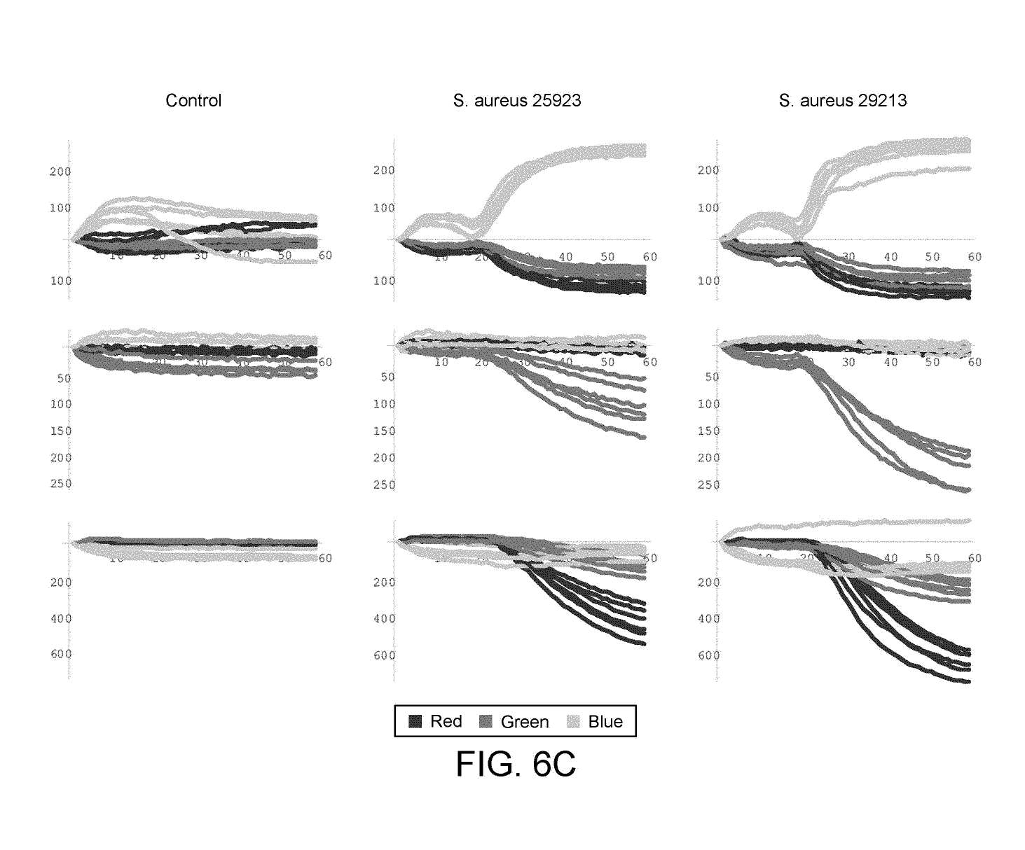

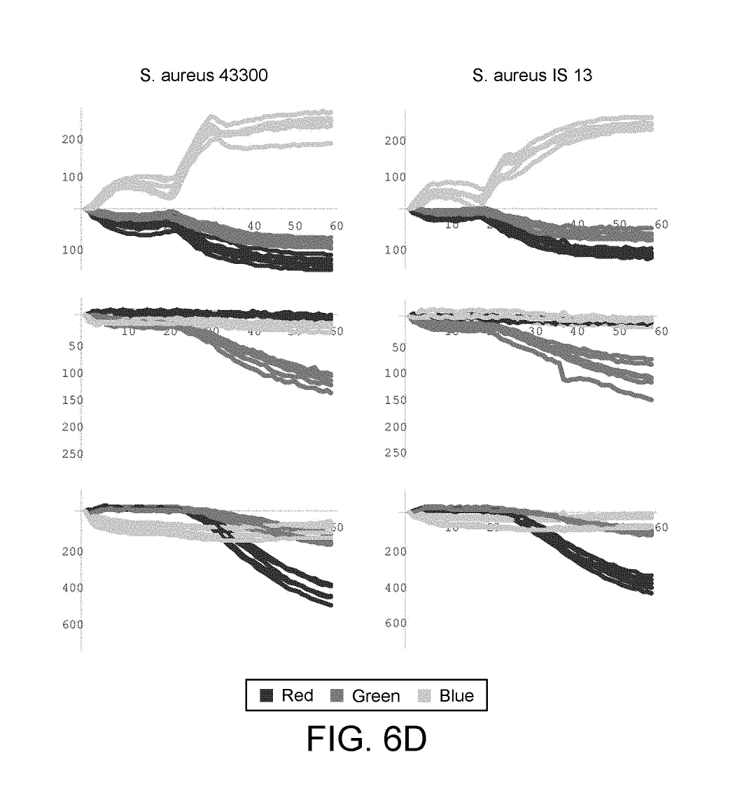

FIGS. 6A-D show strain-specific sensor patterns for S. aureus 25923, S. aureus 29213, S. aureus 43300, and S. aureus IS-13. Table 3 shows 100% accurate strain identification for 29 out of 31 strains of bacteria. (“IS-# refers to clinical isolate; other data represents ATCC reference strains.)

TABLE 3
Identification of Bacterial Strains.

| Species | Strain | Percent correct |
|---|---|---|
| A. xylosioxidans | IS-30 | 100 |
| A. xylosioxidans | IS-35 | 100 |
| A. xylosioxidans | IS-46 | 100 |
| A. xylosioxidans | IS-55 | 100 |
| P. aeruginosa | IS-15 | 100 |
| P. aeruginosa | IS-65 | 100 |
| P. aeruginosa | IS-22 | 100 |
| P. aeruginosa | IS-36 | 100 |
| S. aureus | 25923 | 100 |
| S. aureus | 29213 | 100 |
| S. aureus | 43300 | 100 |
| S. aureus | IS-13 | 100 |
| S. aureus | IS-38 | 100 |
| S. aureus | IS-43 | 100 |
| S. aureus | IS-70 | 100 |
| E. coli | 25922 | 100 |
| E. coli | 35218 | 100 |
| E. coli | 760728 | 94 |
| E. coli | IS-39 | 12.5 |
| E. coli | IS-44 | 100 |
| C. diversus | IS-01 | 100 |
| C. diversus | IS-28 | 100 |
| C. diversus | IS-31 | 100 |
| C. diversus | IS-33 | 100 |

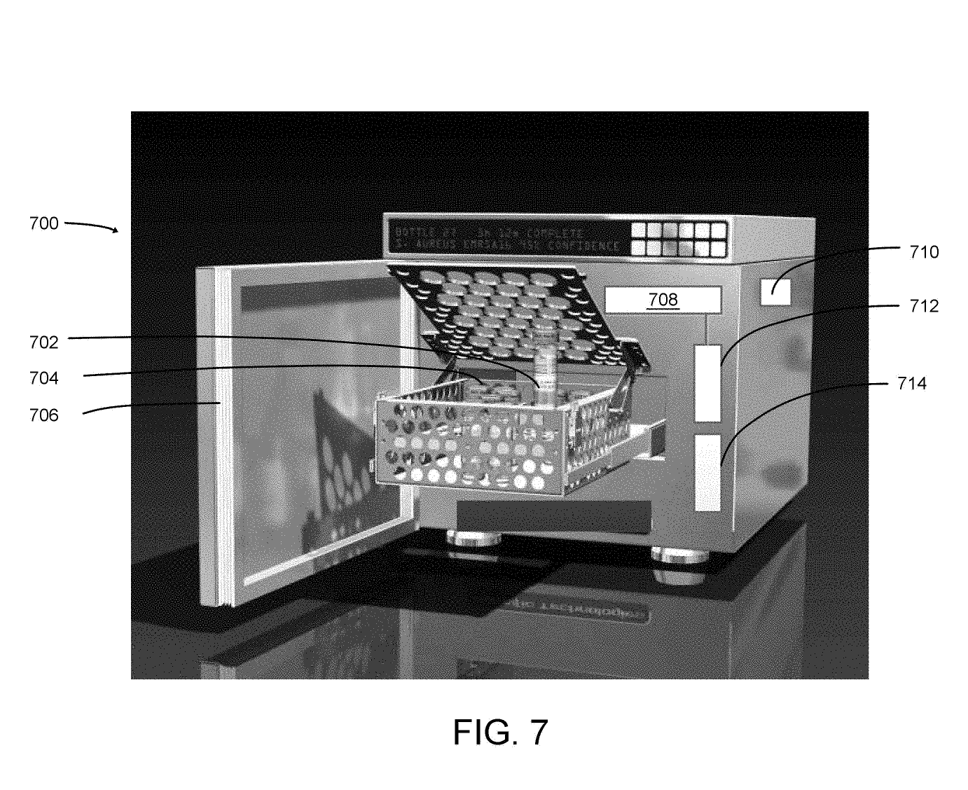

FIG. 7 depicts an example of an apparatus 700 for automated identification of microorganisms by species and/or strain and/or assessing a susceptibility or resistance of microorganisms. Containers 702 for culturing samples including microorganisms in the presence of colorimetric sensor array 704 are positioned in housing 706 of apparatus 700. Containers 702 may be of various designs configured to hold liquid or solid media, fluid or solid samples, or any combination thereof. Housing 706 also includes detector 708 operable to detect a change in one or more color components of each sensor of each sensor array 704. Detector 708 may be, for example, a scanner (e.g., a flatbed scanner). Apparatus 700 may also include thermostat 710 operatively coupled to a controlled-environment portion for incubating the samples.

Apparatus 700 may also include processor 712 configured to operate the detector 708 at selected time intervals, recording data to be manipulated by processor to generate temporal and/or static color response patterns. Apparatus 700 may also include memory storage device 714 operatively coupled to the processor that stores a multiplicity of temporal and/or static color response patterns of known species and/or strains of microorganisms (e.g., bacteria, yeast, protozoa). Thus, the system is operable to generate a temporal and/or static color response pattern of a sample including a microorganism, and automatically identify the microorganism (e.g., by species and strain) by comparing the generated color response pattern of the array 704 with the stored multiplicity of temporal and/or static color response patterns (e.g., the “library”) of known species and/or strains of microorganisms. Comparing the generated color response pattern with the library of known species and/or strains of microorganisms may be achieved by one of a number of statistical methods described herein or incorporated by reference.

In other embodiments, information output by detector 708 may be sent to a remote database to be processed and compared to a centralized database to determine the closest matching dataset. In other embodiments, certain portions of the calculation may be performed locally at a processor 712 on the apparatus 700 and some portions may be performed remotely by a processor 712 or other computing device on a server. In some embodiments, a library of datasets with previous data points for known antibiotic strains and/or known resistances or susceptibilities may be contained in apparatus 700 or in a centralized server. In the server embodiments, the data could be continually updated and stored as more assays are performed and organisms identified along with susceptibilities.

Apparatus 700 is also operable to assess susceptibility of the microorganism. In some embodiments, a second colorimetric sensor array will be utilized to assess susceptibility once the microorganism is identified. In some cases, the susceptibility/resistance assay follows species identification (e.g., in a blood culture without requiring growth of colonies in plate media), thus allowing rapid and cost effective determination of susceptibility and/or resistance. In certain cases, susceptibility is identified directly using the specimen obtained from a blood culture, allowing both species identification and susceptibility assay to be complete in less than 24 hours (including 10 hours for species/strain ID and a further 6-8 hours for susceptibility assay).

The memory storage device 714 may also store a multiplicity of temporal and/or static color response patterns of known microorganisms cultured in the presence of known antibiotics at known concentrations. In some embodiments, the data stored in memory device 714 may also include response patterns for known modes of antibiotic resistance at given concentrations. Thus, the system is operable to generate a temporal and/or static color response pattern of a sample including a microorganism, and automatically identify a susceptibility or resistance feature of the microorganism by comparing the generated color response pattern of the array 704 with the stored multiplicity of temporal and/or static color response patterns (e.g., the “library”) of known species and/or strains of microorganisms cultured in the presence of known antibiotics at known concentrations, and/or with known resistance or susceptibility modes. Comparing the generated color response pattern with the library of known modes of resistance or susceptibility may be achieved by one of a number of statistical methods described herein or incorporated by reference.

In some embodiments, a susceptibility assay and species and/or strain ID assay will be performed simultaneously. For instance, a susceptibility assay will include control samples that do not include an antibiotic substance. The colorimetric response of that colorimetric sensor array 704 in the control samples could be utilized to verify the ID of the microorganism. Simultaneously, the response of the colorimetric sensor array 704 to samples that are cultured with antibiotics or other substances could be utilized to determine a susceptibility or resistance of the microorganism to the applied antibiotics at the applied concentrations. In some embodiments, if susceptibility is determined prior to identification of the microorganism, a general mode of resistance may be identified based on the response of the colorimetric sensor array 704. The general mode of resistance may provide information regarding antibiotics likely to be more effective.

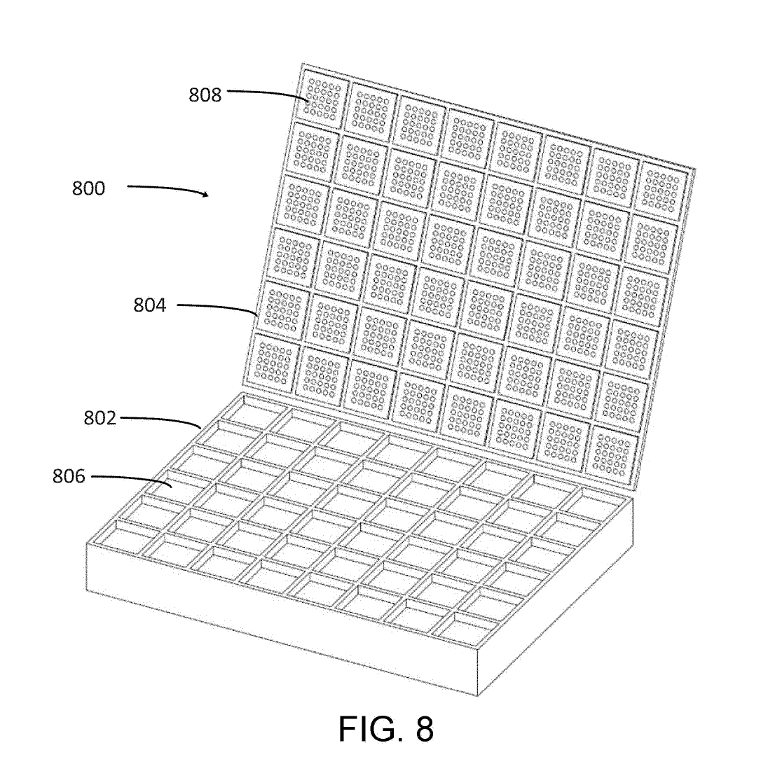

FIG. 8 depicts an embodiment of a container 800 configured for use to assess a susceptibility or resistance of microorganisms. Container 800 includes base 802 and lid 804, with wells 806 positioned in base 802 opposite colorimetric sensor arrays 808 on lid 804. Microorganisms may be placed in contact with growth medium (e.g., a solid or liquid growth medium) in wells 806. A substance (e.g., a drug such as an antibiotic) may be added to the growth medium. In one example, rows and columns of wells 802 in container 800 may be used for different microorganisms, different substances (e.g., drugs including antibiotics that may potentially kill the microorganisms), and/or different concentrations of substances. After samples are loaded on wells 802, a lid 804 may be positioned over base 802, such that each colorimetric sensor array 808 is proximate a well 806 and in gaseous (or liquid) communication. In other embodiments, various other configurations could be utilized to bring the gas and volatile organic compounds emitted from the sample into gaseous proximity of the sensor array 808 at sufficient gaseous concentrations. The response of the sensor arrays 808 recorded by various detectors may then be assessed in order to determine a susceptibility or resistance of the various antibiotics or substances cultured with the sample. Susceptibility may be assessed via temporal response of the sensors in colorimetric sensor arrays 808 as described herein. Container 800 can be positioned in an apparatus (e.g., apparatus 700) for automated assessment of susceptibility and/or resistance.

In some embodiments, susceptibility and/or resistance of a sample may be assessed by preparing a matrix of wells including a mixture of growth media and an antibiotic at different concentrations in each well and including various controls. The sample may be prepared direct from a human specimen, for example, a tissue (e.g. blood) from a human may be directly deposited into a culture medium in a well in the matrix. In other embodiments, a sample from a human or other mammal may first be cultured to grow any microorganisms in the sample to a level sufficient for susceptibility testing. Then, a portion of the culture medium containing the microorganisms would be removed from the culture, and deposited into a culture medium in a well including either a substance such as an antibiotic or no substance (i.e. control). The colorimetric response may be utilized to determine a gradient of responses of the antibiotics at various concentrations. Then, from the various responses at various concentrations, an optimal antibiotic and dosage amount may be selected for treating a patient from which a sample was extracted. In other embodiments, known microorganisms may be screened in this manner to determine substances to which the microorganisms are susceptible.

### MIC

In some embodiments, a minimum inhibitory concentration or (“MIC”) may be determined using the matrix of wells 806 and colorimetric sensor arrays 808. For example, a multiplicity or matrix of wells 806 may include various antibiotics or a certain subset of antibiotics at various concentrations. The concentrations may be selected at regular intervals or dosages to determine a minimum inhibitory concentration given the likely ranges of MICs for certain antibiotics and infections. A technician or automated process may incorporate a sample into the different wells 806. Then, the colorimetric response of the arrays 808 may be monitored as described herein at regular intervals. Additionally, controls without antibiotics may be included in separate cells to provide a basis for comparison, particularly for the samples that show partial susceptibility or resistance. The colorimetric response recorded for the colorimetric sensor array 808 for each well 806 at each concentration may be recorded at regular intervals which may be 10 minutes, 20 minutes, 30 minutes, 1 hour or other suitable time frame.

Generally, the wells 806 that do not produce a colorimetric response from the colorimetric sensor array 808 will generally indicate that the microorganism included in the sample is susceptible to that antibiotic at that concentration. In some embodiments, the colorimetric sensor array 808 may exhibit a partial response to the antibiotic indicating a partial resistance or susceptibility to that antibiotic at that concentration. Accordingly, based on the response the potential infection may be characterized as (1) fully susceptible, (2) partially susceptible, or (3) resistant to the antibiotic at that range. This information may be important to extrapolate between concentrations to determine an optimal antibiotic, or only use antibiotics at concentrations at which the organism is fully susceptible.

Based on the information from the colorimetric sensor array, minimum inhibitory concentrations for one or several antibiotics may be determined. With that information, a caregiver could administer an effective dosage regimen or treatment to a person or mammal from which the sample came in order to treat the potential infection. In other embodiments, the information may be extrapolated or interpreted to determine the optimal antibiotic and/or concentration.

### Susceptibility Signature

In some embodiments, the response of the colorimetric sensor array 808 may provide additional information beyond just that the infection or microorganism has grown and is emitting volatile organic compounds that are detected by the colorimetric sensor arrays 808. This may include a susceptibility signature, or additional information in the response of the sensor array 808 to the emitted volatile organic compounds in samples that have antibiotics applied. This information may be utilized to determine more granular data about the susceptibility of the microorganism to the antibiotic, or perhaps the resistance.

For instance, the response of the colorimetric sensor array 808 may indicate the mode of resistance exhibited by the microorganism. For example, there are several known modes through which microorganisms may be resistant to antibiotics. These include (1) production of enzymes that deactivate the antibiotics, (2) alteration of the binding site of the antibiotic, (3) alteration of a metabolic pathway with which the antibiotic interferes, (4) development of cell envelop layers that are impermeable to the antibiotic (or several), and (5) pumps (e.g., efflux) that pump out harmful substances in the microorganism which may confer multi-drug resistance. The response signature of the colorimetric sensor array 808 to a given antibiotic at a given concentration may provide information about potential resistance modes of resistance of the microorganism to the antibiotic. Additionally, the aggregate response in the presence of different antibiotics at different concentrations may also provide information about the modes of resistance.

Information about the modes of resistance or determining a mode of resistance may be advantageous to selecting an appropriate or effective antibiotic or antibiotic cocktail to treat a patient. For instance, the mode of resistance may be relevant to determining or extrapolating the response of the colorimetric sensor array 808 to determine the optimal antibiotic and/or concentration of that antibiotic. In some embodiments, this could be determined prior to or in parallel with identifying the microorganism. For instance, if the response of the colorimetric sensor array 808 indicates that the mode of resistance of the antibiotic is an efflux pump, or similar multi-drug resistant pump, an appropriate increase in concentration and/or cocktail of antibiotics could be administered. The increase in concentration of an antibiotic if it is determined that an efflux pump is present may be greater than for other modes of resistance.

In other examples, if the response to the colorimetric sensor array 808 indicates that the mode of resistance is a membrane or layer of the cell envelope that has developed, an antibiotic may be selected that is permeable to the new layer. In some embodiments, a library may include information about the mode of resistance and potential antibiotics or antibiotic classes that may be more effective against certain resistance modes.

In some embodiments, a library of known or determined susceptibilities may be developed that are associated with the signature of the response of the colorimetric sensor array 808 after contact with the volatile organic compounds emitted from certain microorganisms. This may include microorganisms incubated either in absence or in the presence of antibiotic substances. Accordingly, a phenotype of resistances and/or susceptibilities may be determined based on the signature of the microorganisms that is independent of the identification of the microorganism. This database could be regularly updated as various new susceptibilities and resistances are detected, and/or could be comprised of data assembled by sequential laboratory testing.

Accordingly, utilizing the susceptibility or resistance dataset, a signature of colorimetric sensor array 808 response from a sample suspected of containing a microorganism may be compared to the dataset. This comparison may be able to identify a susceptibility to a substance or a list of potential susceptibilities to various substances of a microorganism in the sample.

### Susceptibility Score

The response to the colorimetric sensor array 808 may also be quantified as a numeric value into a susceptibility score for each applied antibiotic at each concentration. The susceptibility score could be a weighted average of various factors that could include: (1) the average sensor 808 response, (2) the temporal response of the sensor 808, for instance the rate of growth (3) the signature of growth, (4) the amount of time it took for the sensor 808 response to be determined, (5) the concentration of antibiotic utilized, (6) the starting concentration of the microorganism, and other factors. Additionally, a susceptibility score may be modified based on other information determined from the colorimetric sensor 808 response, including the mode of any potential resistance. The susceptibility score could be displayed as a numerical value, a color map, a heat map of potential antibiotics, or another display mechanism.

In other embodiments, the score or indication provided from the susceptibility testing may include whether there is (1) complete resistance, (2) partial resistance, or (3) complete susceptibility. In some embodiments, there may be various thresholds of sensor 808 response that triggers the outcome to be one of the three categories. In some embodiments, responses to certain indicators may have more or less weighting in determining susceptibility to antibiotics. In other embodiments, the susceptibility testing may return a list of antibiotics with minimum inhibitory concentrations. In some embodiments, the list of antibiotics may be ranked according to various factors including kill time, absolute level of response over time.

In some embodiments, both turbidity and colorimetric sensor 808 response information could be combined to achieve greater granularity on the susceptibility and/or resistance. In some embodiments, a system may be provided that measures both the colorimetric response and the turbidity using the same optical detector 708. In other embodiments, two optical detectors 708 may be utilized to determine both the turbidity and the colorimetric sensor 808 response.

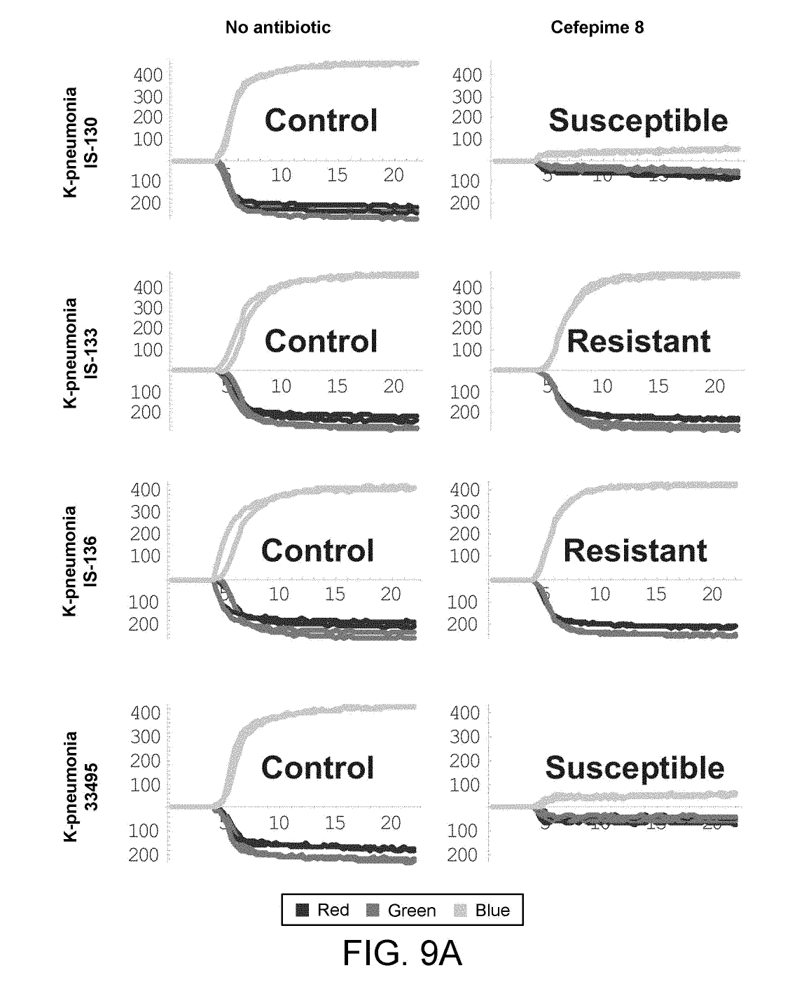

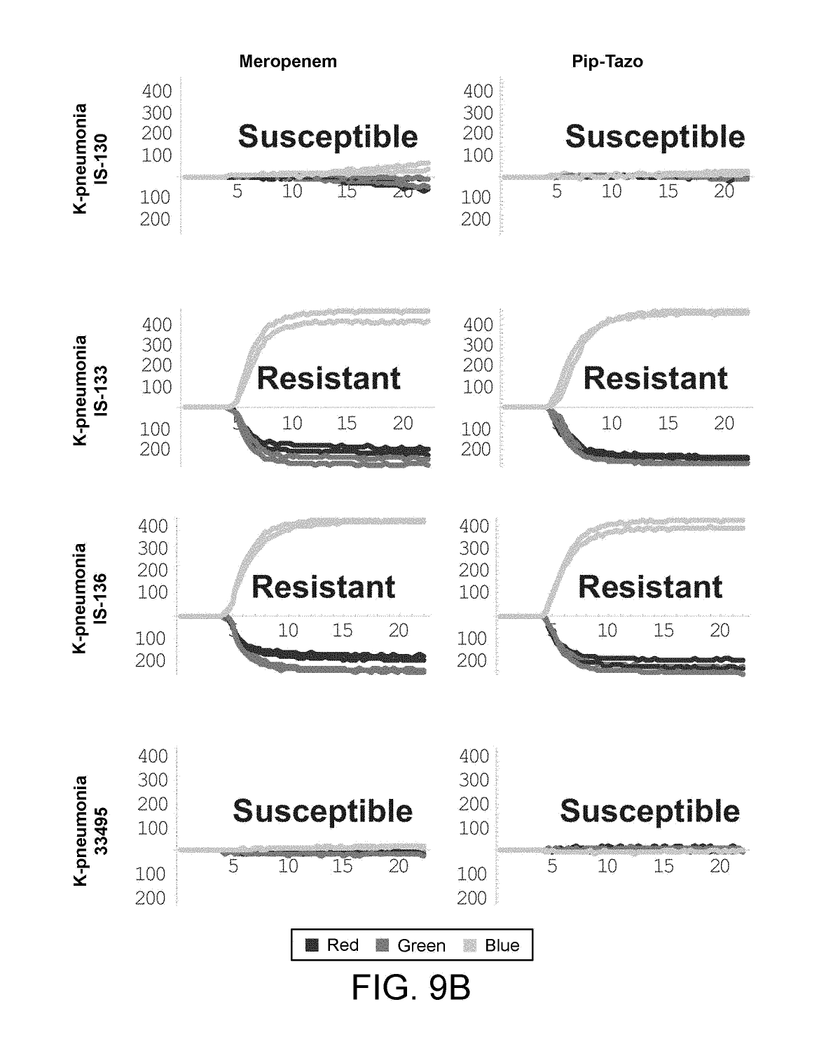

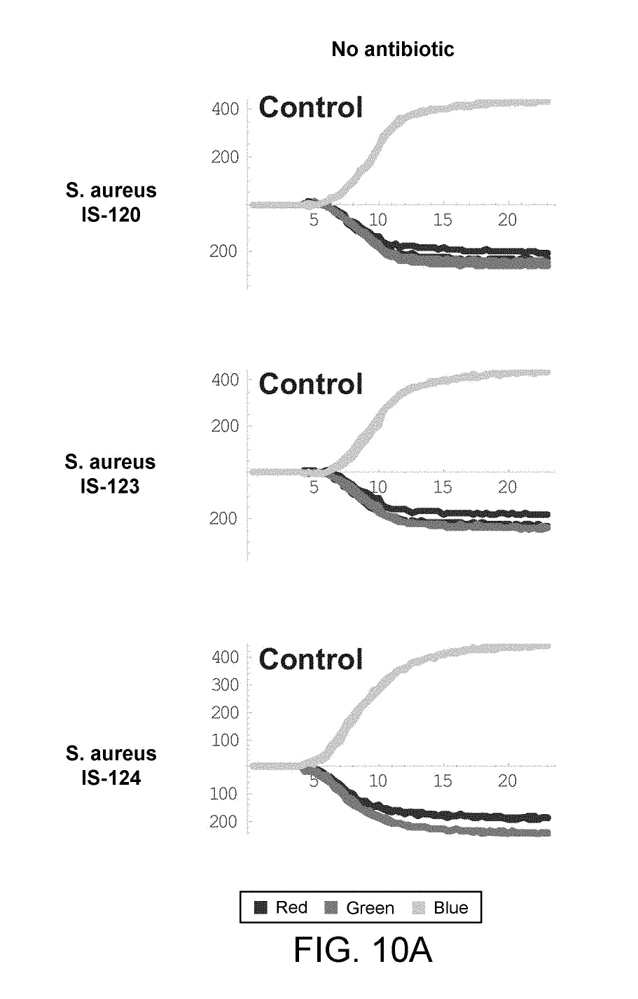

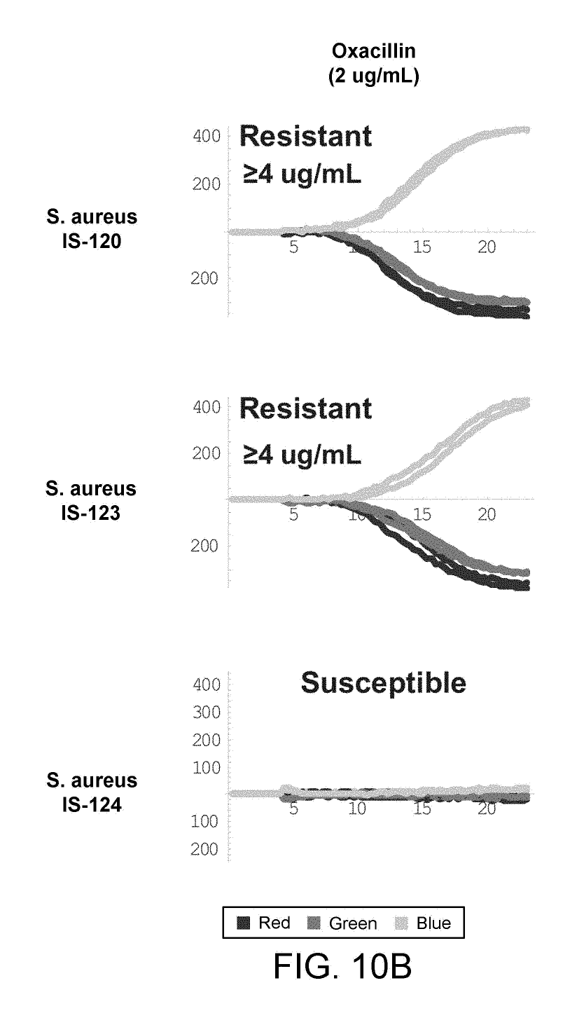

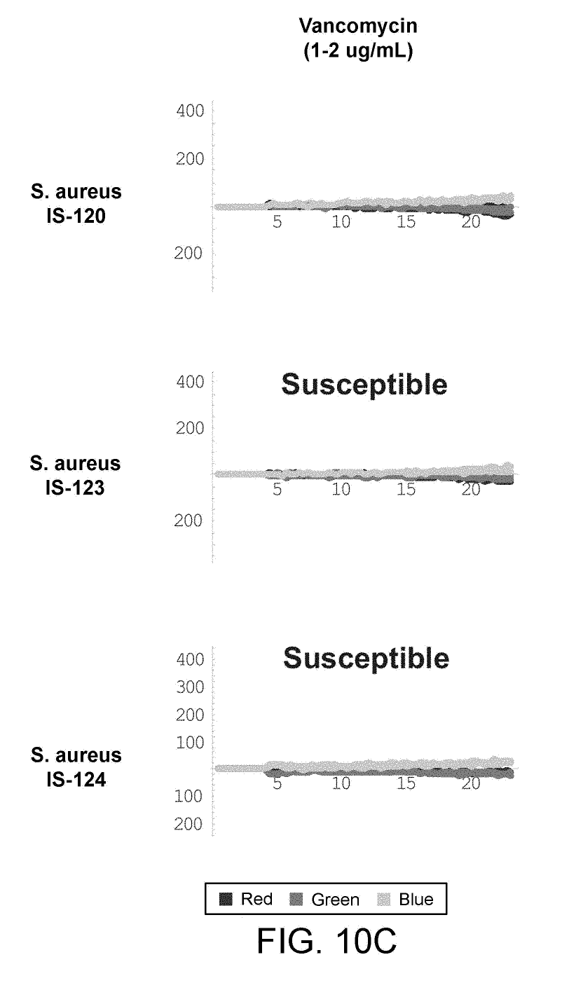

FIGS. 9A-B show susceptibility of K. pneumoniae strains obtained in 6 hours in Muller-Hinton agar (x axis indicates time in hours). Susceptibility is indicated by a lack of metabolic products compared to controls (no antibiotic). Antibiotic resistance is indicated by the presence of metabolic products on a scale comparable to that of the control. FIGS. 10A-C show susceptibility of S. aureus strains obtained in 6 hours in Muller-Hinton agar.

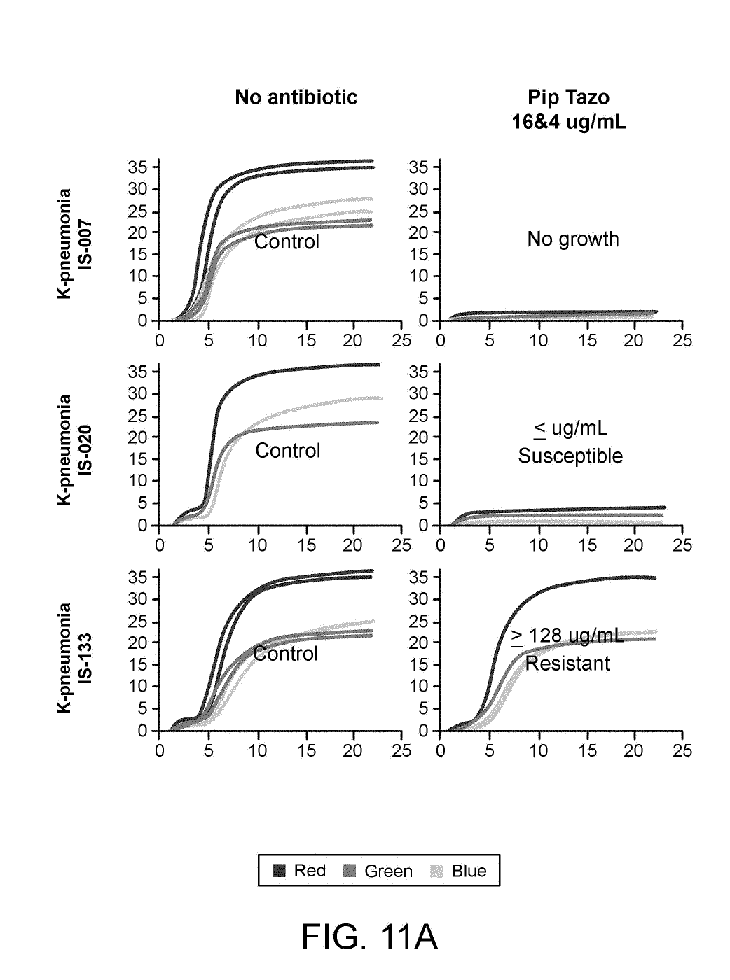

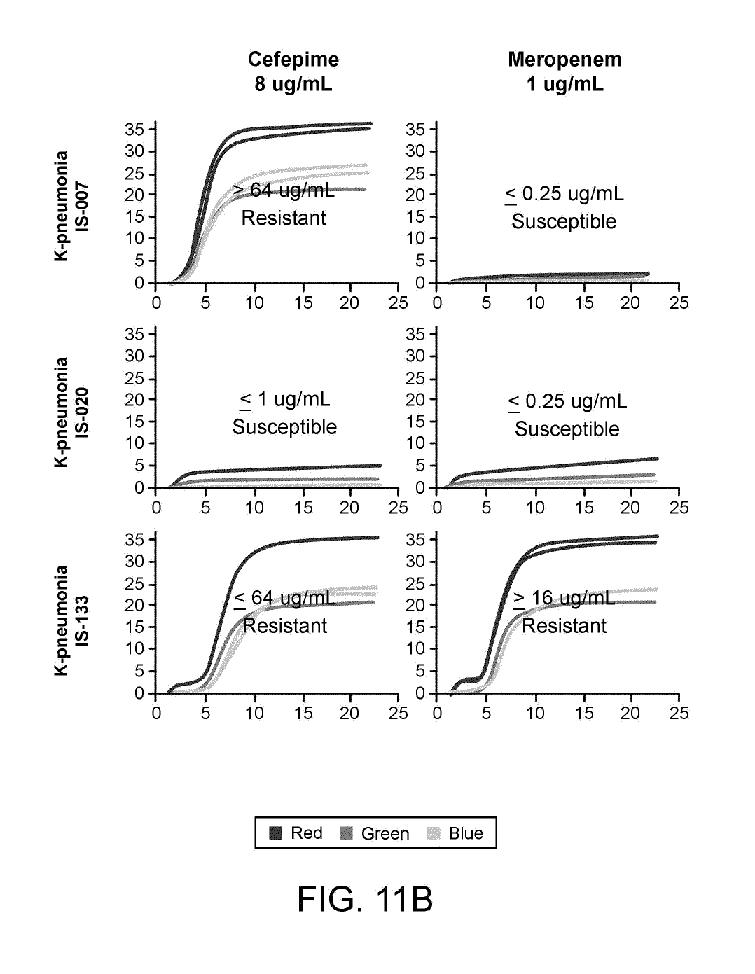

In another susceptibility test, three strains of K. pneumoniae were inoculated onto the solid growth media with no antibiotic (control), piperacillin/tazobactam (PIP/TAZO) at concentrations of 16 ug/ml and 4 ug/ml, Cefepime at 8 ug/ml and Meropenem at 1 ug/ml. Temporal results are shown in FIGS. 11A-B. For K. pneumoniae IS-007, results show susceptibility to PIP/Tazo (at 16 ug/ml and 4 ug/ml) and Meropenem (at 1 ug/ml) and resistance to Cefepime at 8 ug/ml. These results are in agreement with known susceptibility information that this strain is resistant to Cefepime below 64 ug/ml & susceptible to PIP/Tazo at 4 ug/ml, Cefepime <0.25 ug/ml. K. pneumoniae IS-020 was susceptible to all 3 antibiotics in agreement with known susceptibility information that this strain is susceptible to PIP/Tazo at <4 ug/ml, Cefepime <1 ug/ml and Meropenem at <0.25 ug/ml). K. pneumoniae IS-133 is known to be resistant at concentrations of PIP/Tazo below 128 ug/ml, Cefepime below 64 ug/ml and Meropenem below 16 ug/ml.

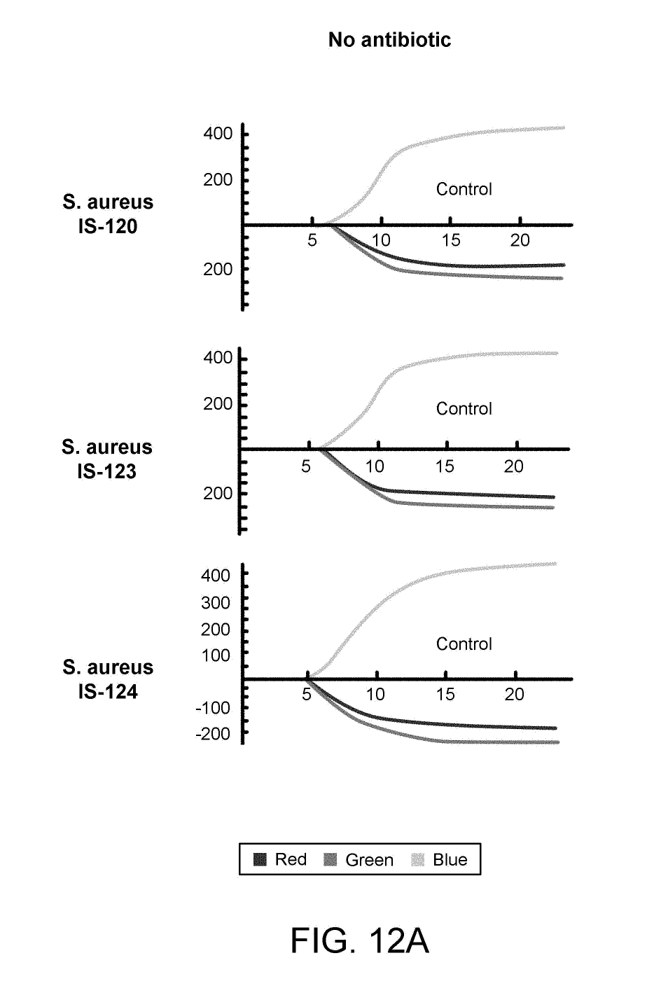

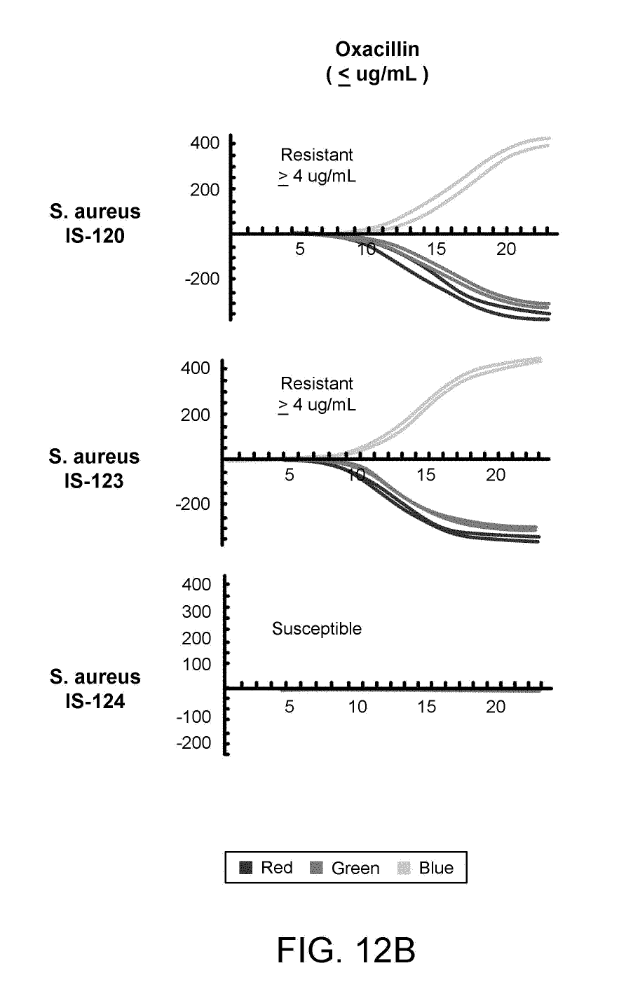

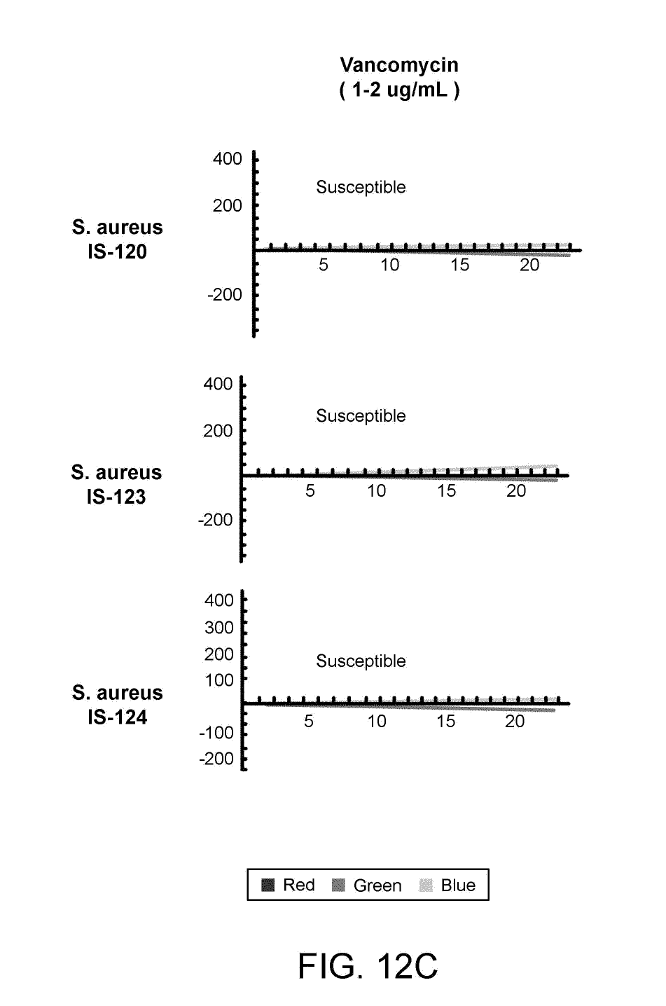

In another susceptibility test, three strains of S. aureus were inoculated onto the solid growth media. Temporal results are shown in FIGS. 12A-C. S. aureus IS-120 shows resistance to Oxacillin (at 2 ug/ml) and susceptibility to Vancomycin (at 1-2 ug/ml). These results are in agreement with known susceptibility information that this strain is resistant to Oxacillin below 4 ug/ml and susceptible to Vancomycin. S. aureus IS-123 shows resistance to Oxacillin (at 2 ug/ml) and susceptibility to Vancomycin (at 1-2 ug/ml). These results are in agreement with known susceptibility information that this strain is resistant to Oxacillin below 4 ug/ml and susceptible to Vancomycin. S. aureus IS-124 was susceptible to both antibiotics in agreement with known susceptibility information that this strain is susceptible to Oxacillin and Vancomycin.

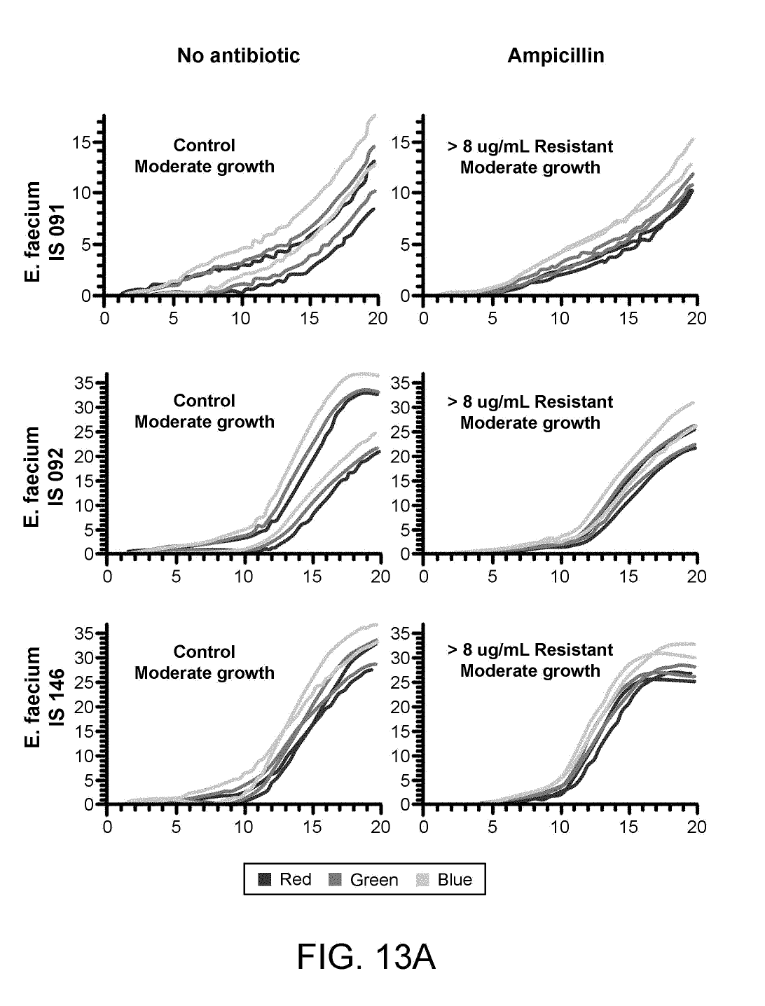

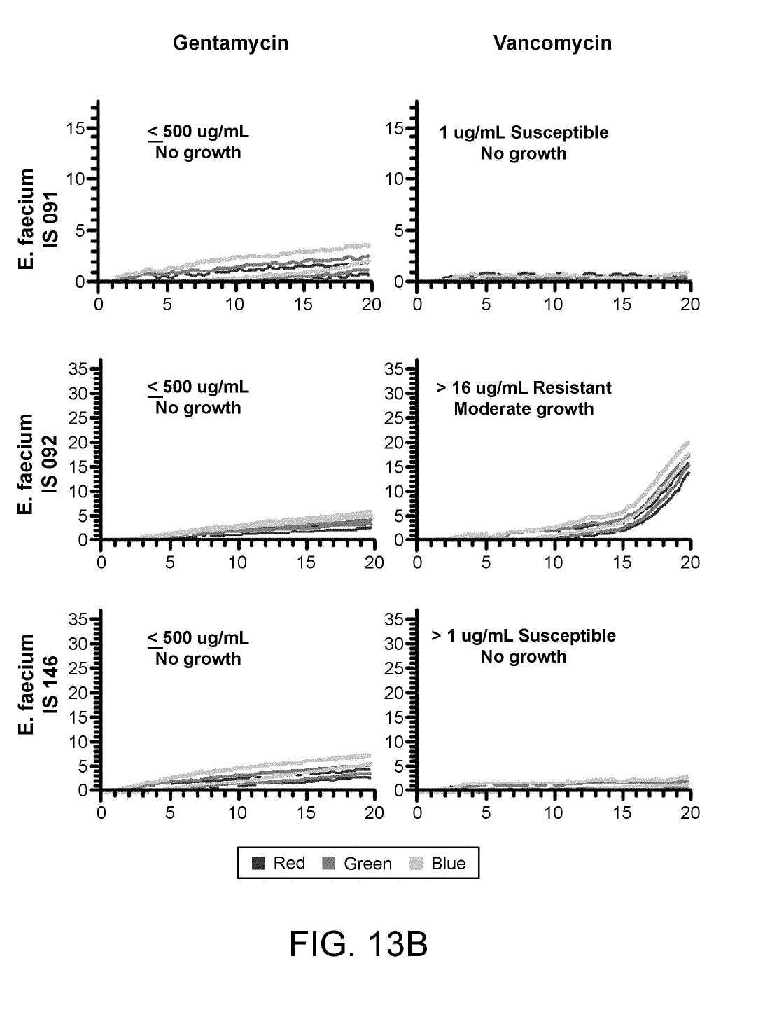

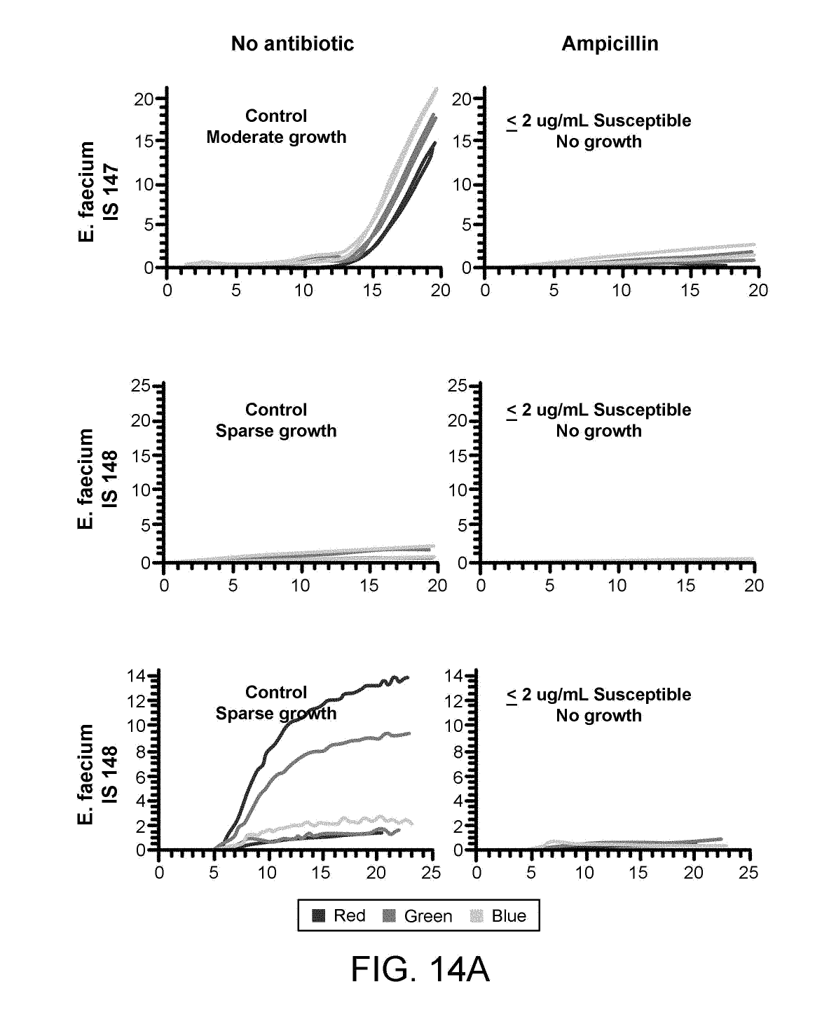

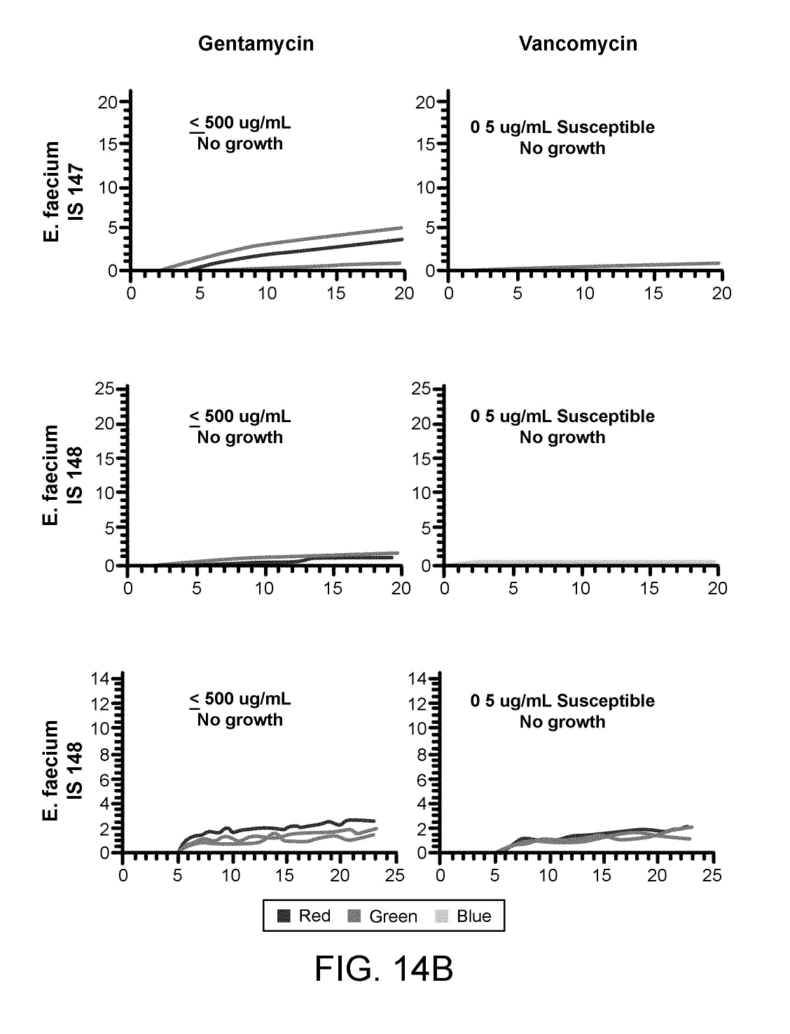

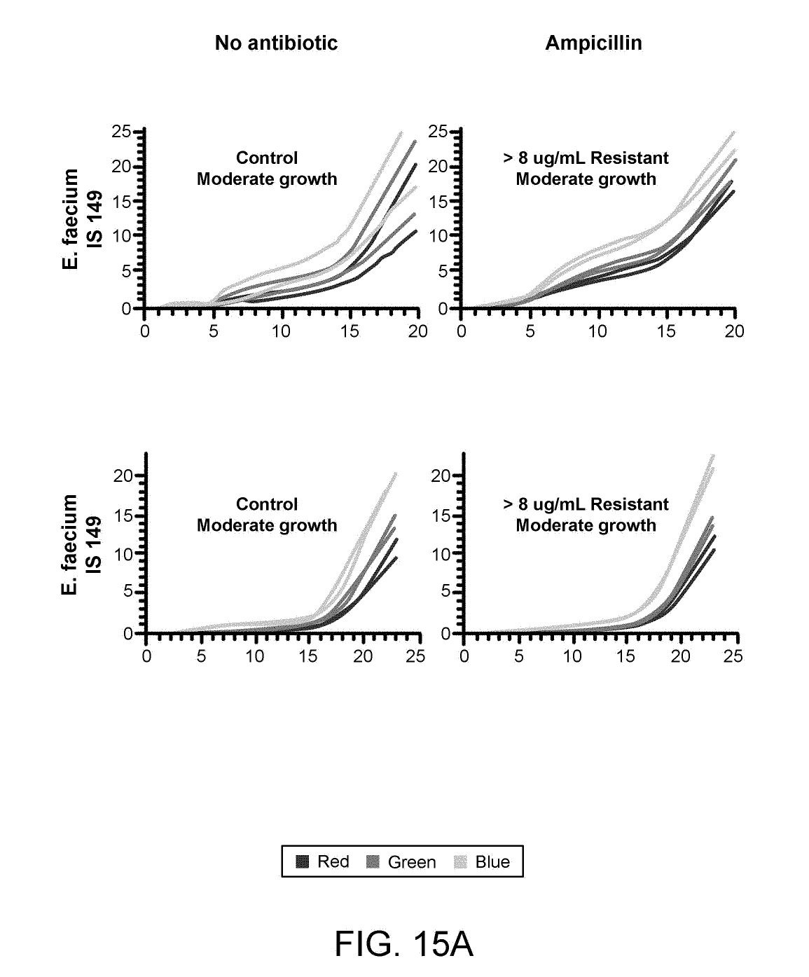

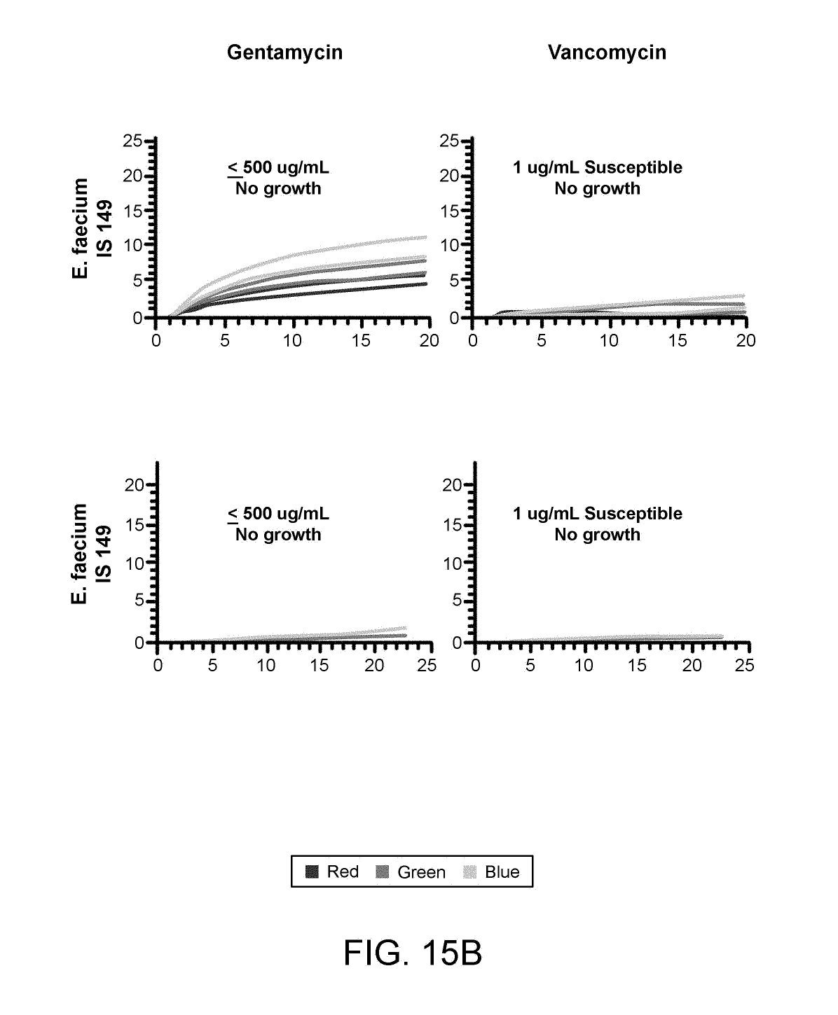

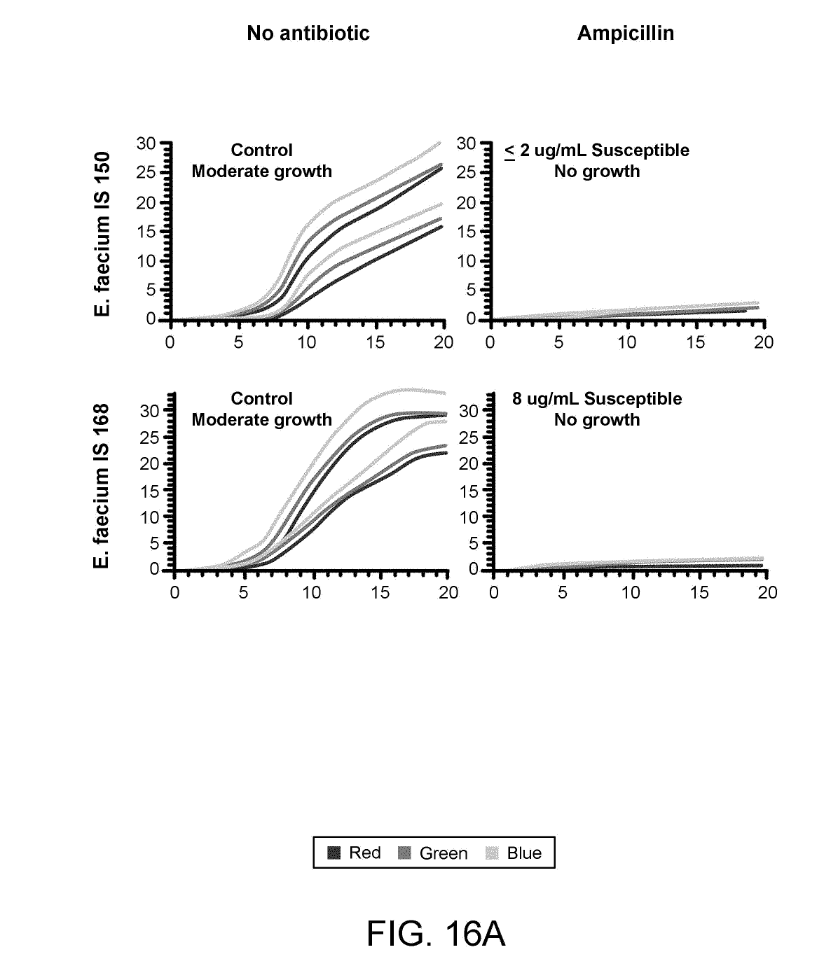

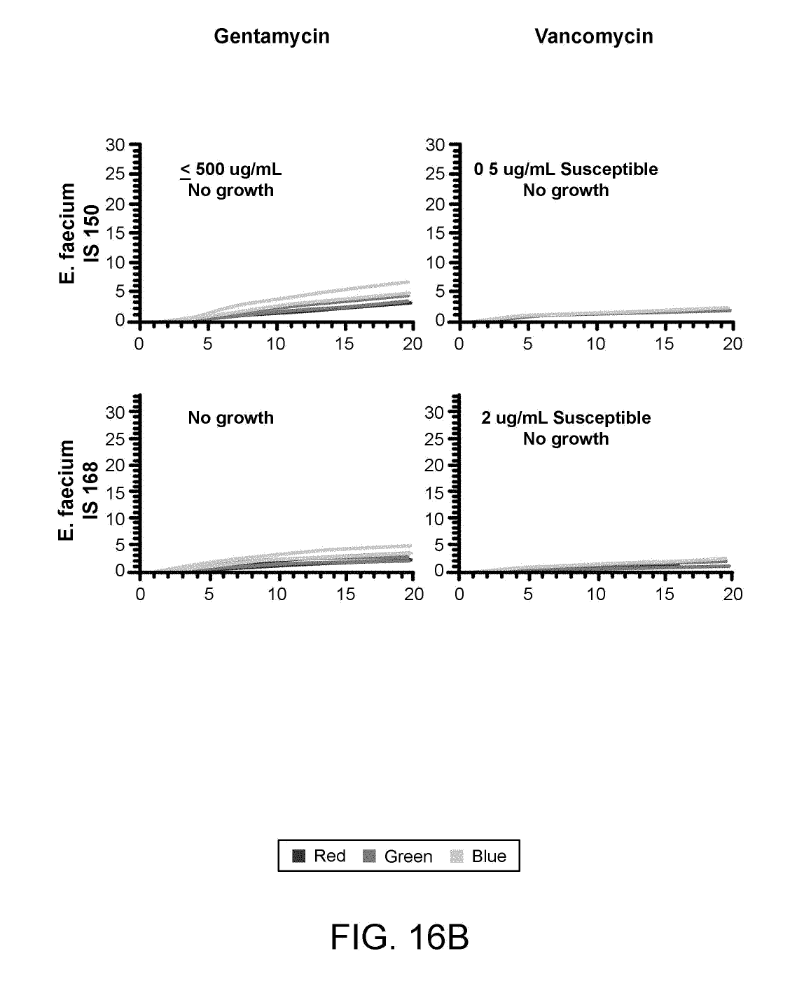

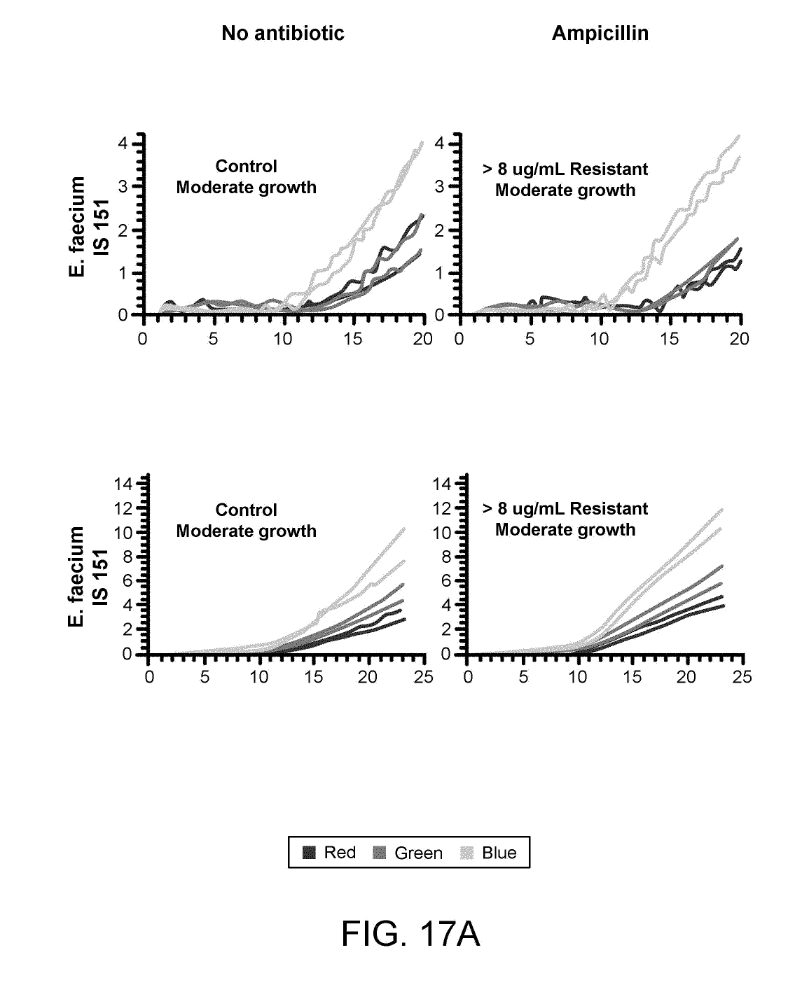

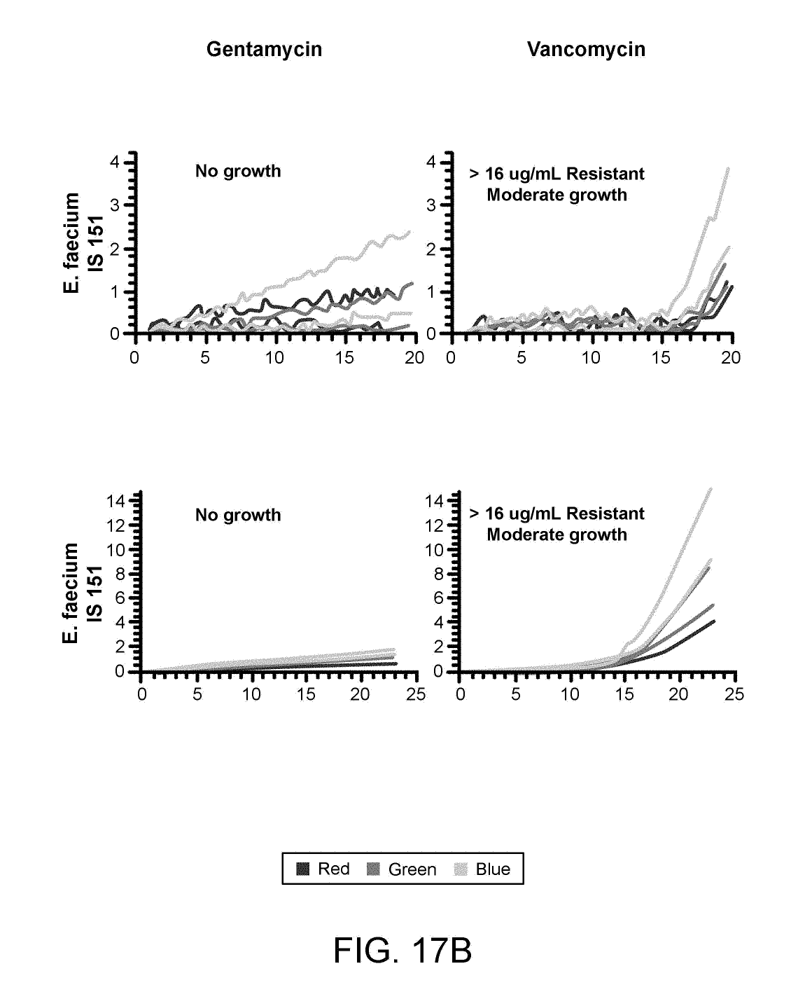

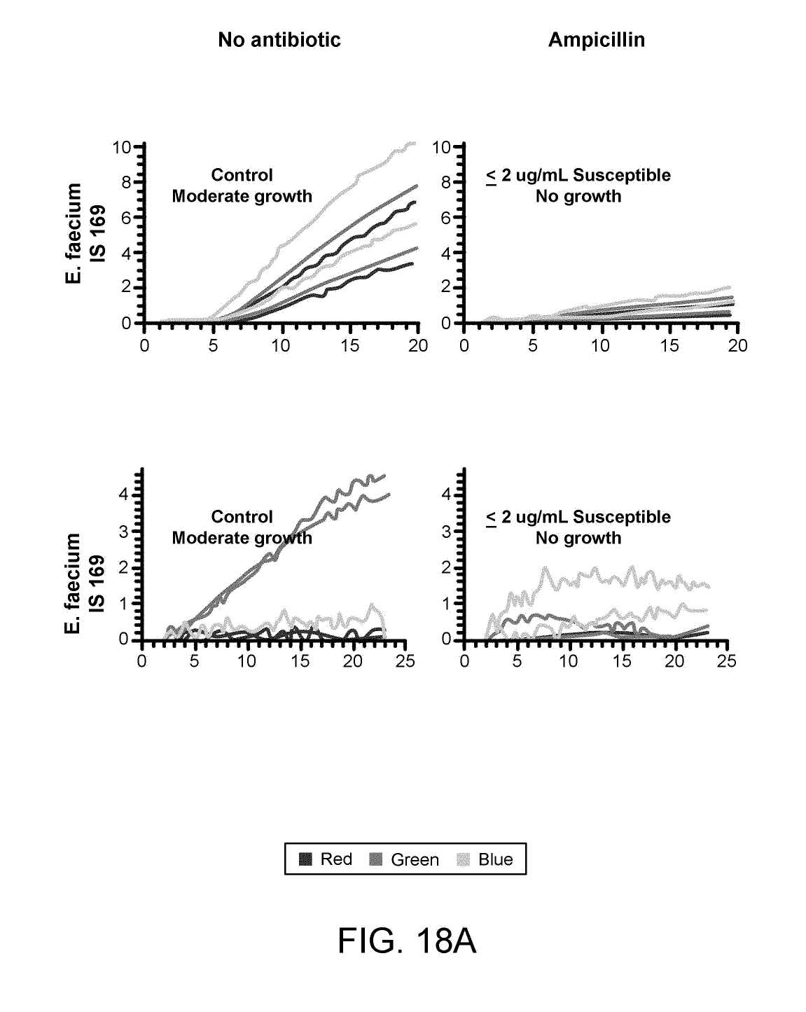

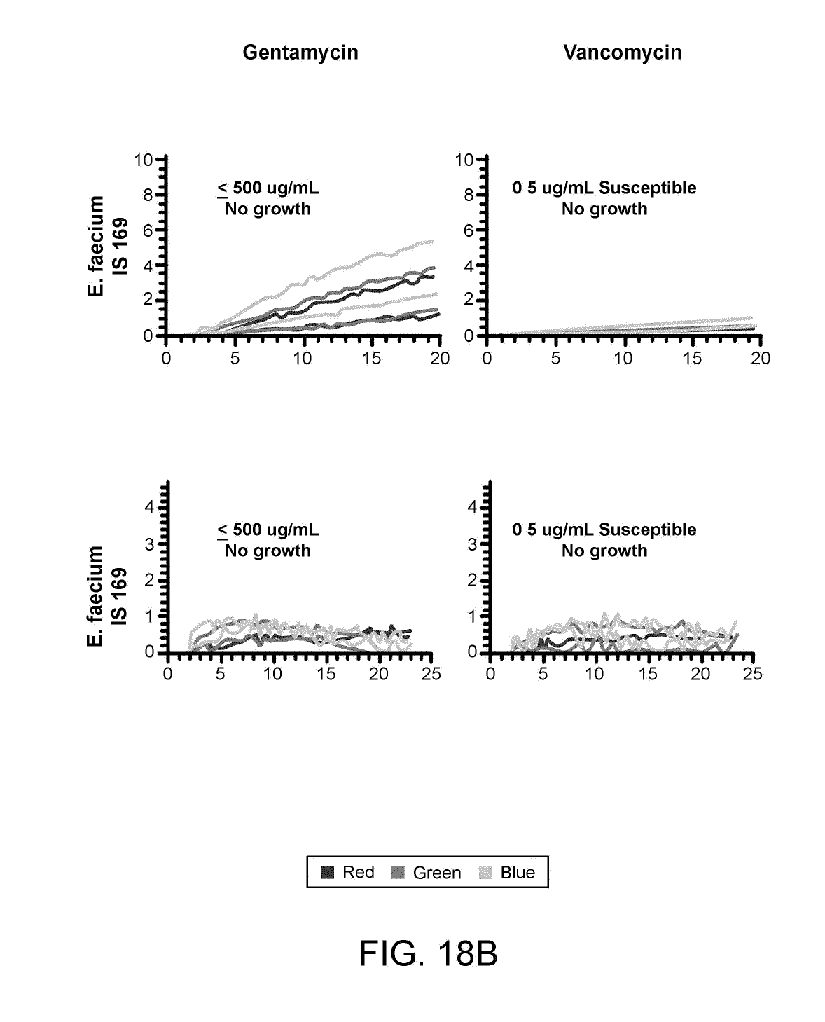

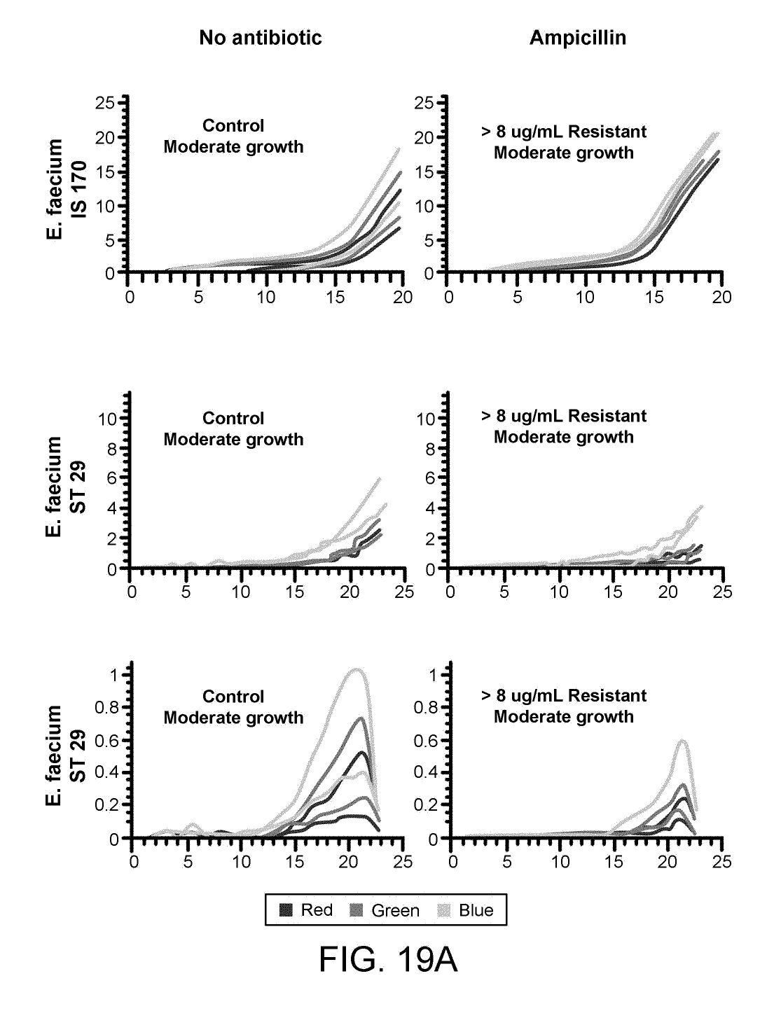

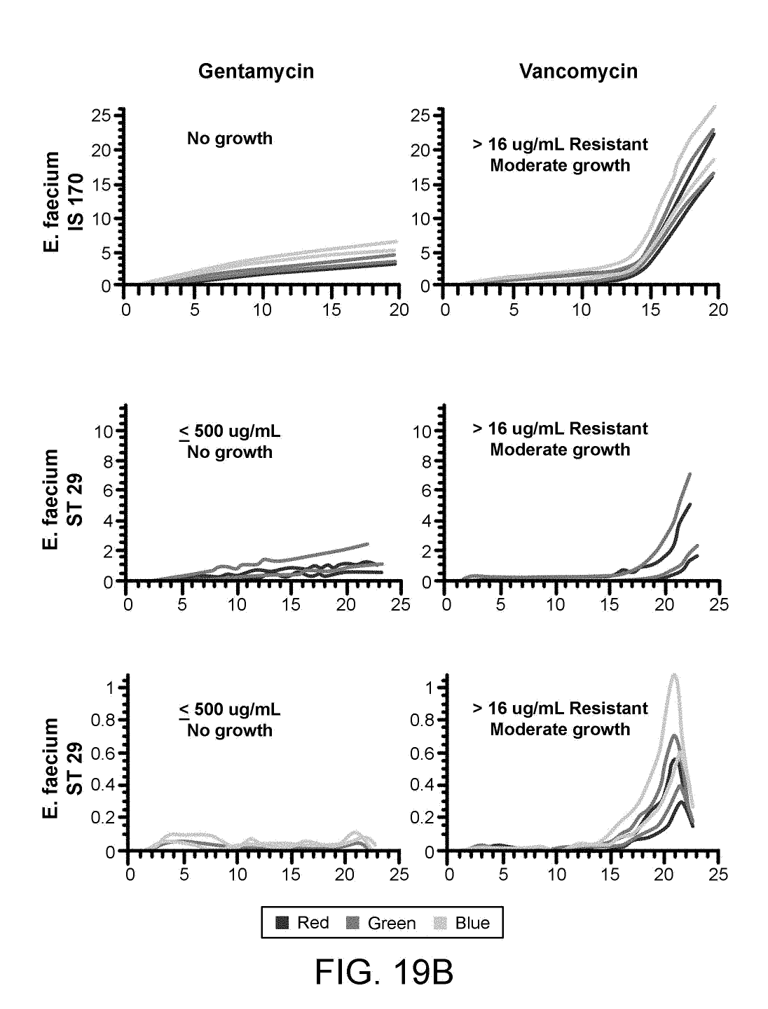

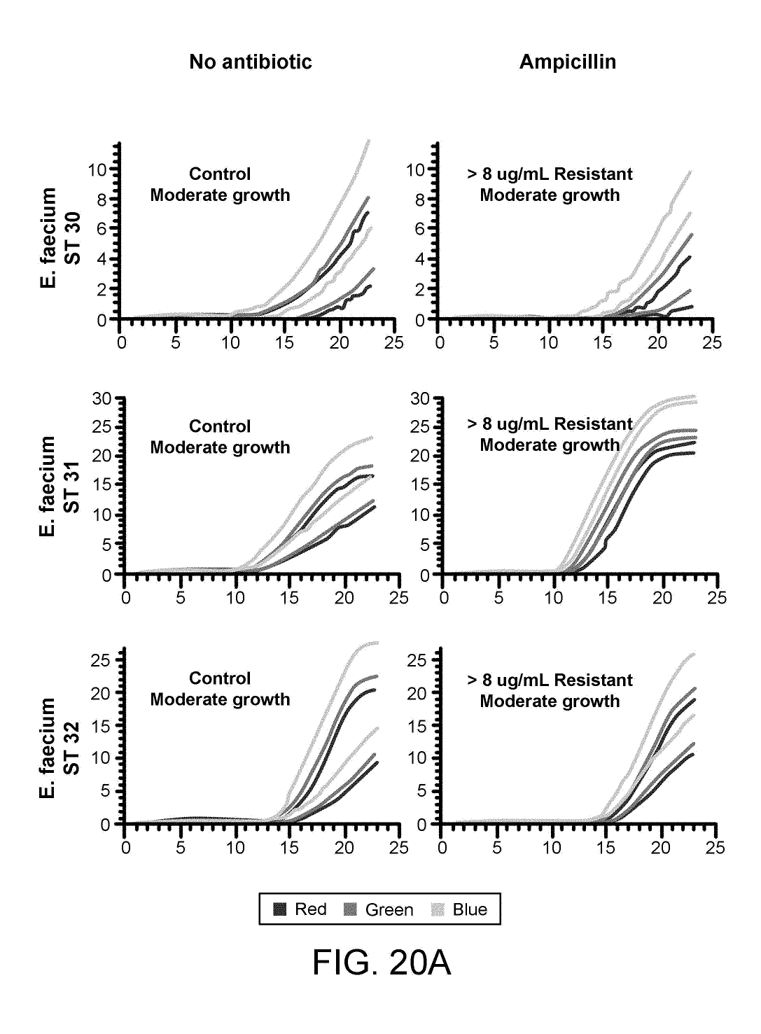

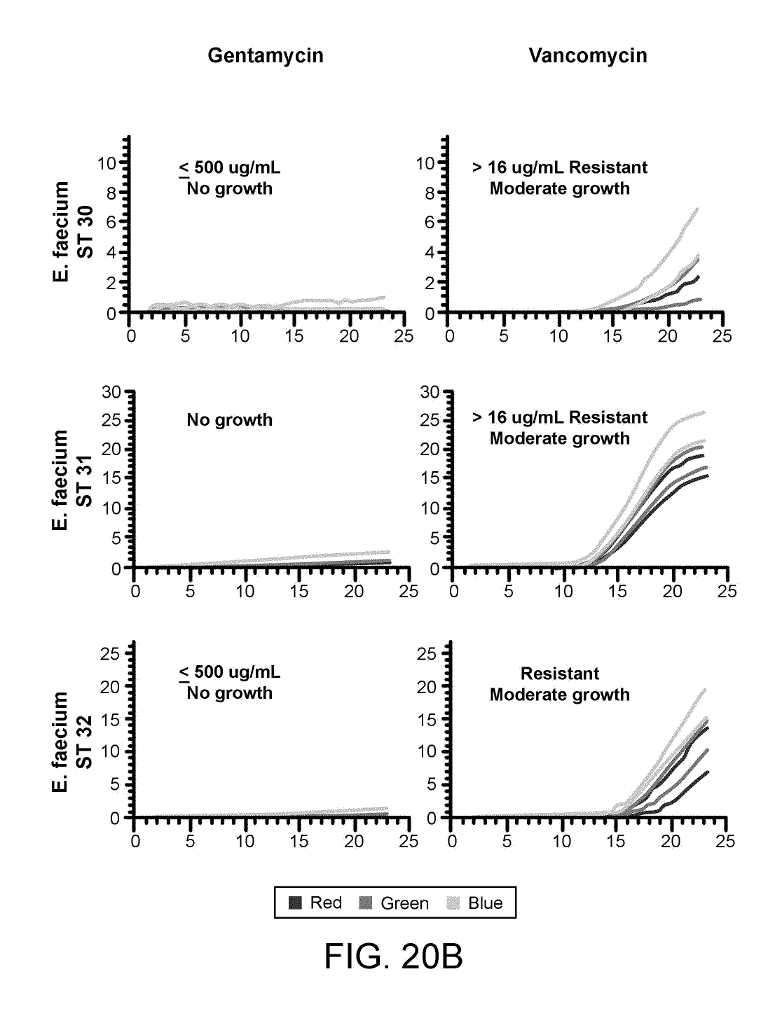

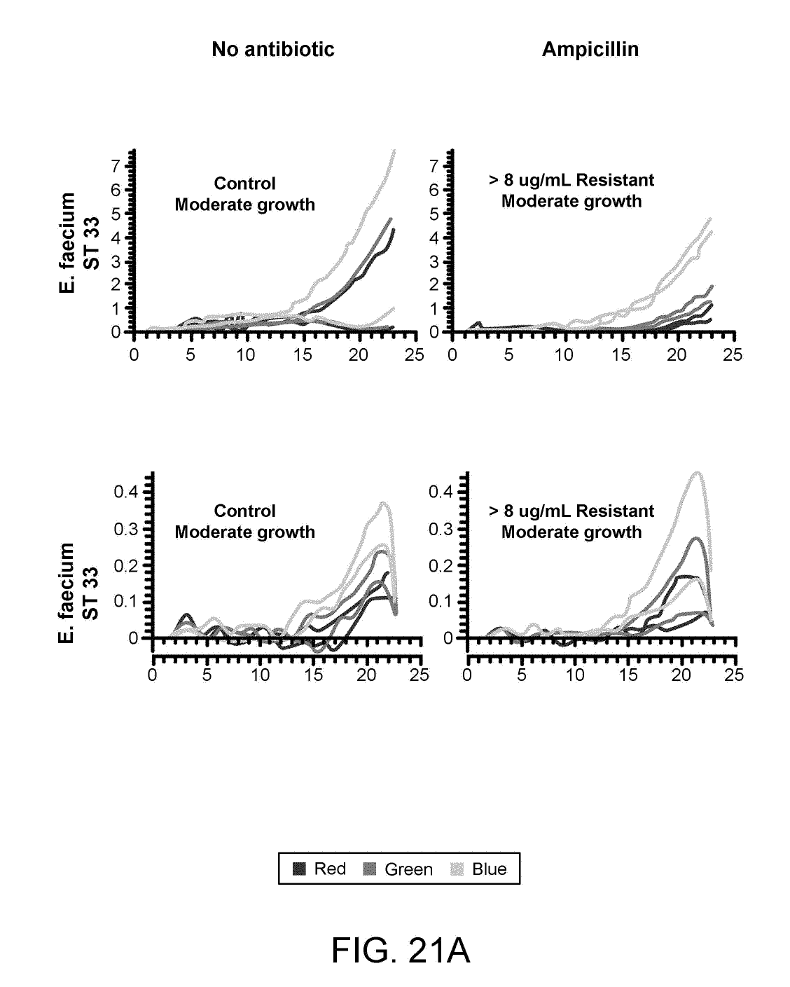

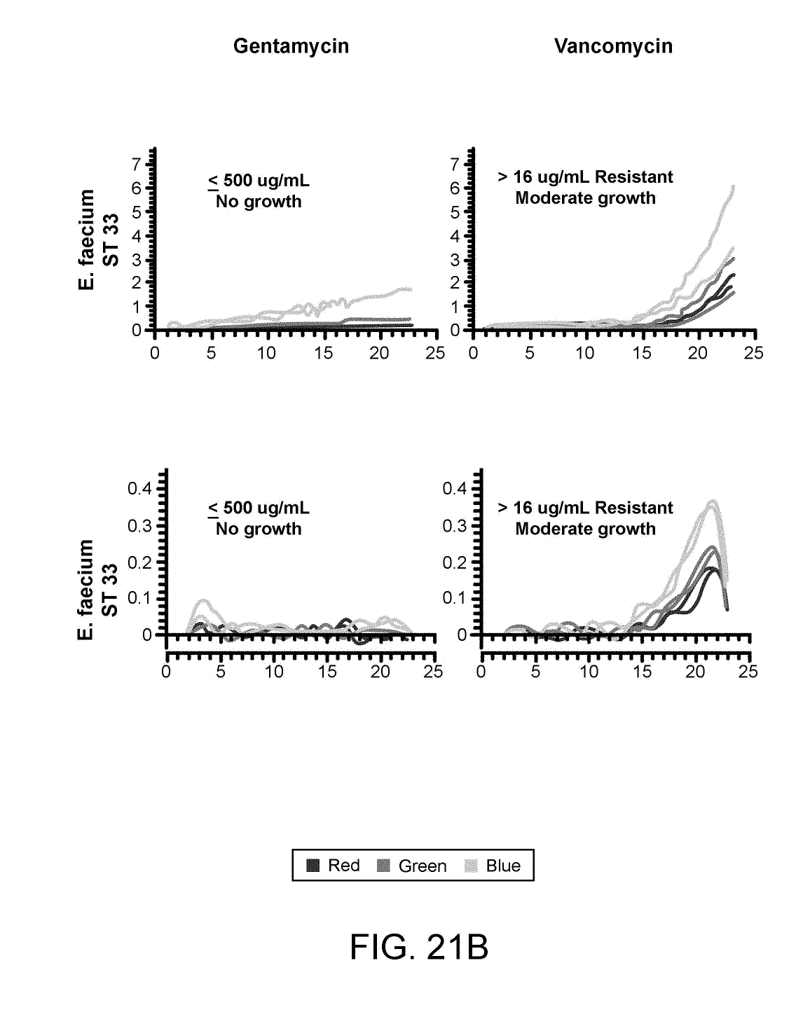

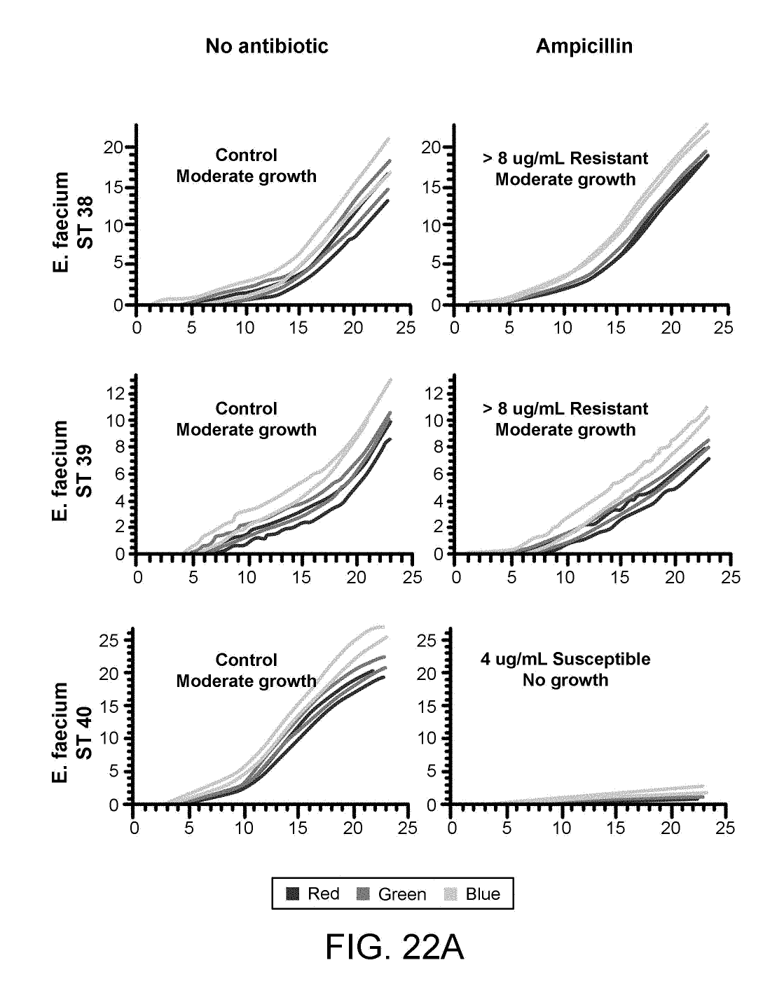

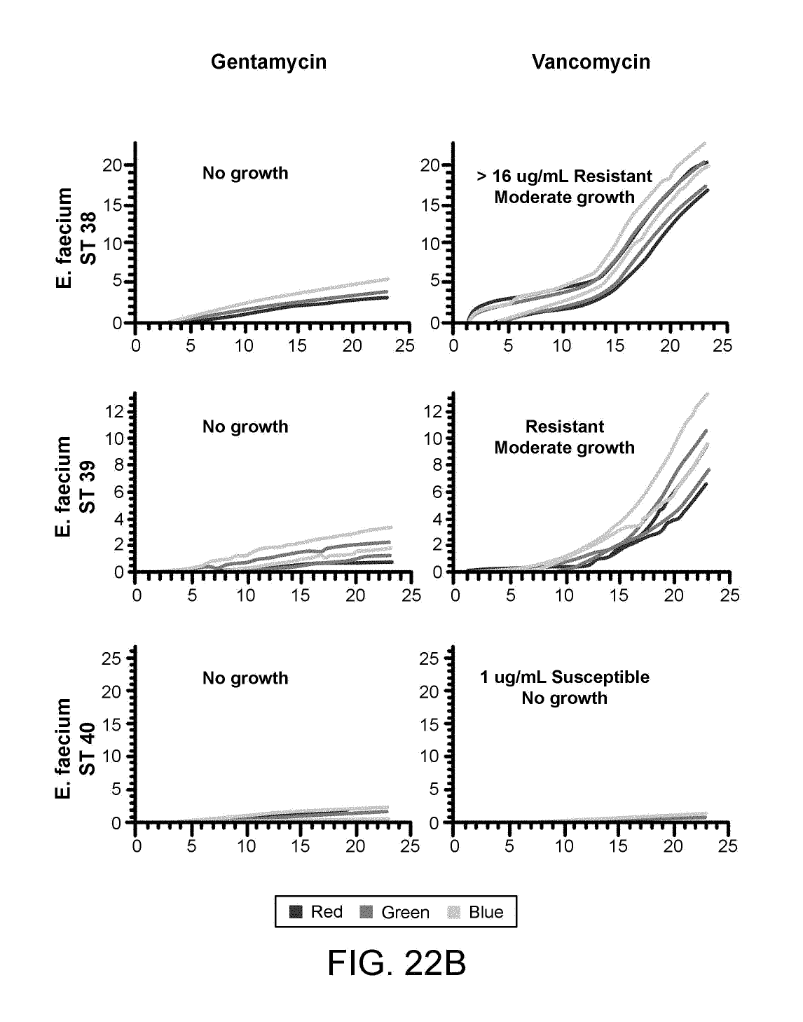

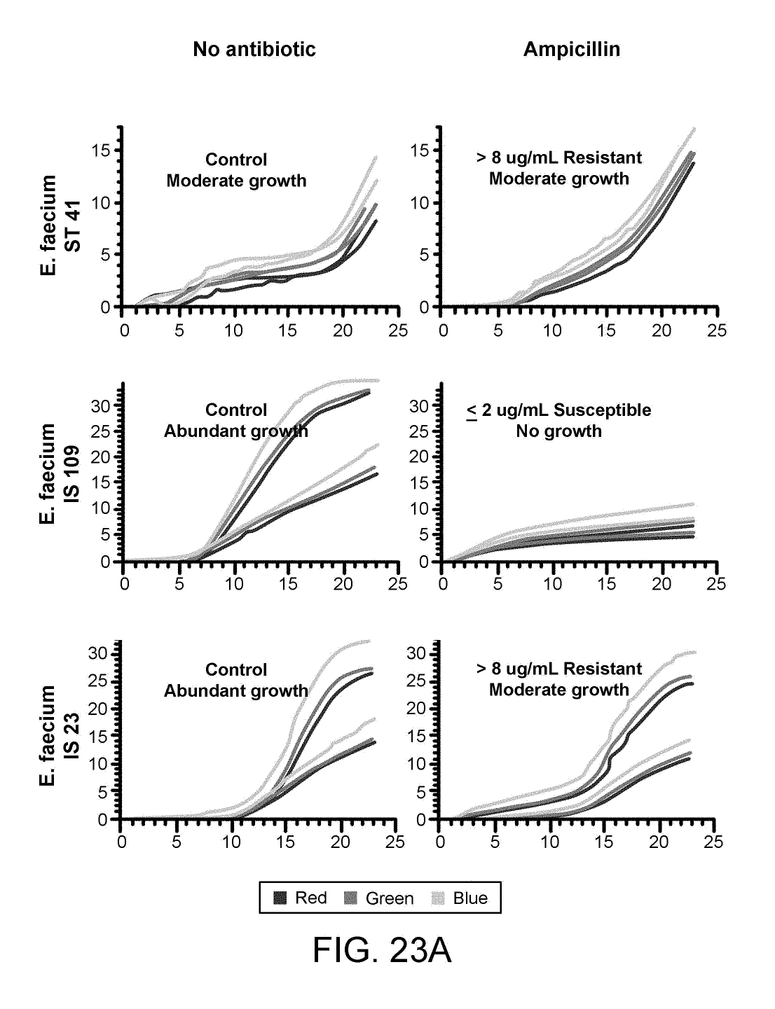

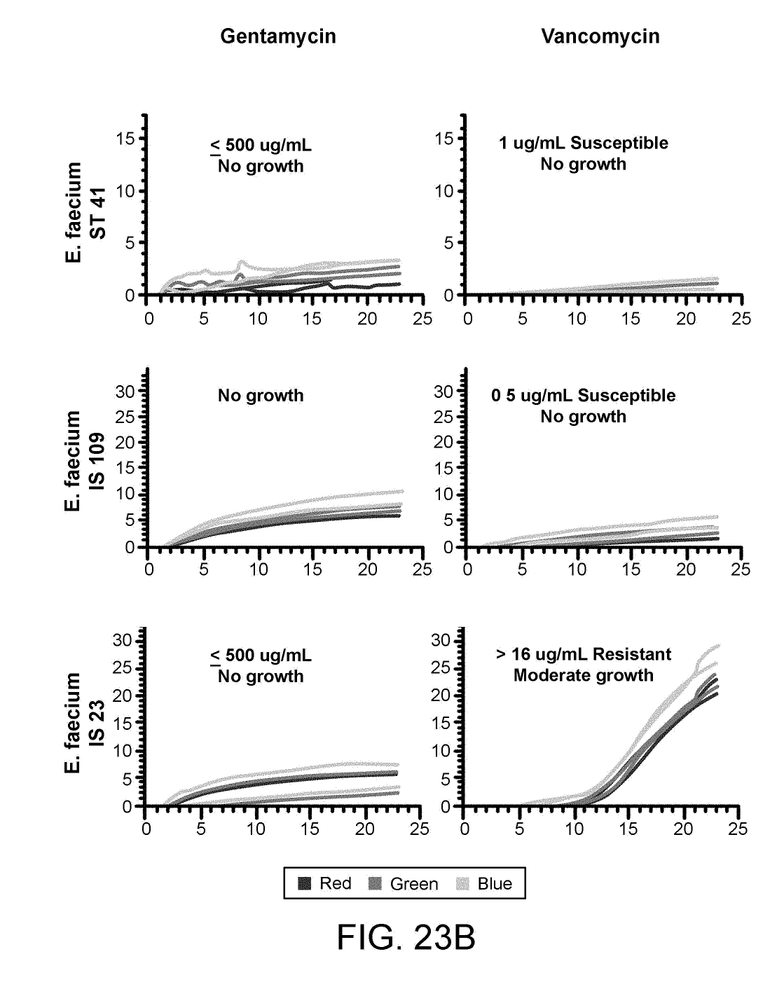

FIGS. 13A-23B show temporal results of susceptibility tests for various sensors of a colorimetric sensor array for identified strains of E. faecium.

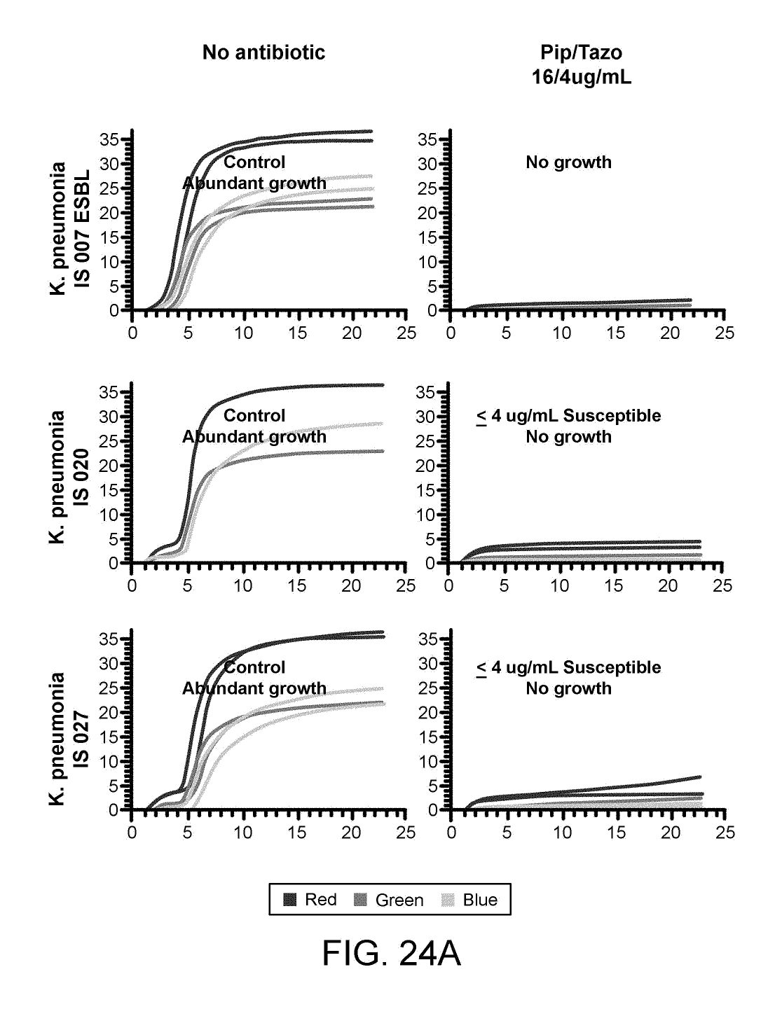

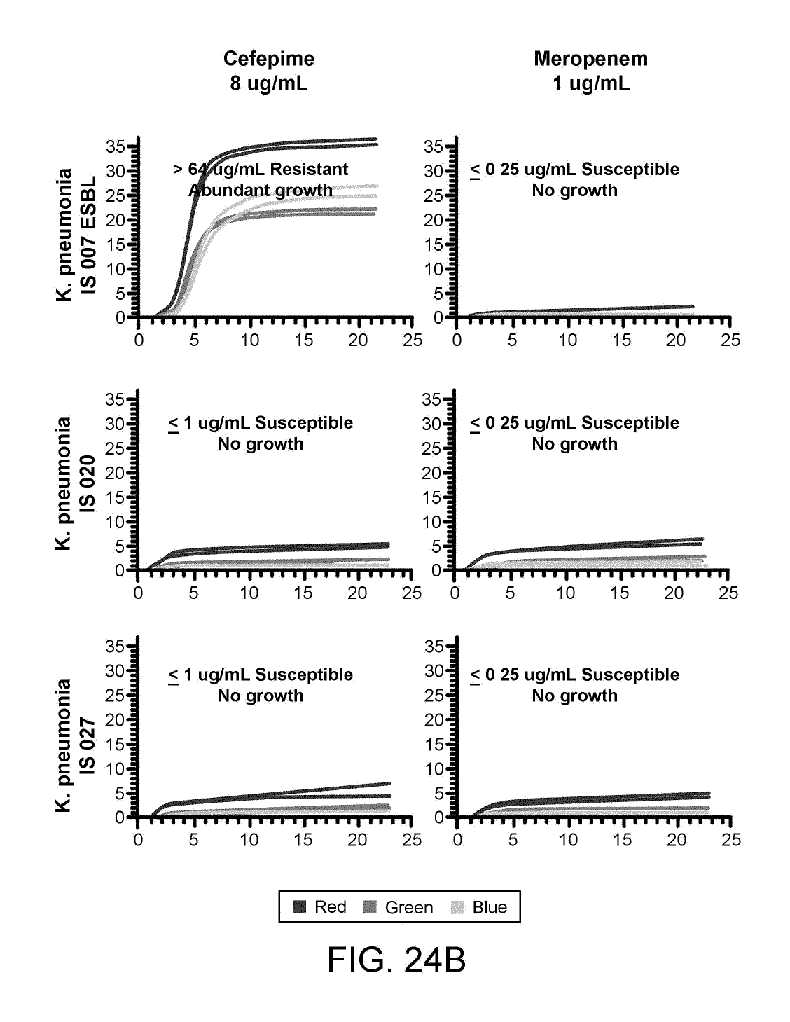

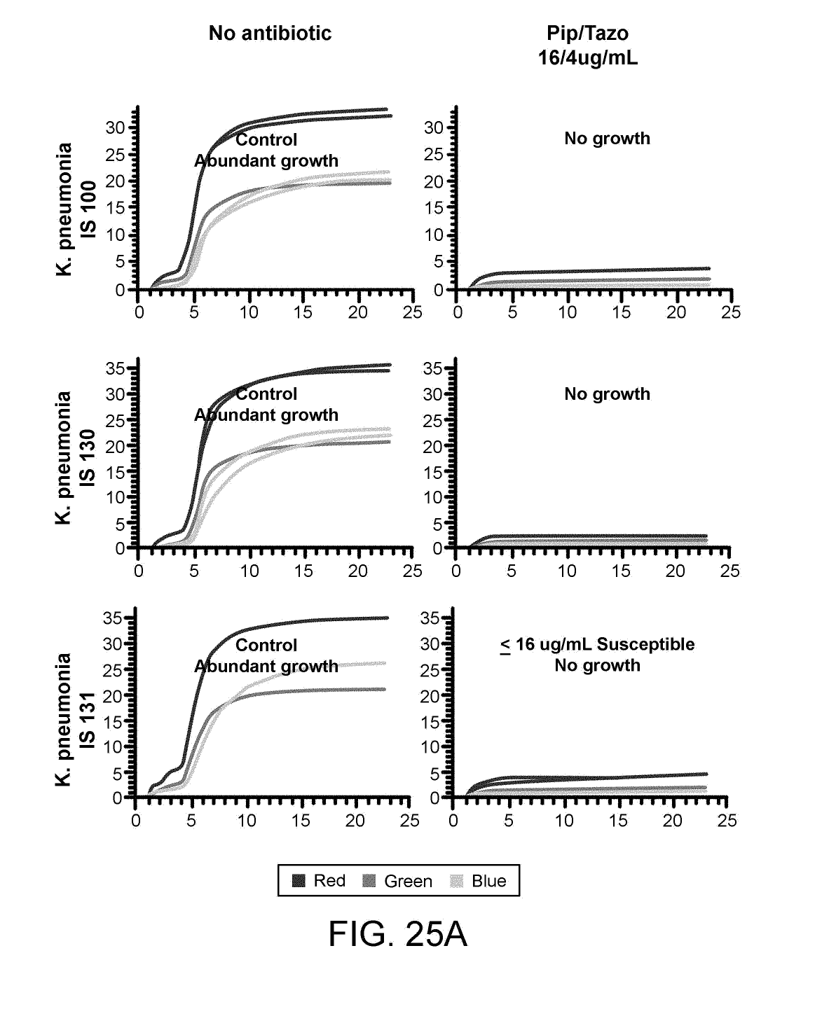

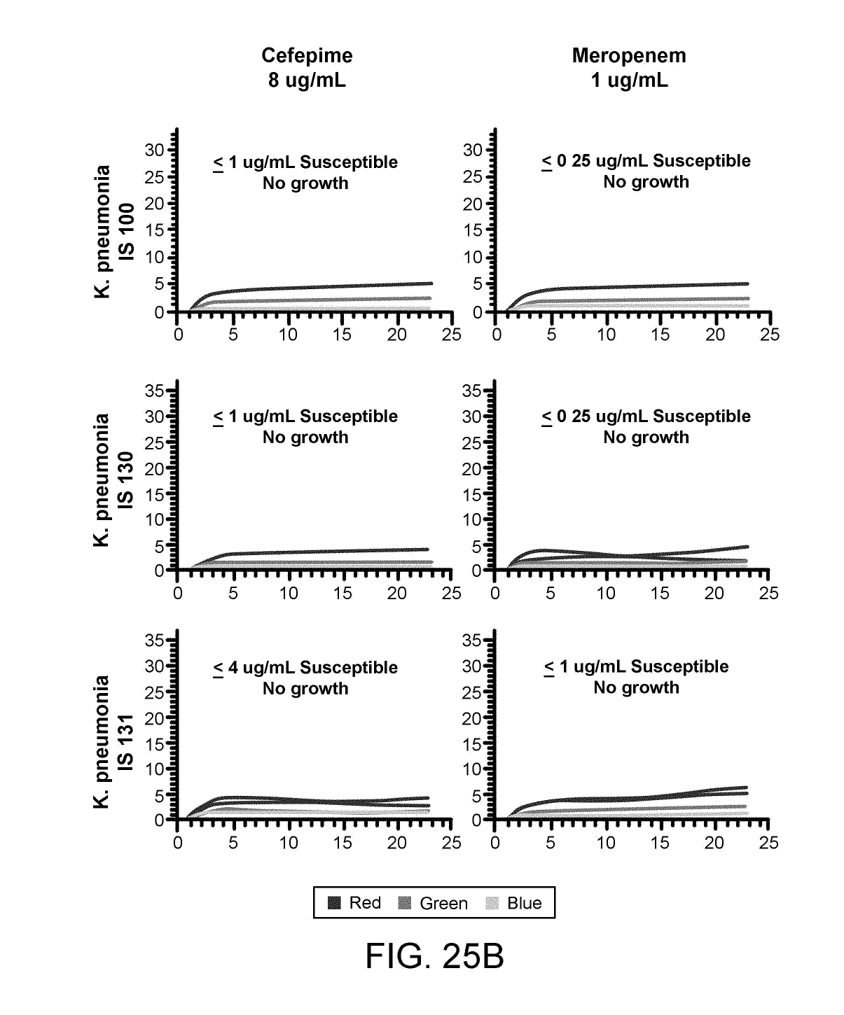

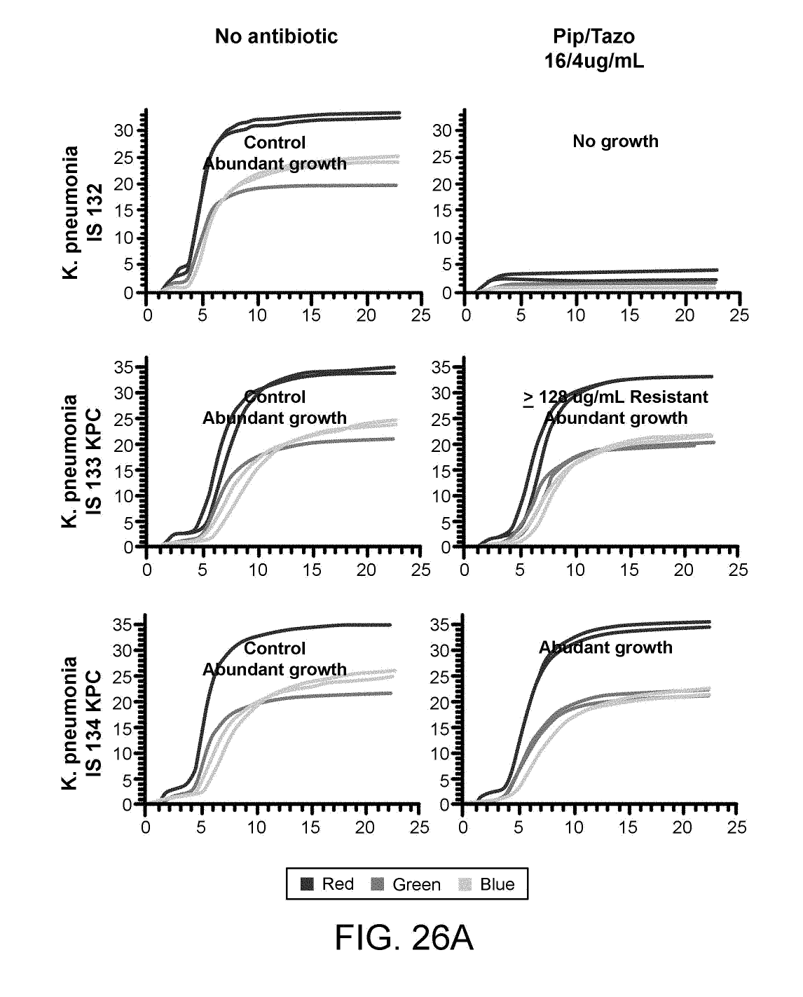

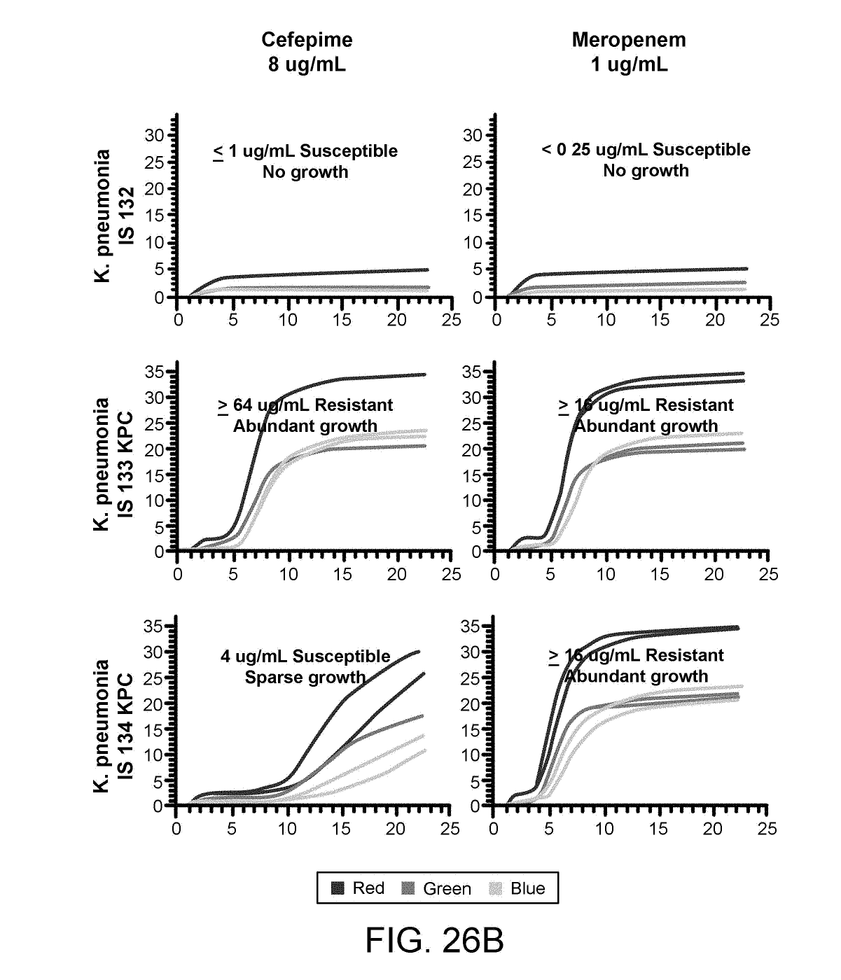

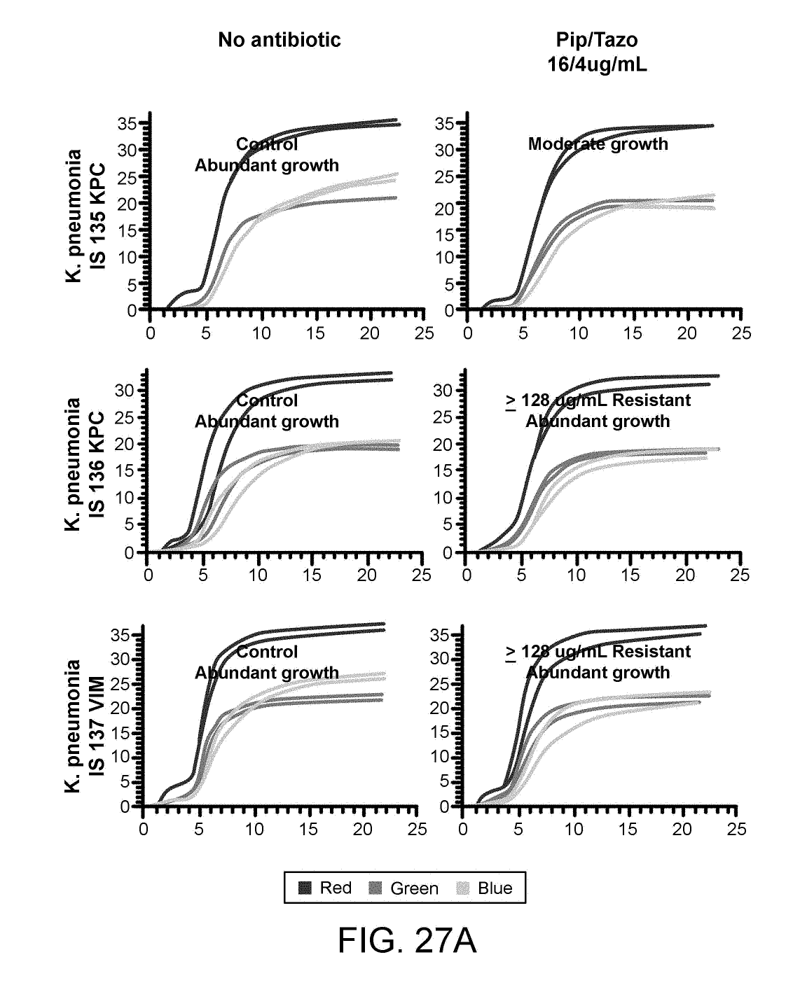

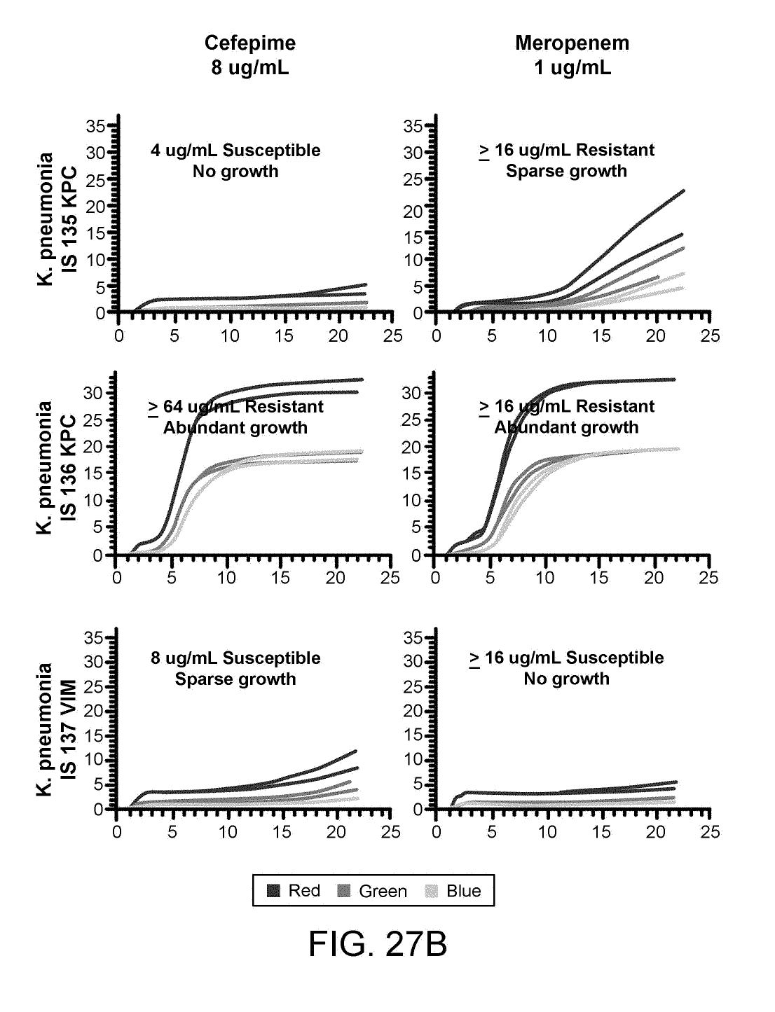

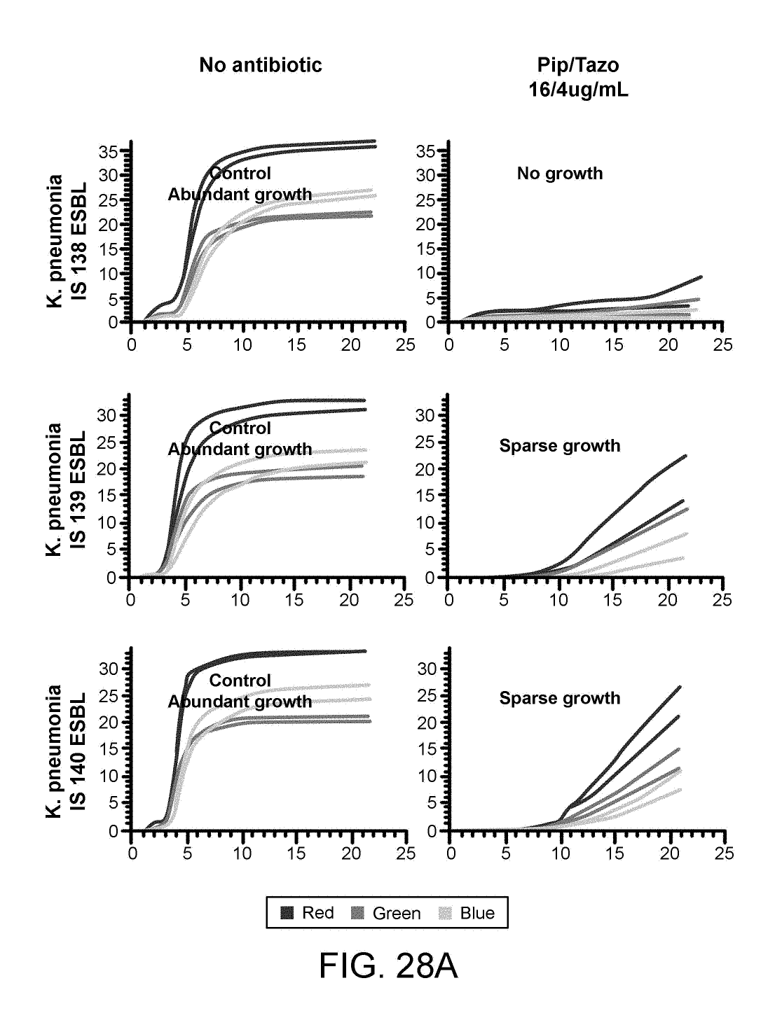

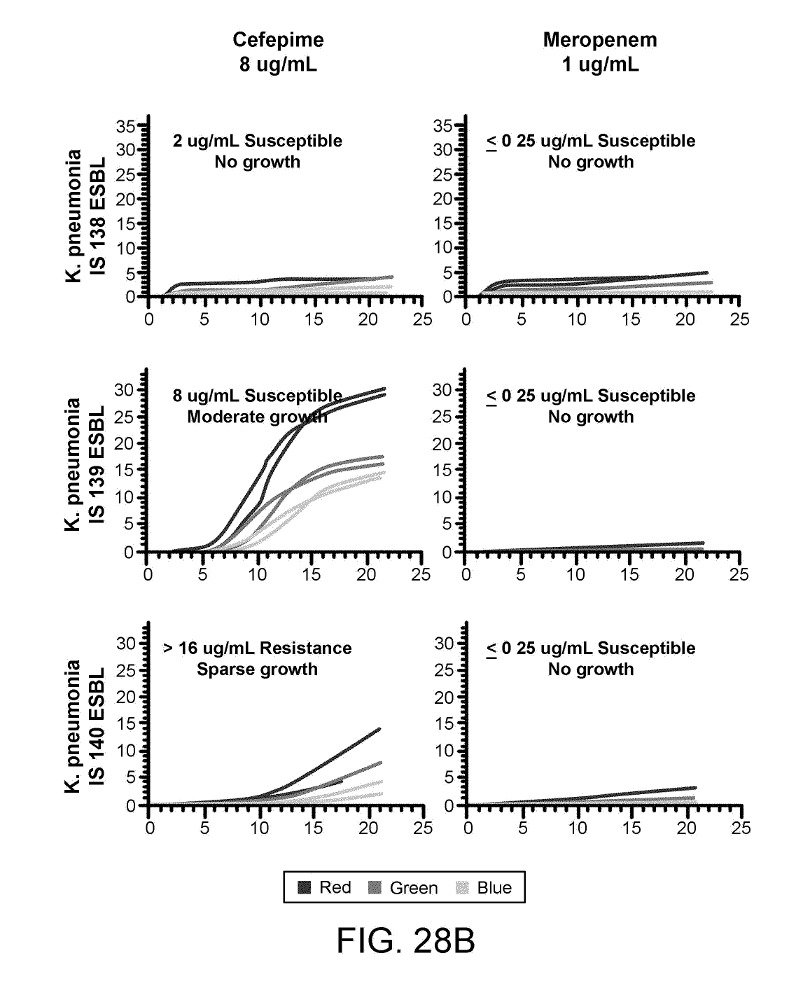

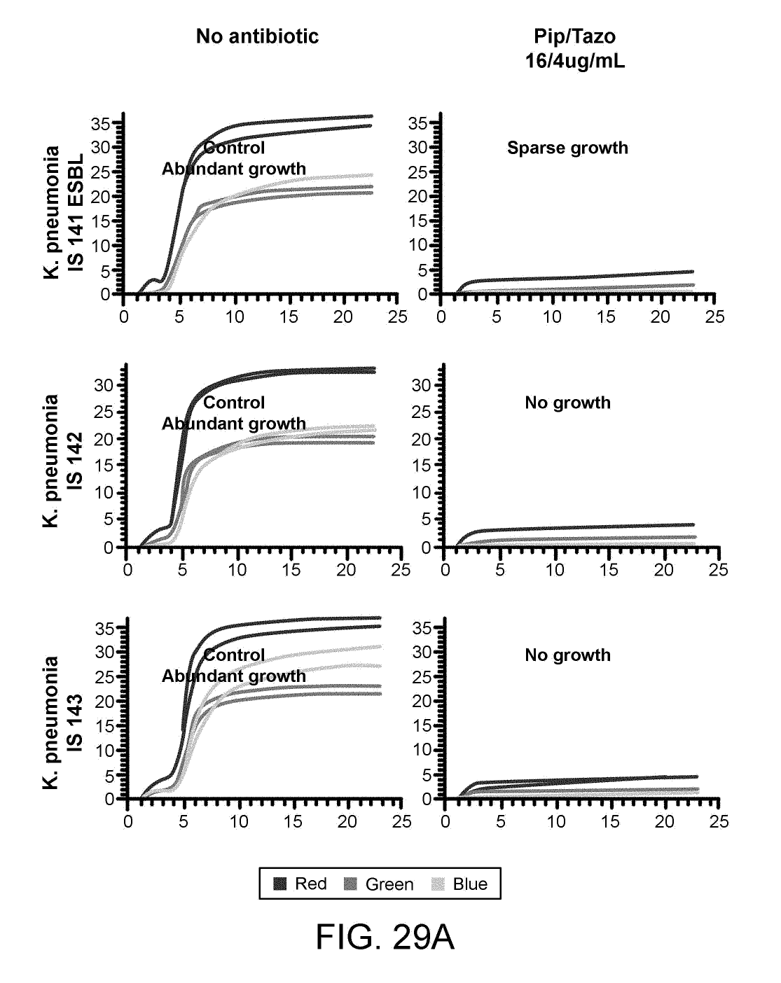

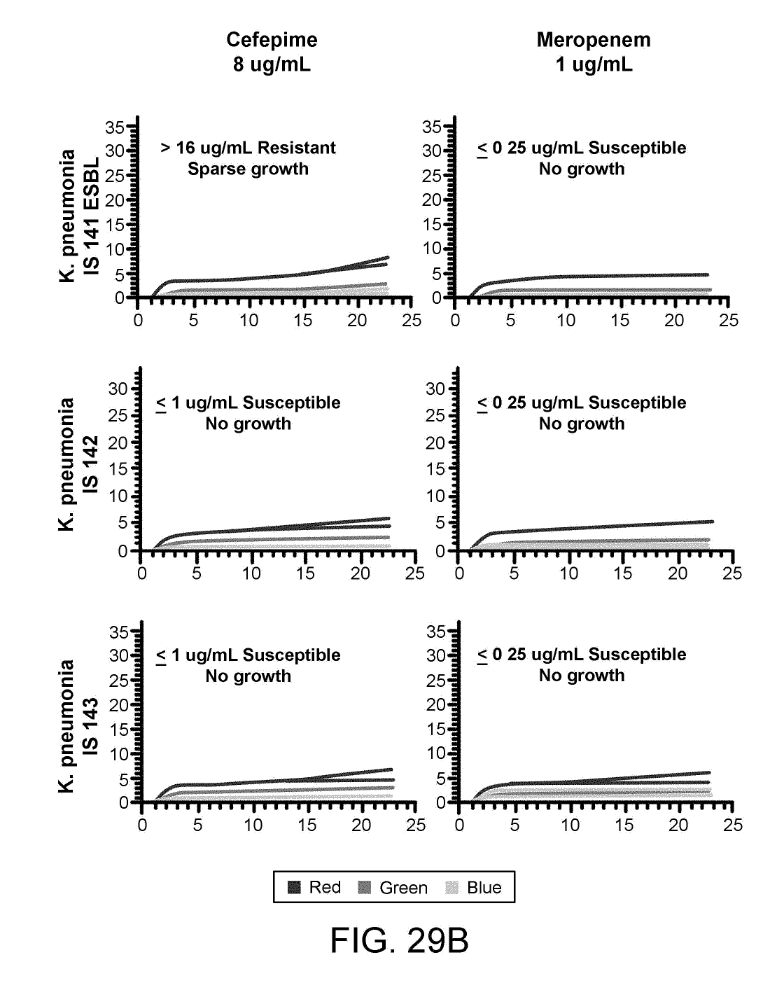

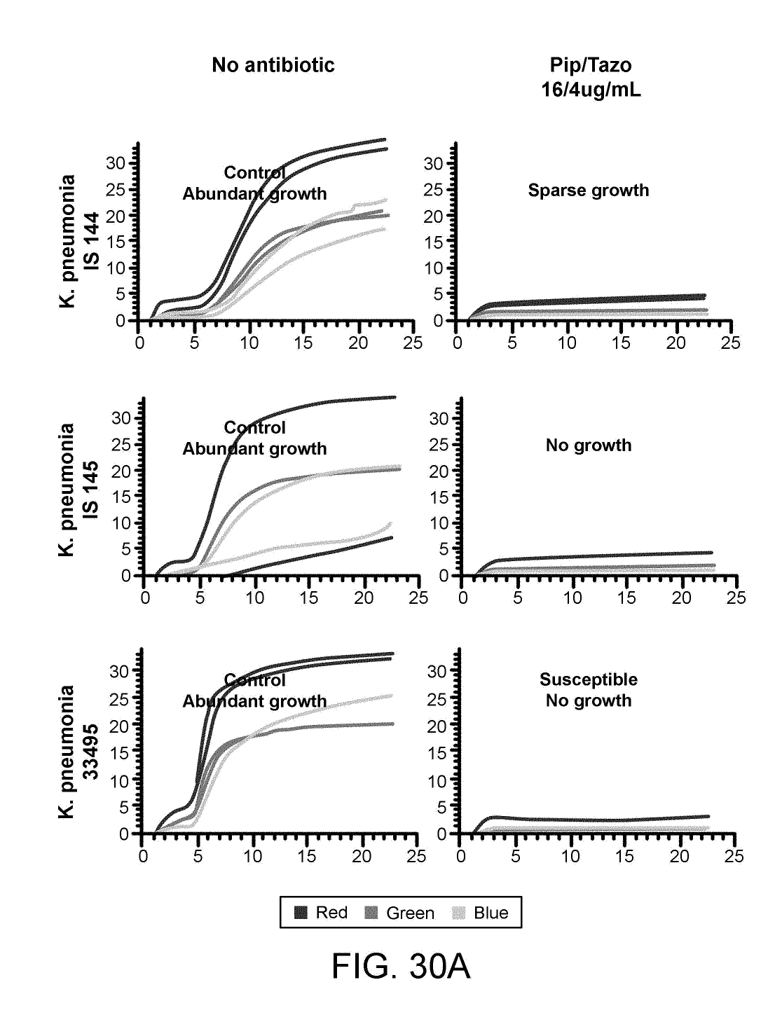

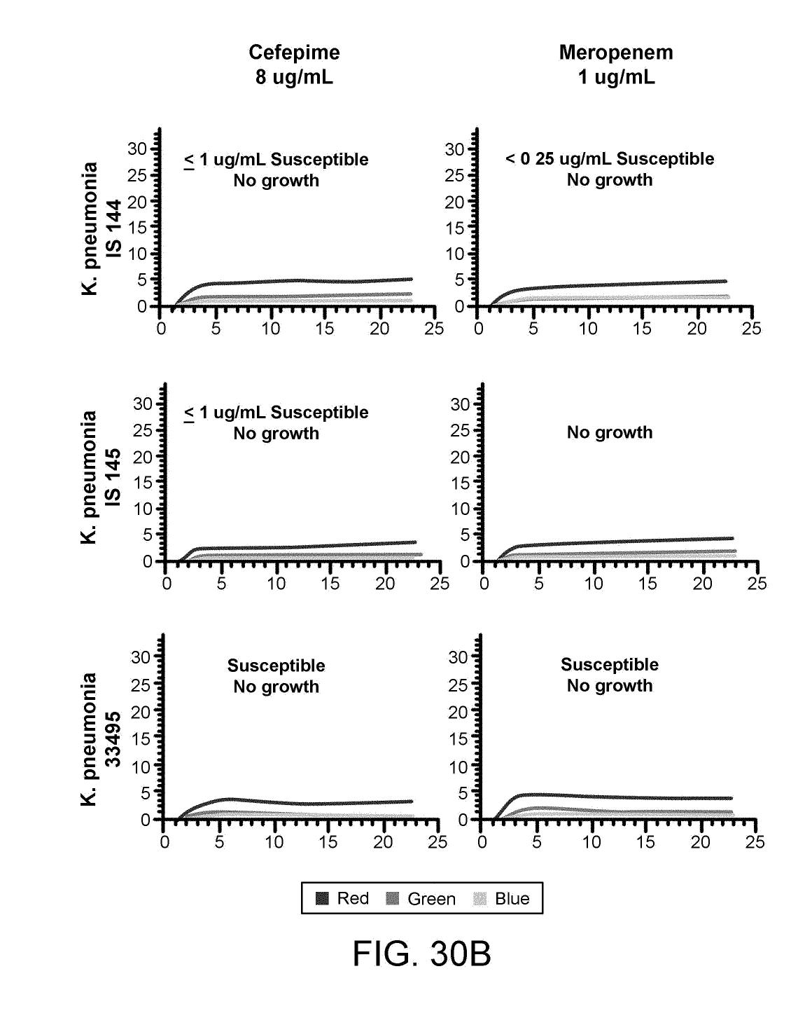

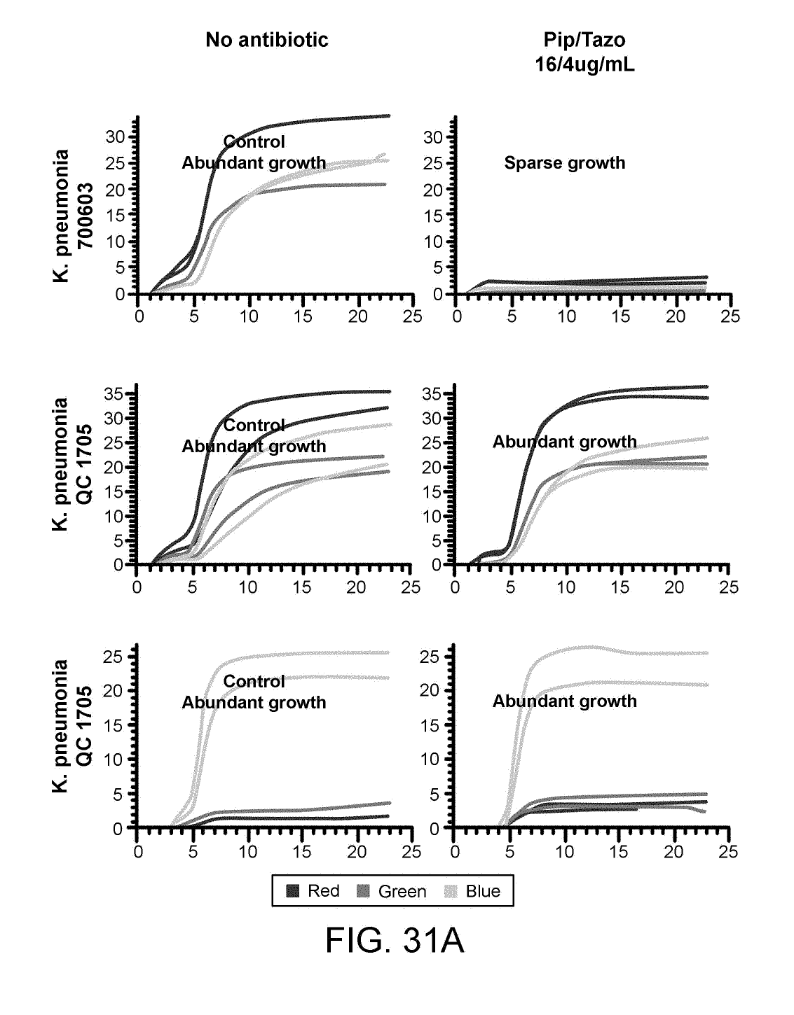

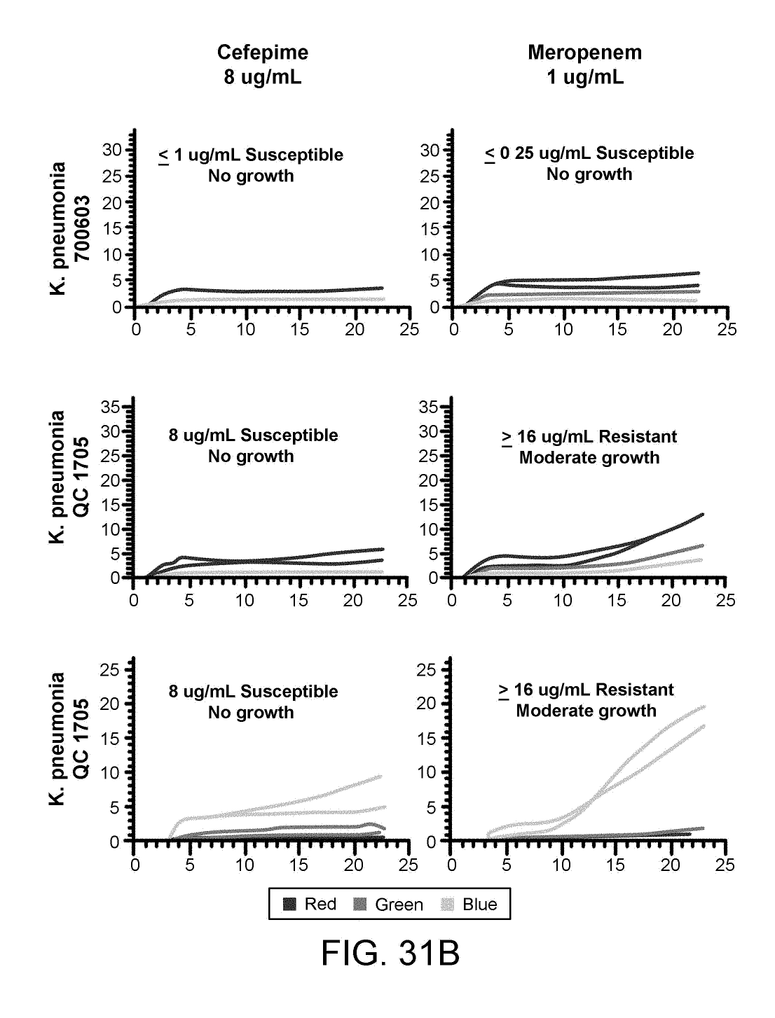

FIGS. 24A-31B show temporal results of susceptibility tests for various sensors of a colorimetric sensor array for identified strains of K. pneumoniae.

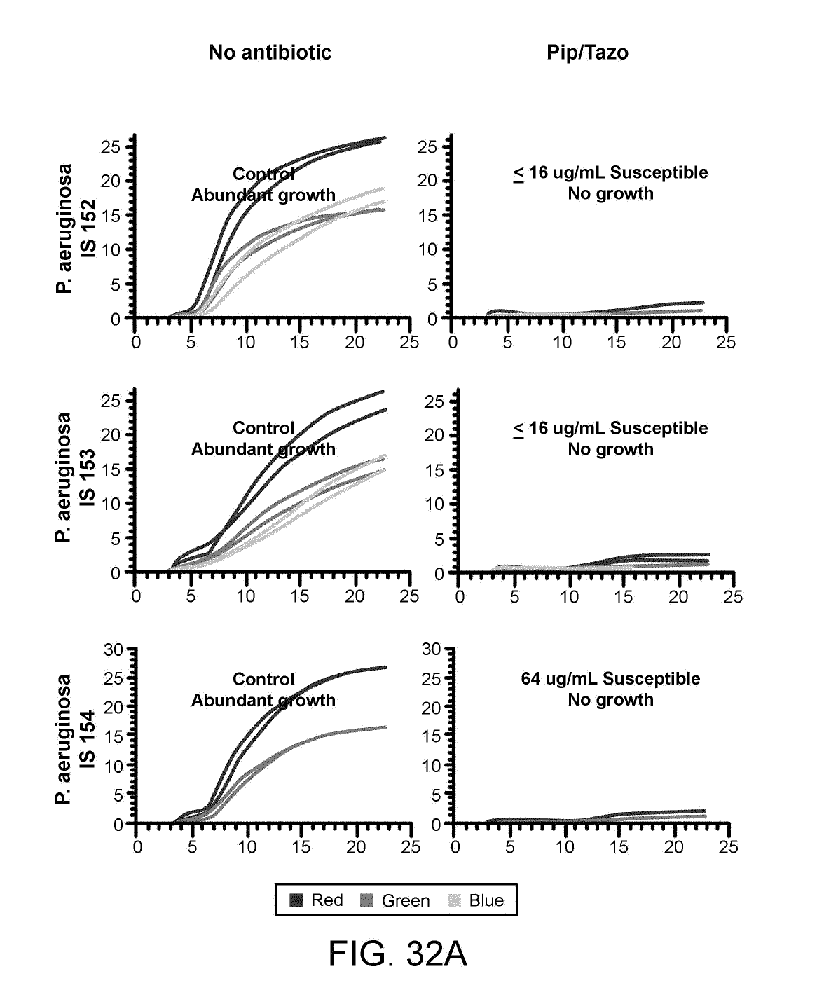

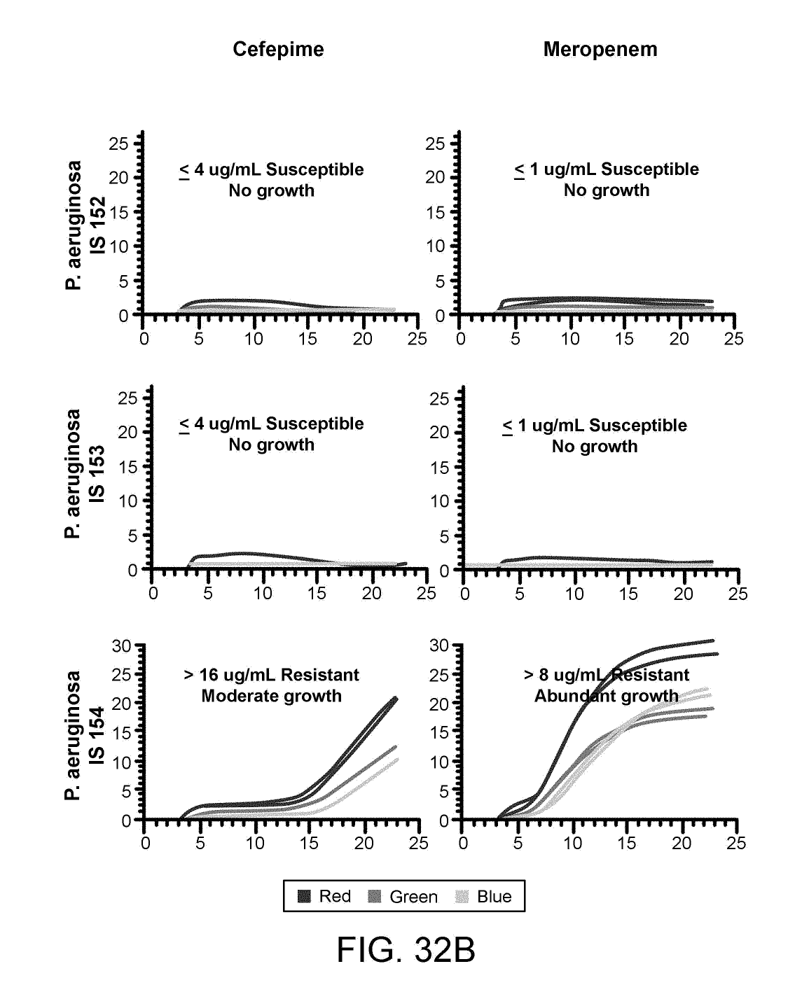

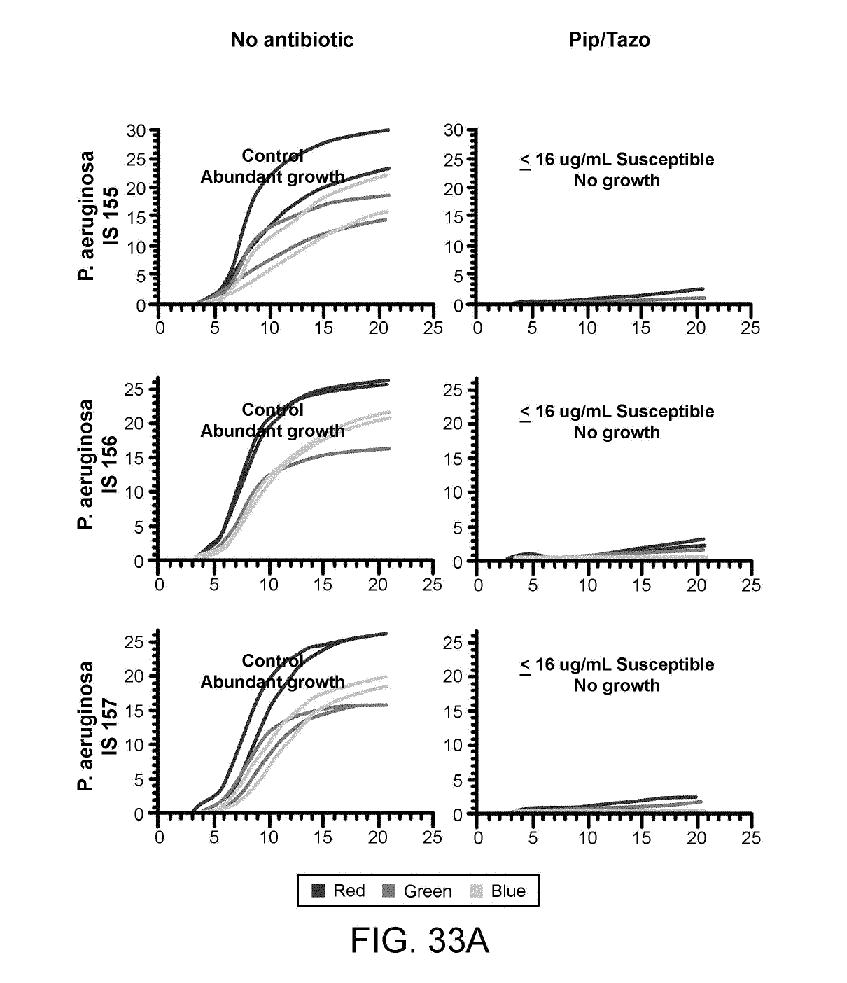

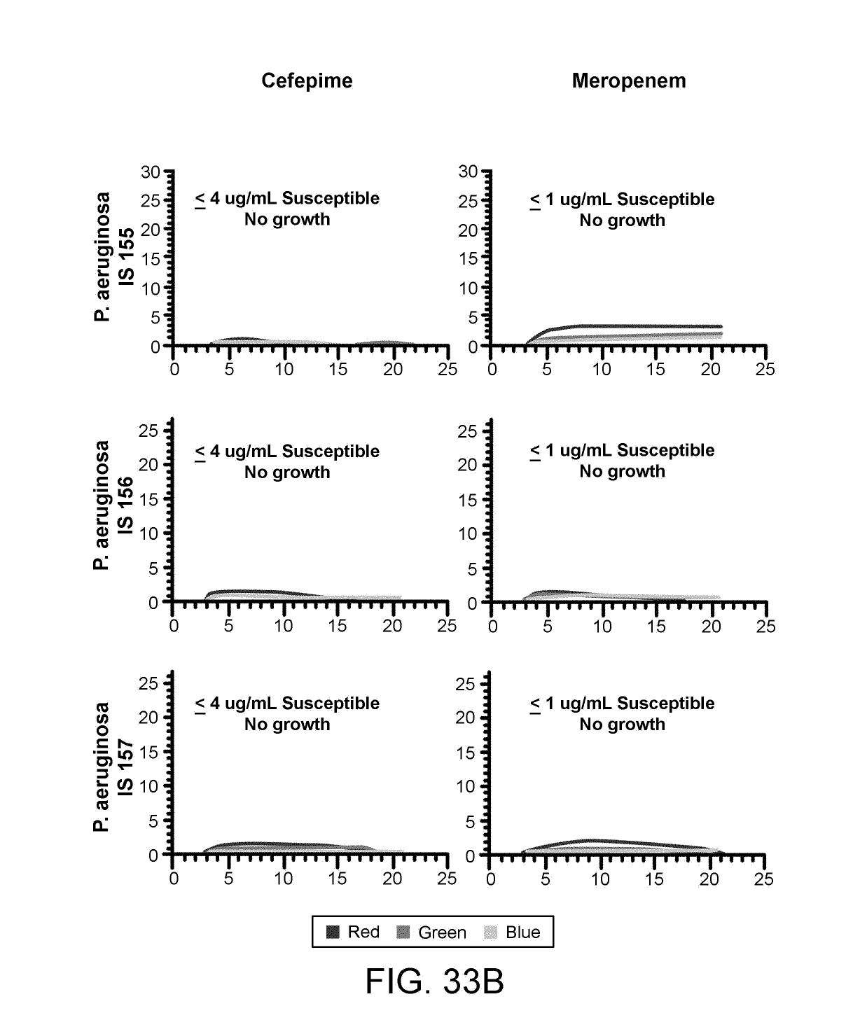

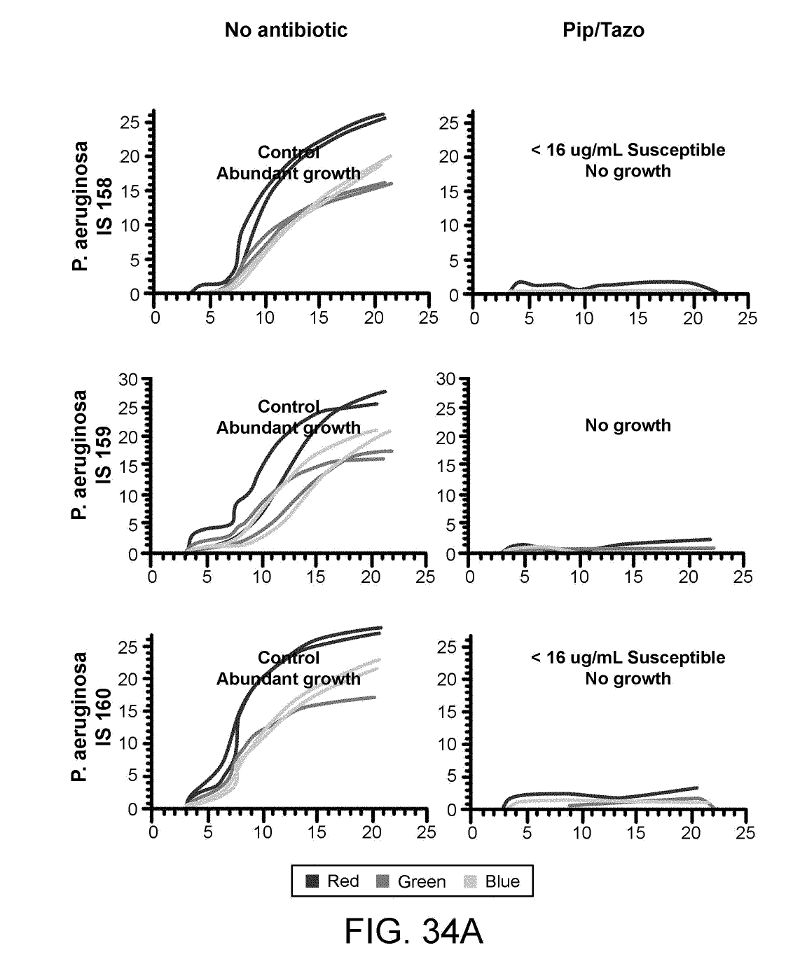

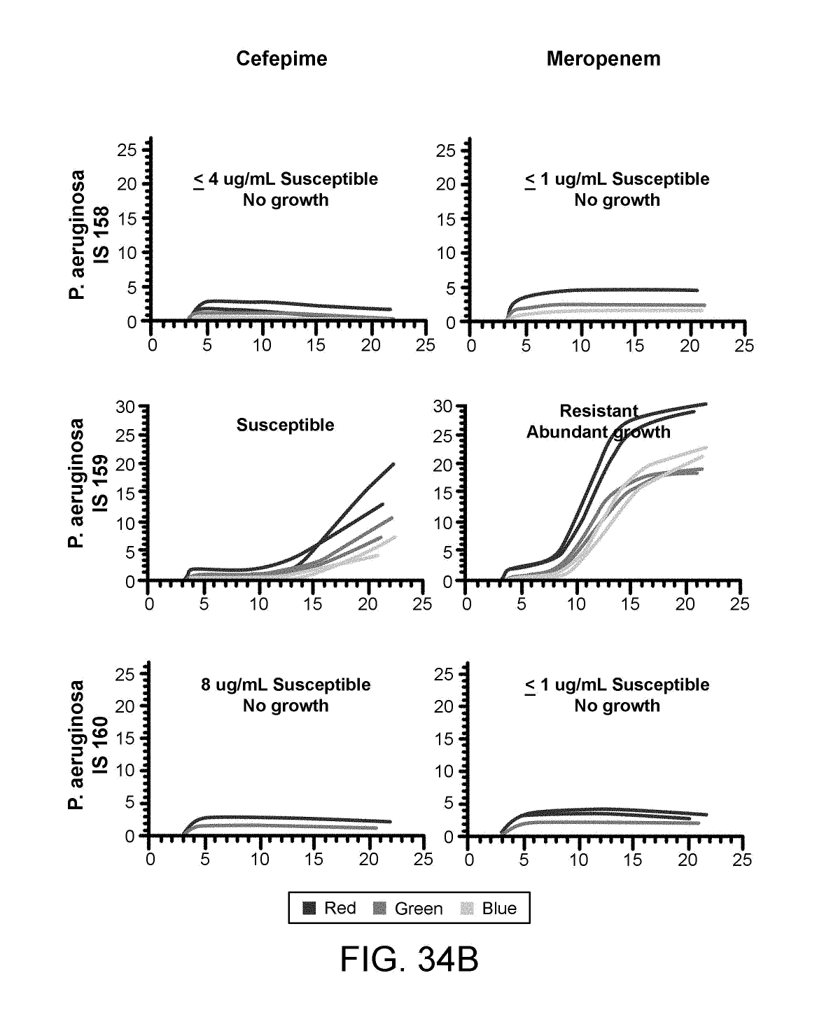

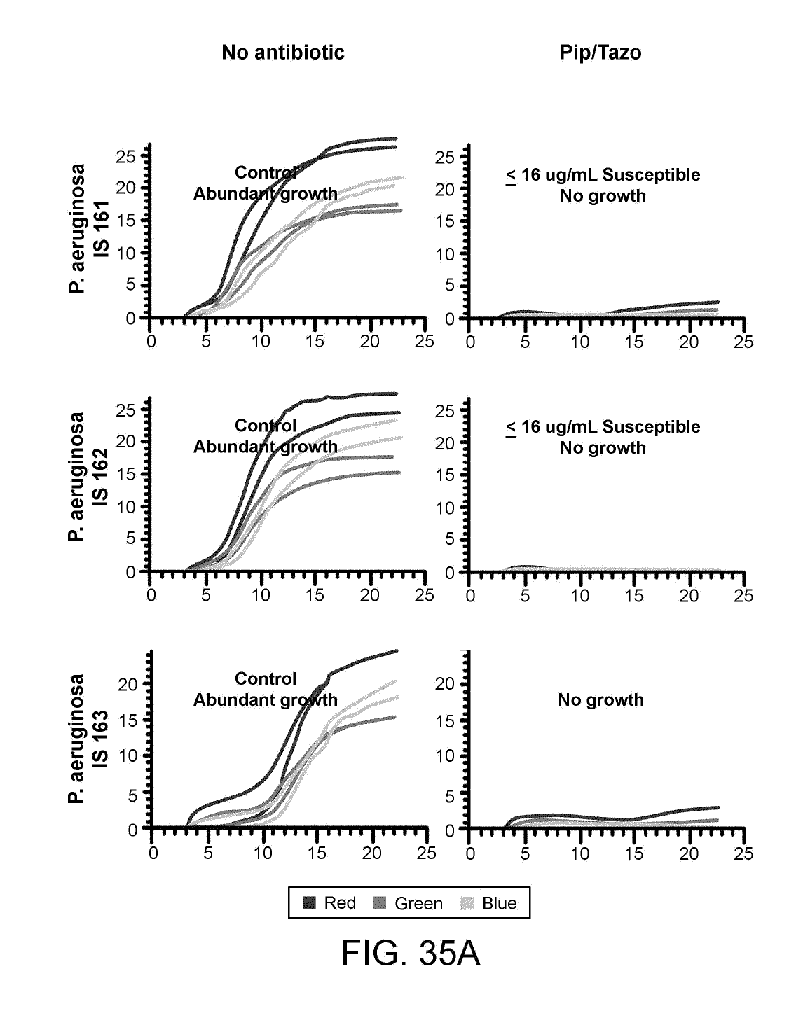

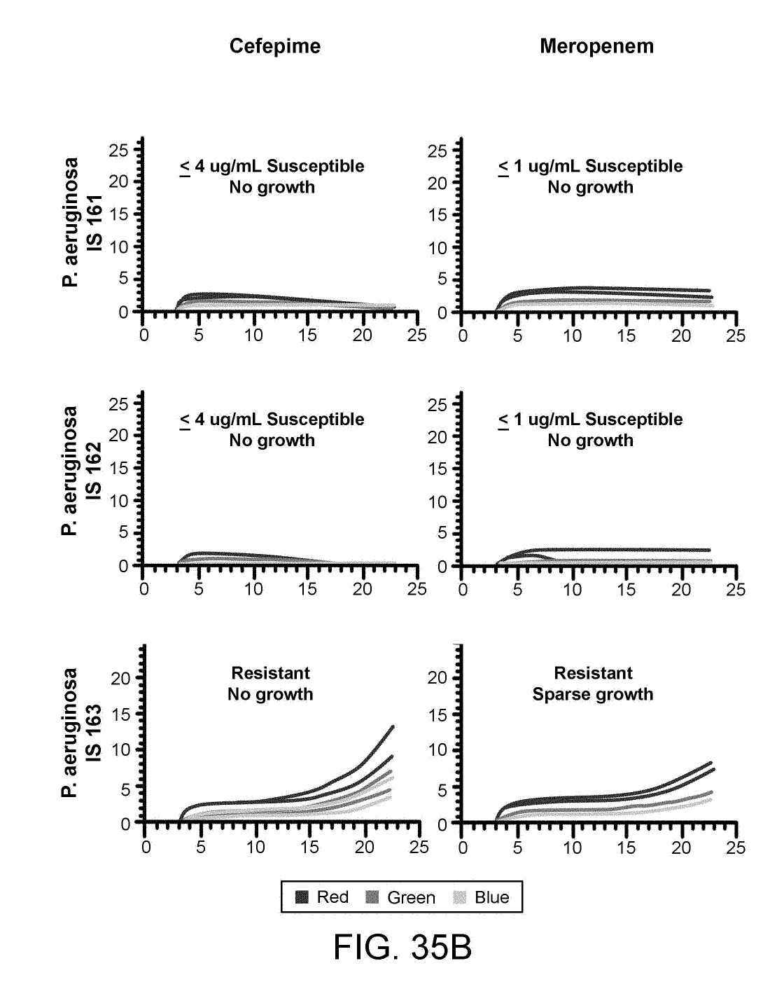

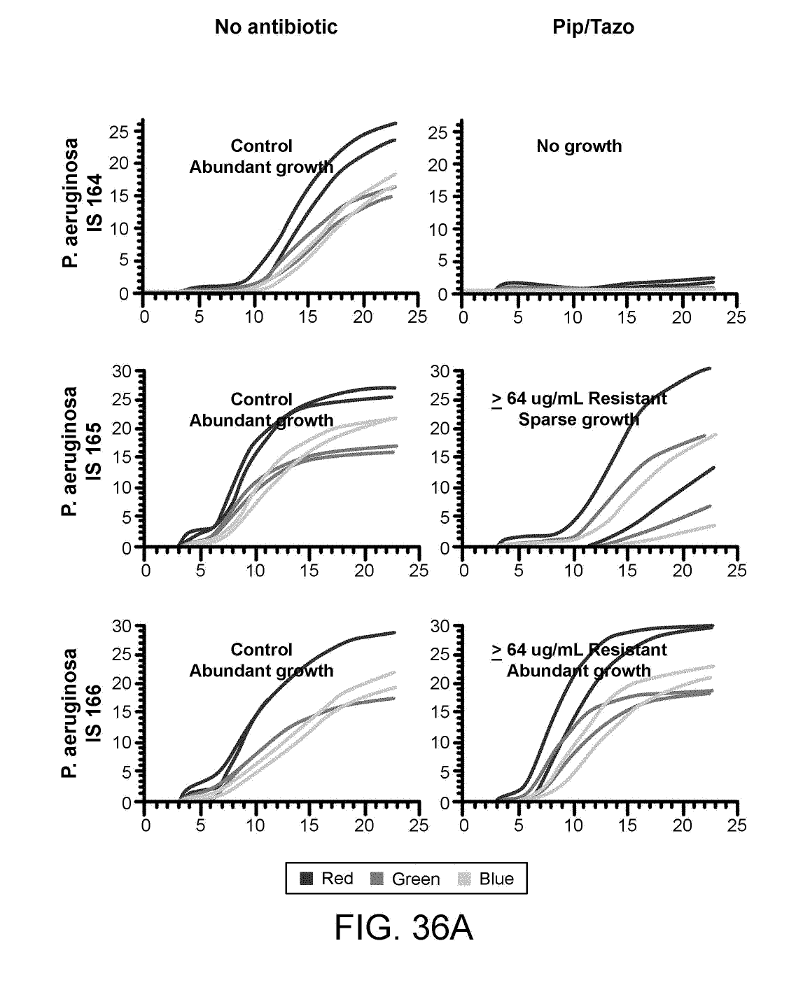

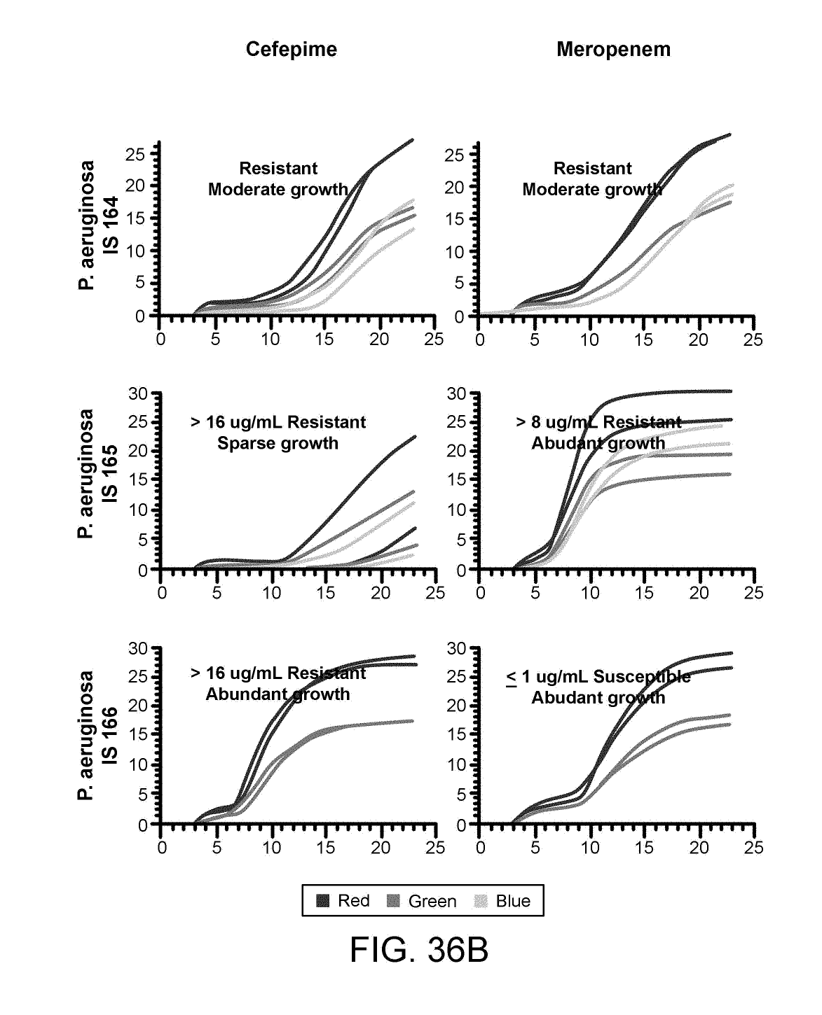

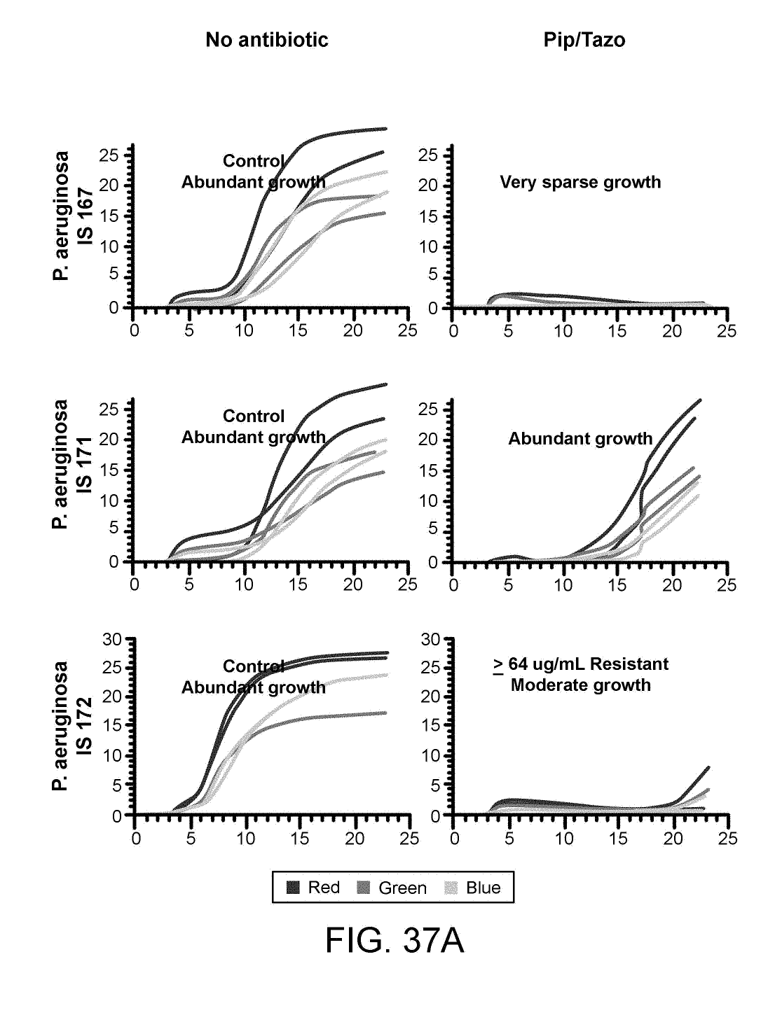

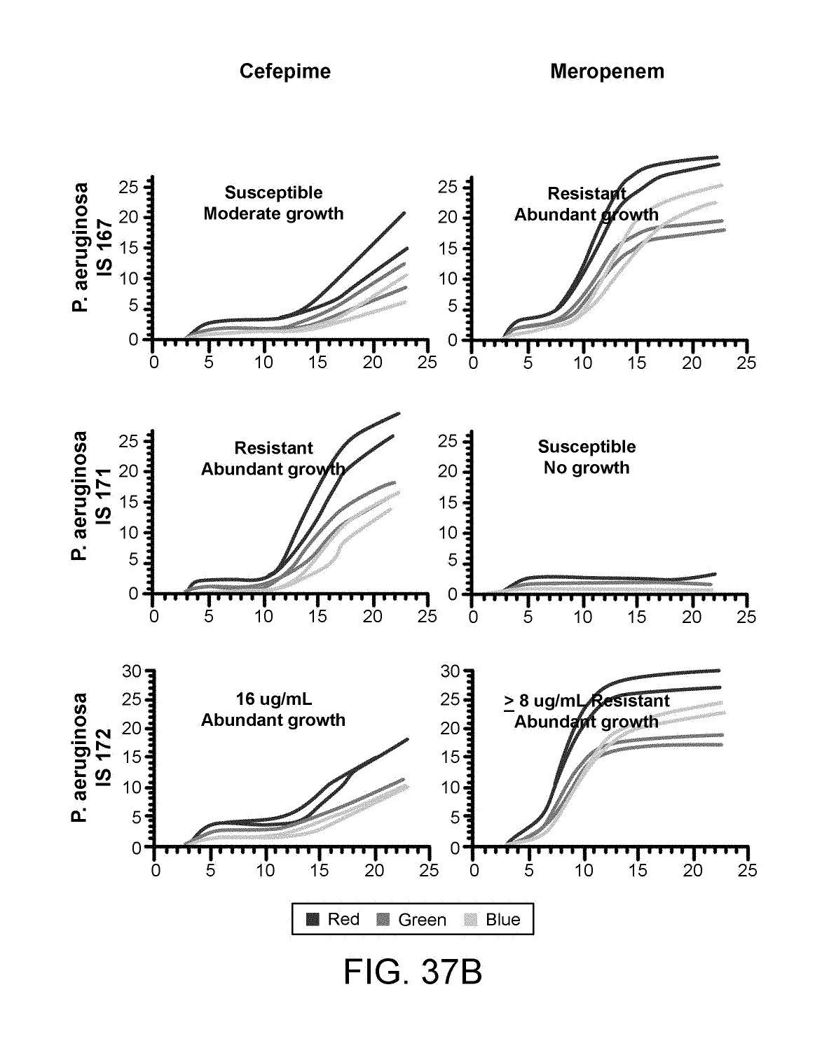

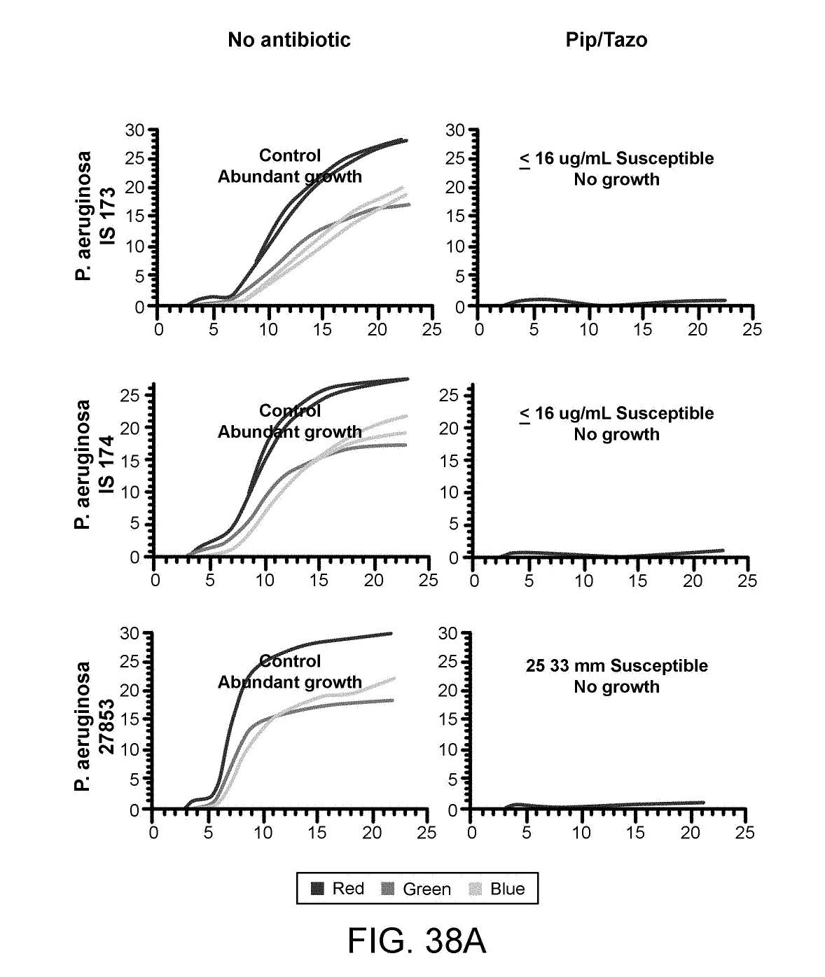

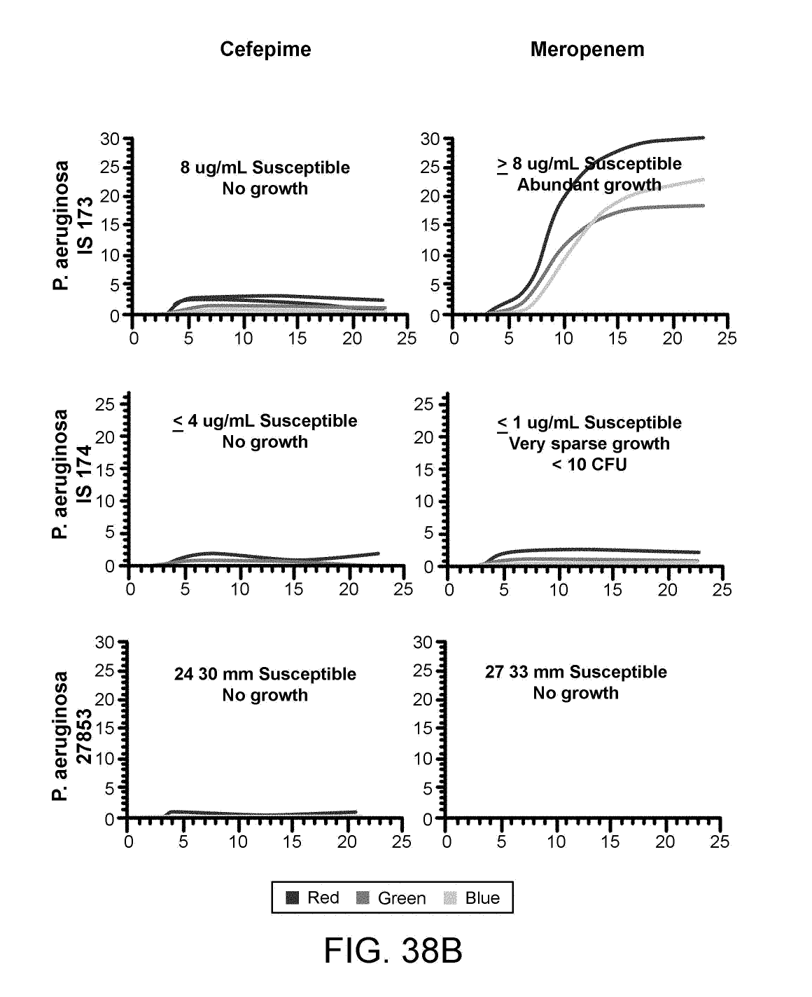

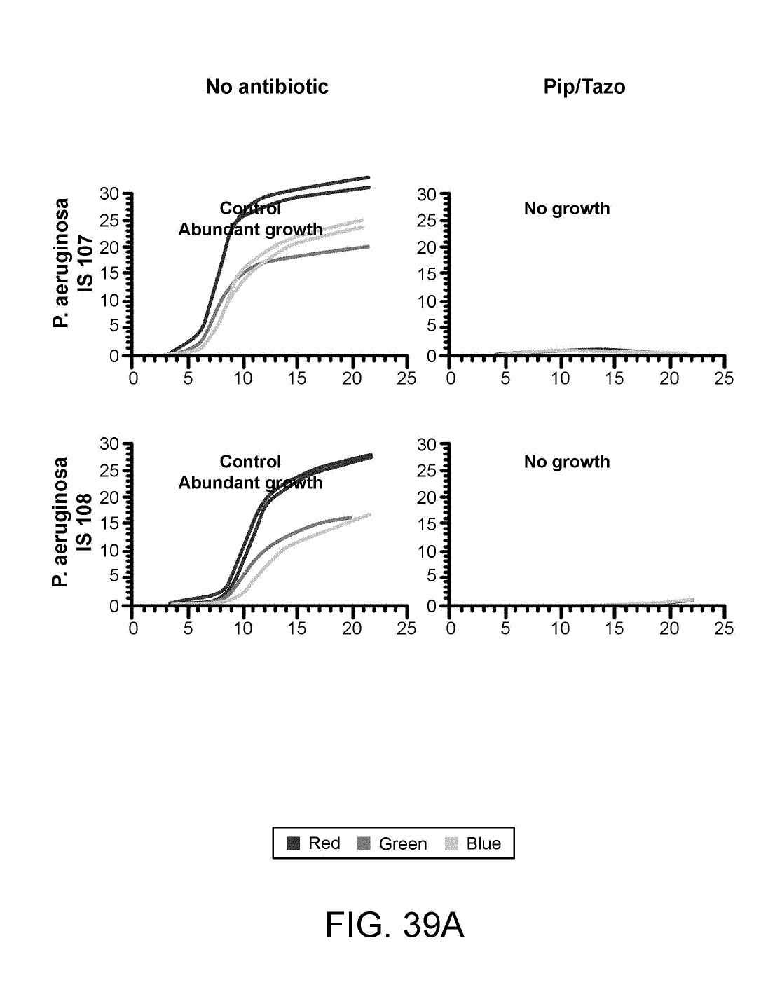

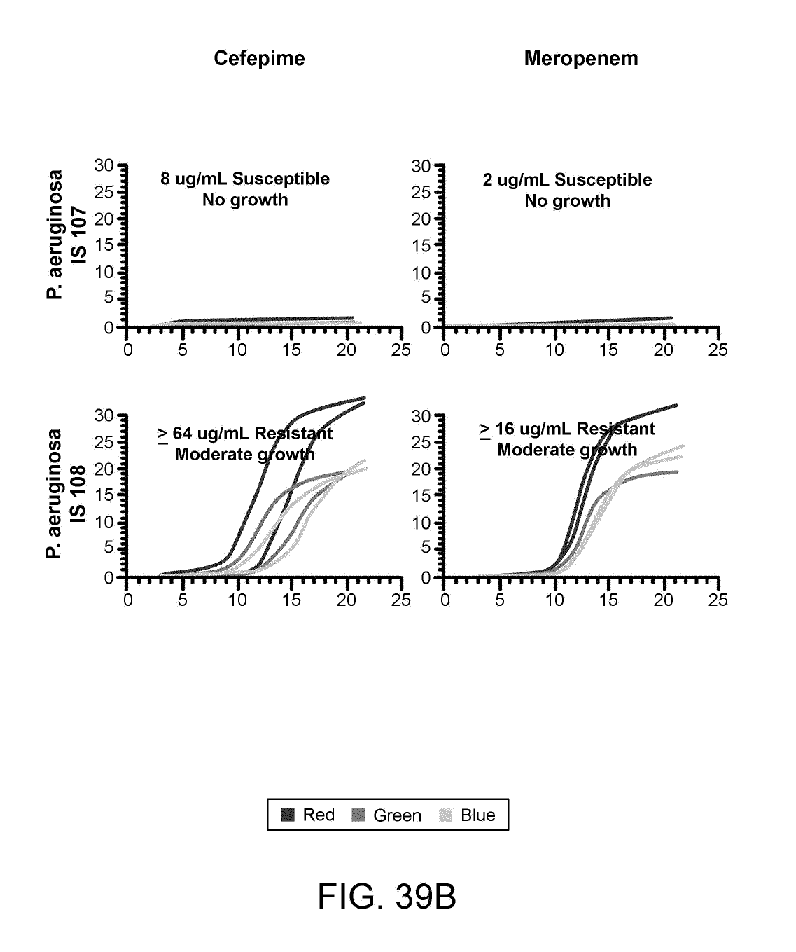

FIGS. 32A-39B show temporal results of susceptibility tests for various sensors of a colorimetric sensor array for identified strains of P. aeruginosa.

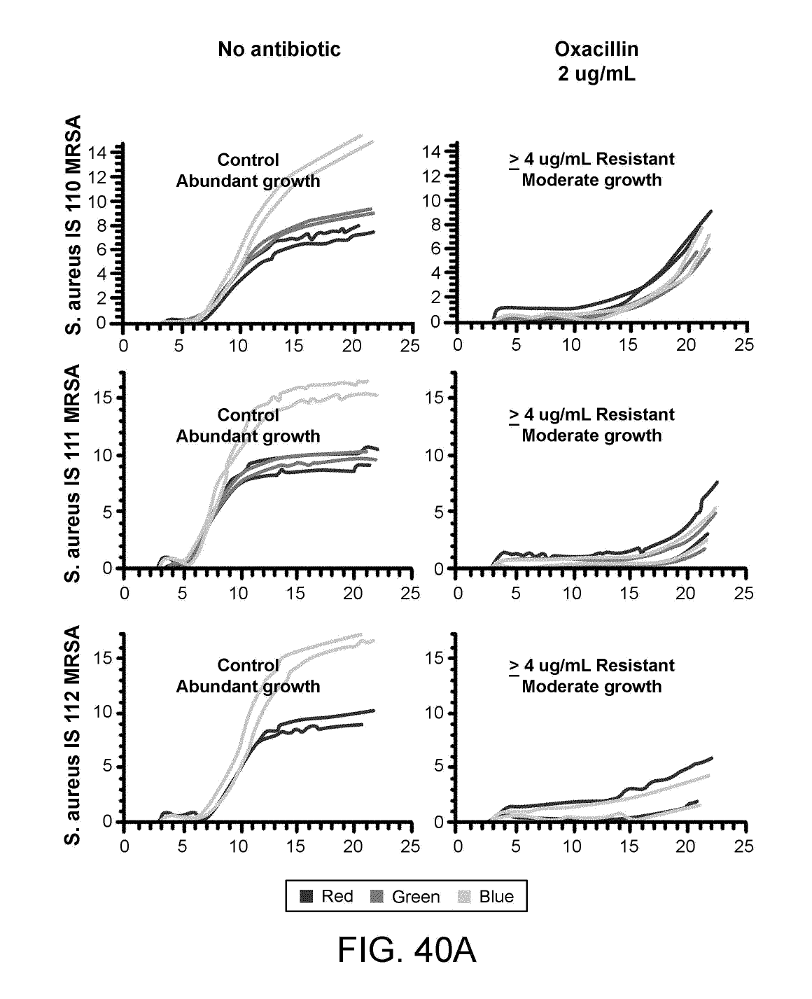

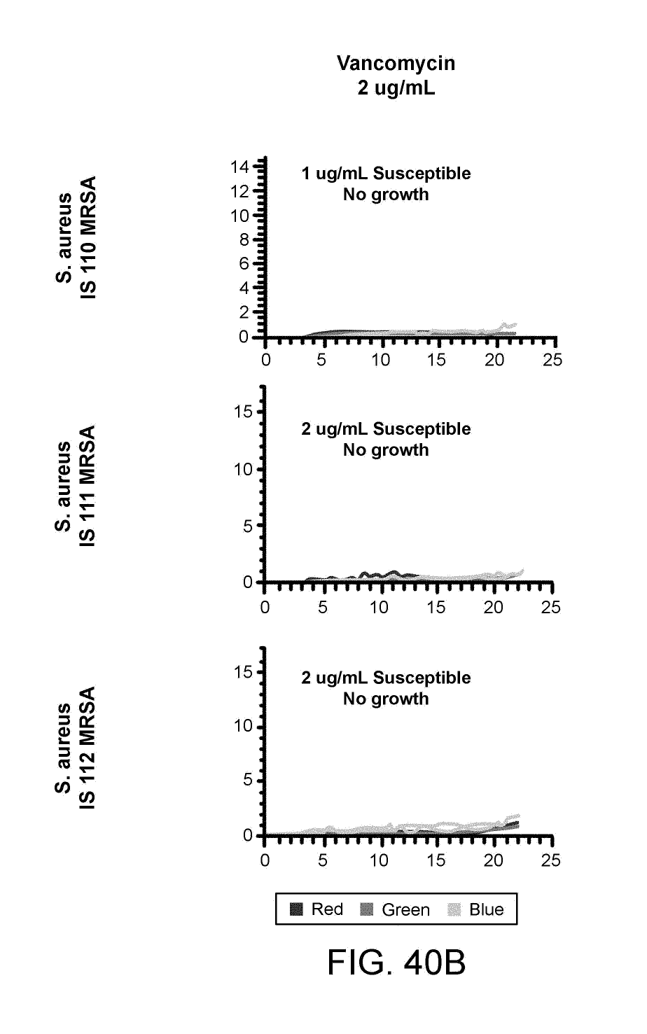

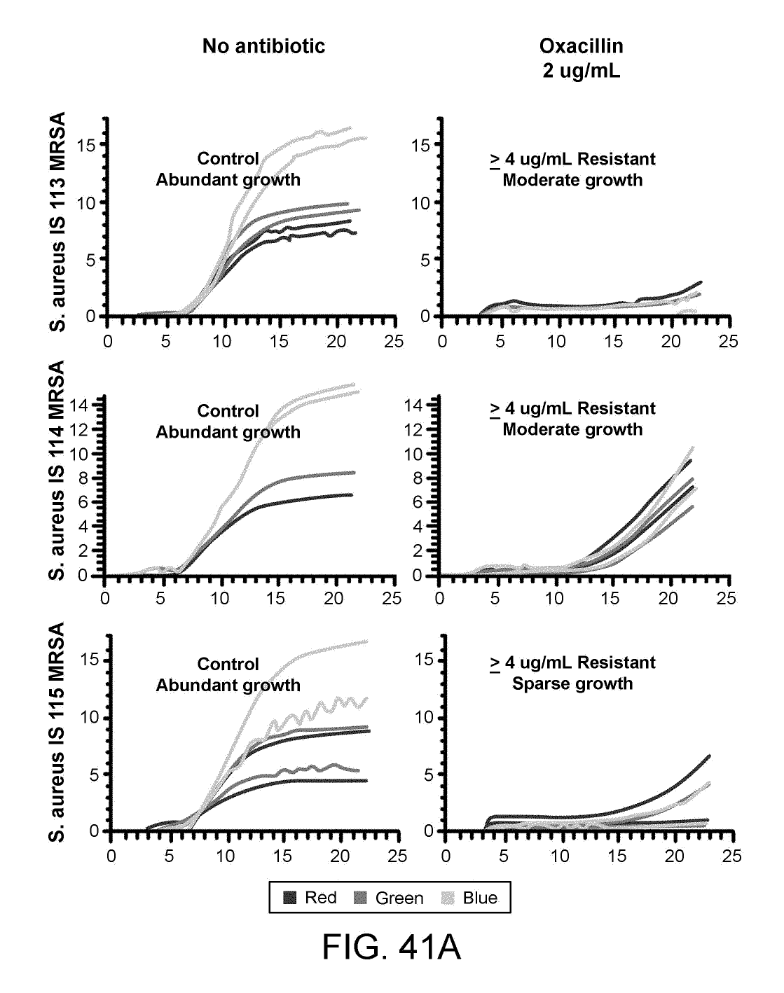

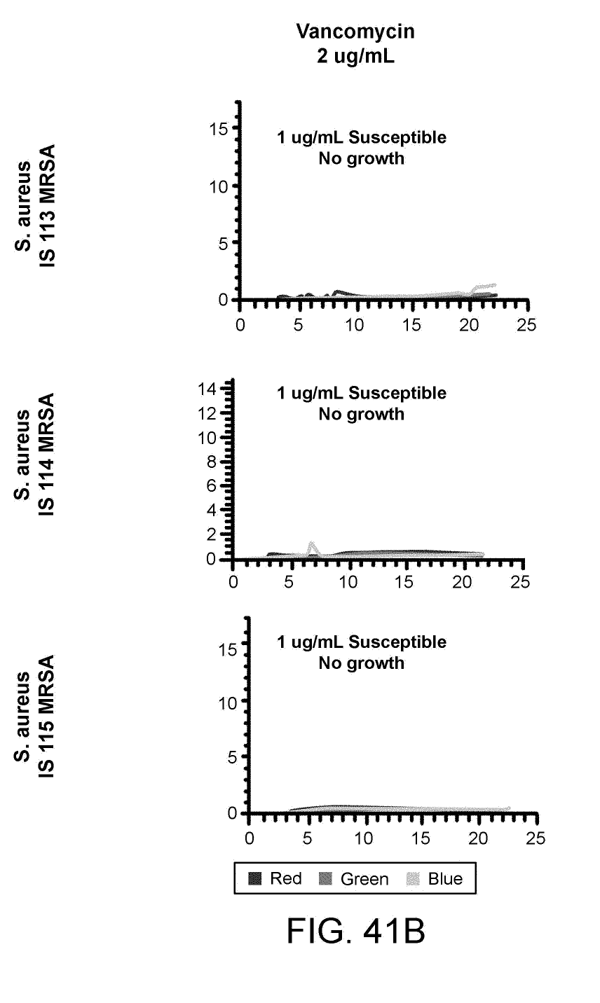

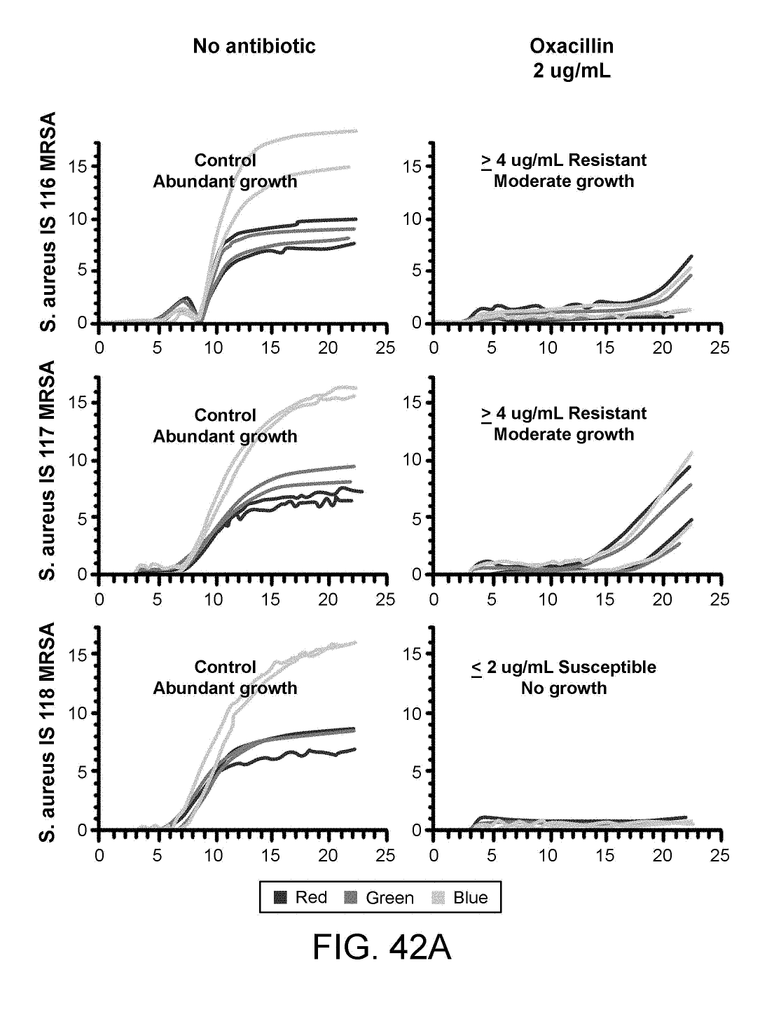

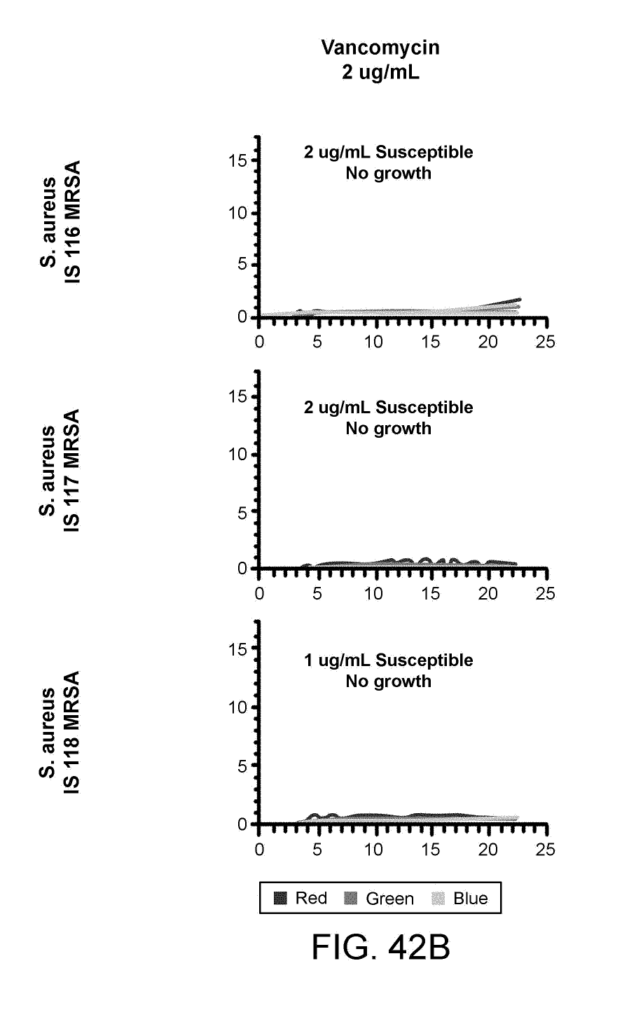

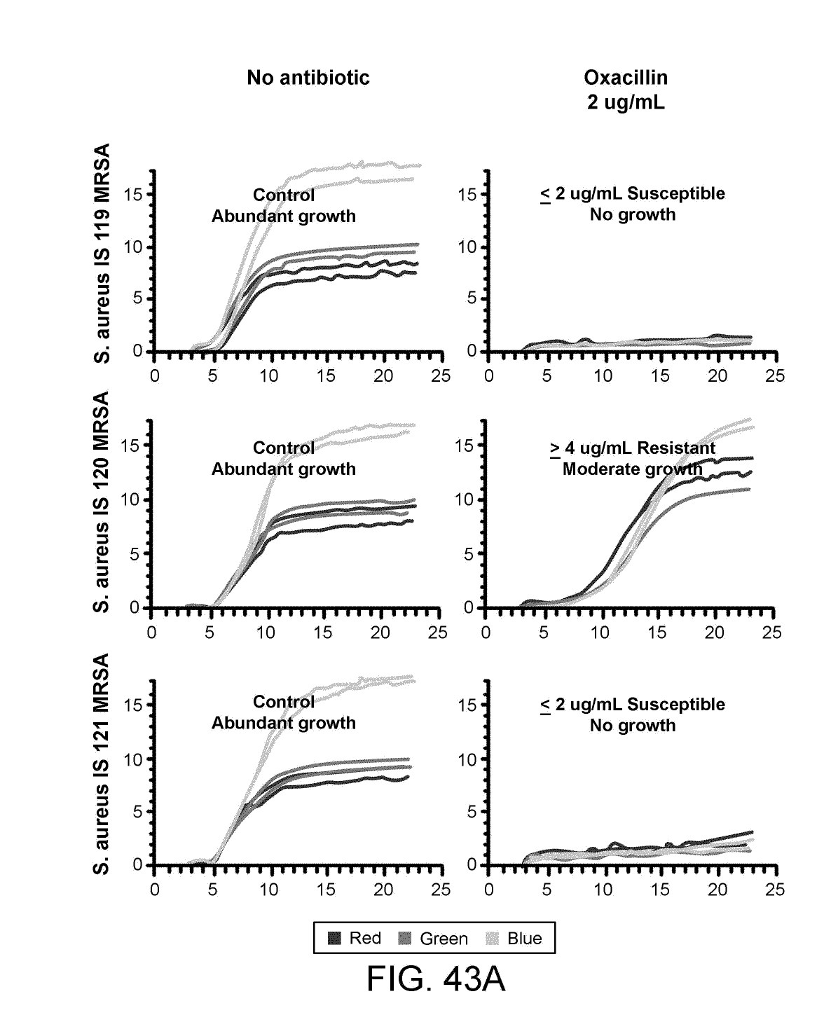

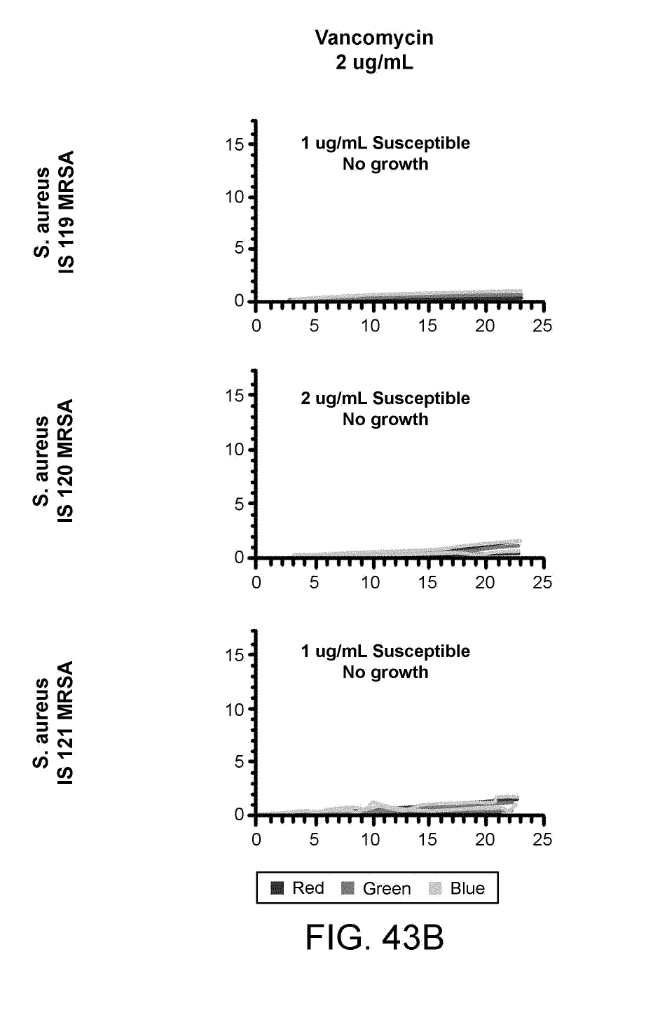

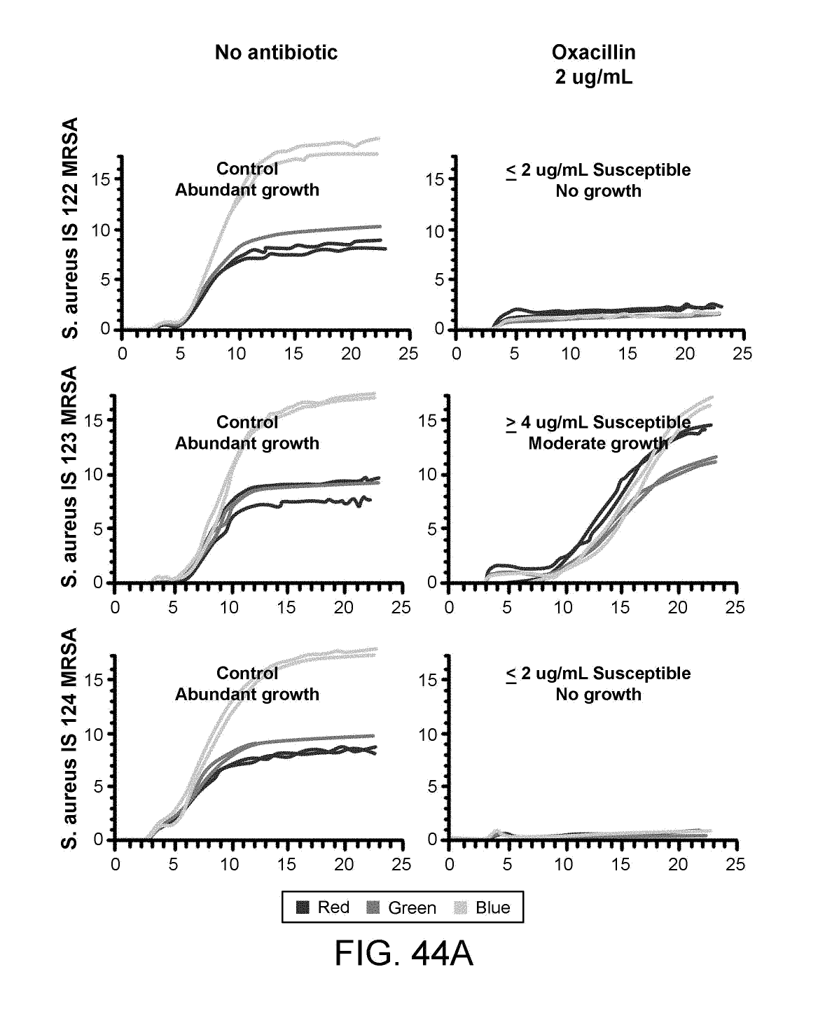

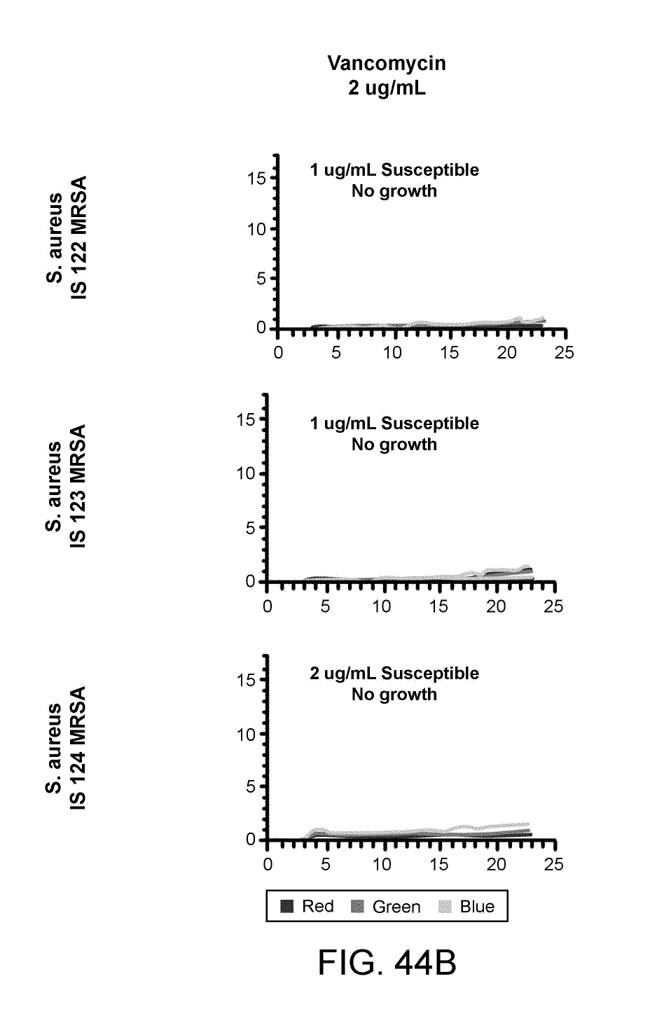

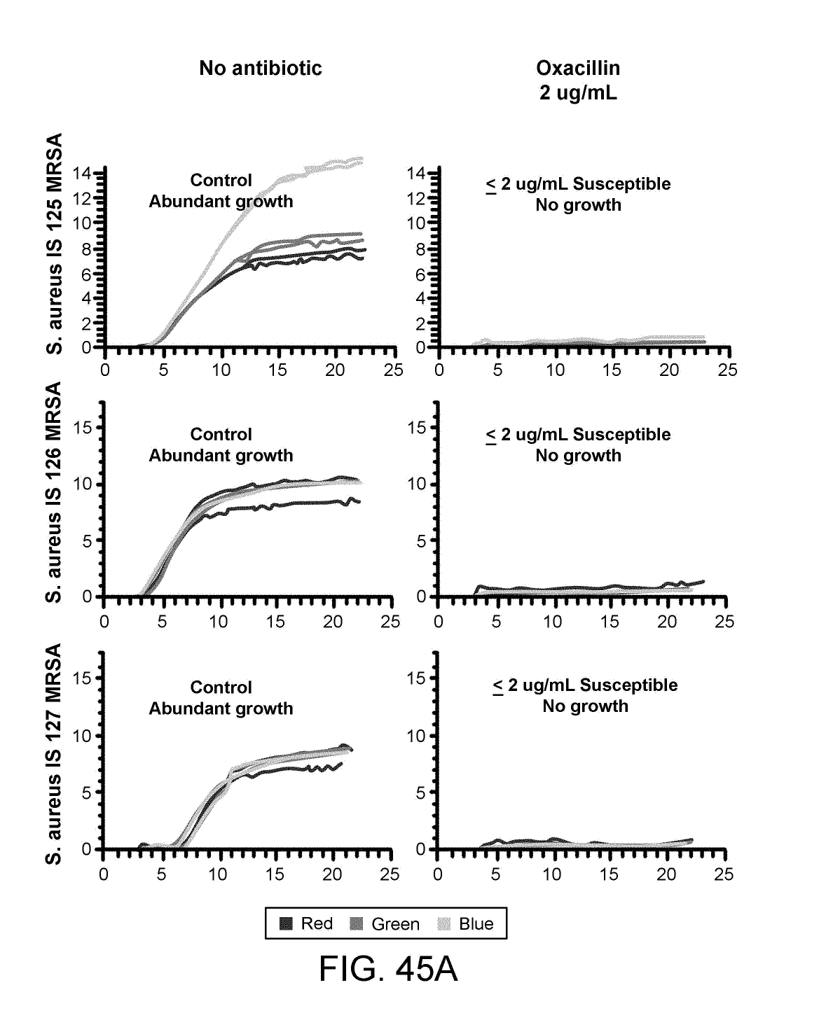

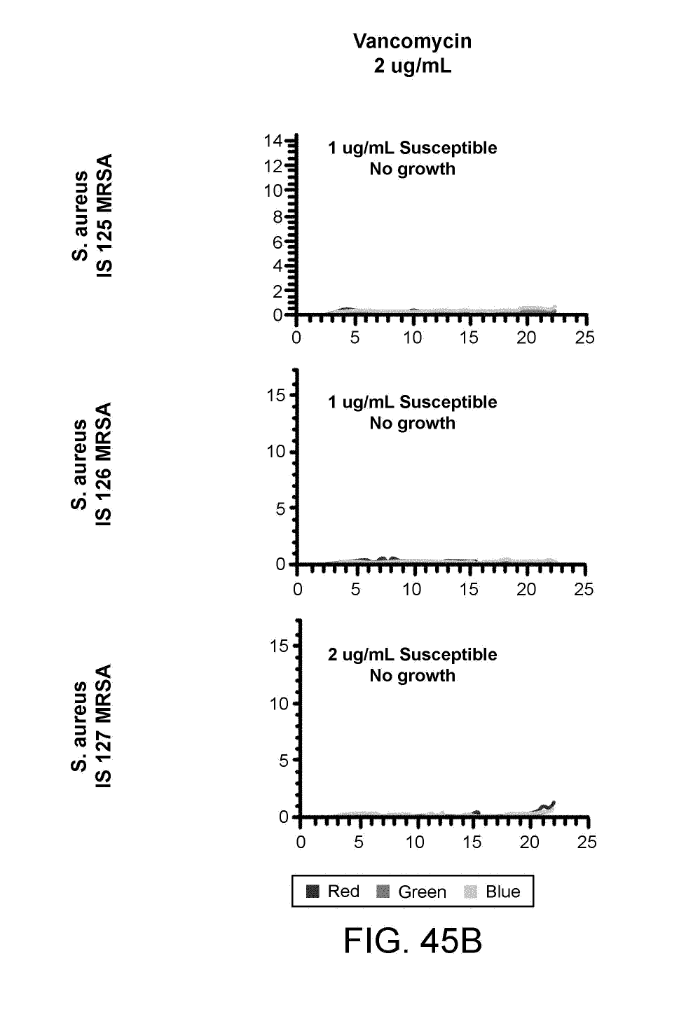

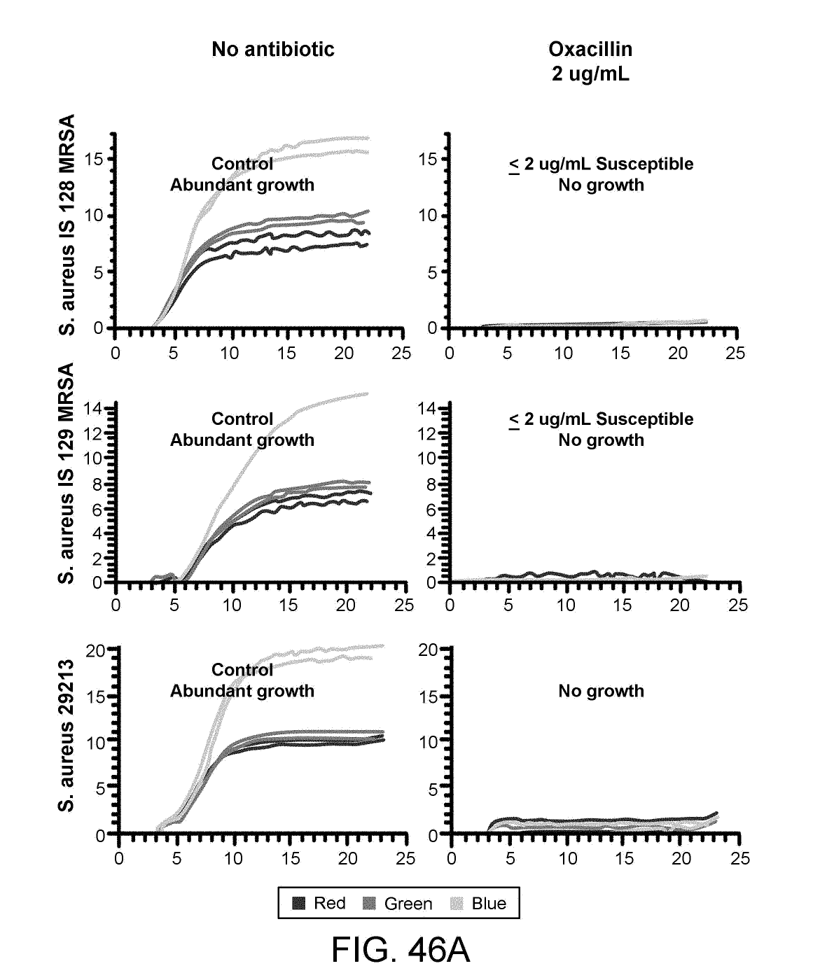

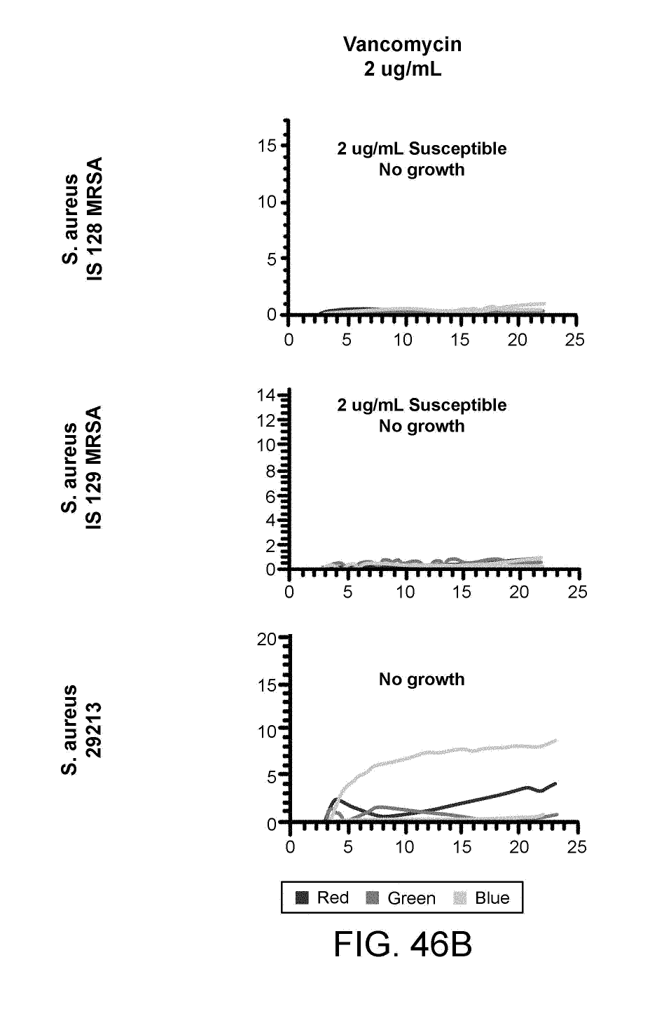

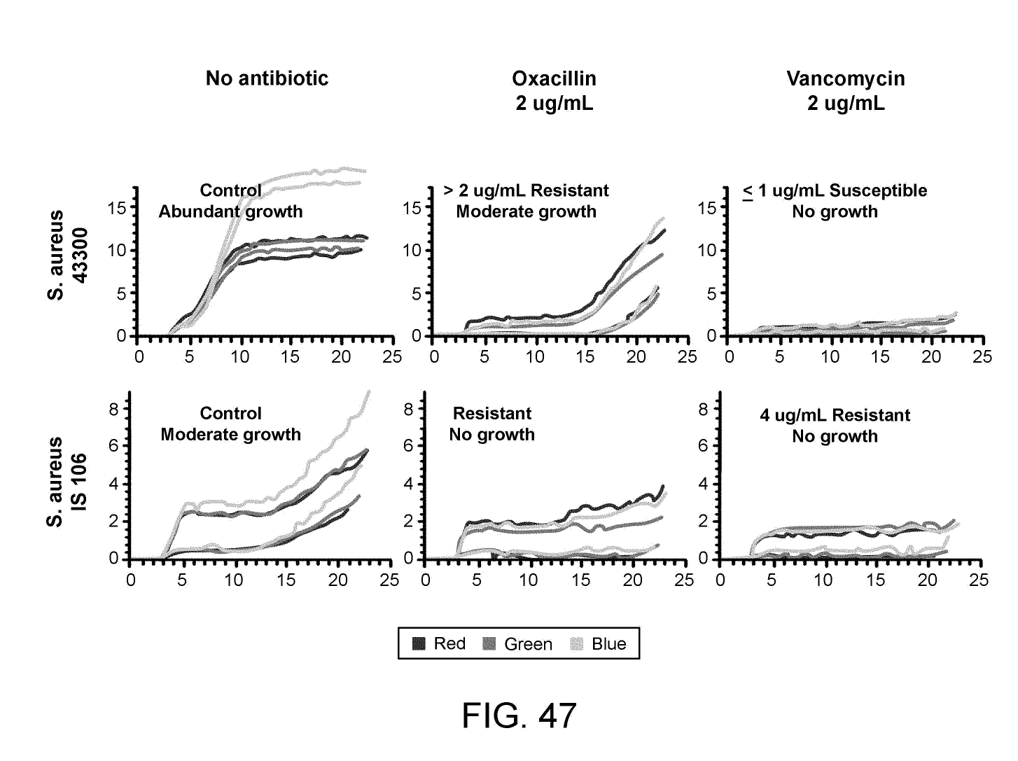

FIGS. 40A-47 show temporal results of susceptibility tests for various sensors of a colorimetric sensor array for identified strains of S. aureus.

From over 900 susceptibility tests with 98 different bacterial strains, it has been demonstrated that strain-specific susceptibility to antibiotic therapy can be achieved in a range of 6 to 8 hours. Thus, together with strain-specific identification within 8 to 16 hours, strains can be identified and susceptibility can be assayed in 28 hours or less, typically 24 hours or less.

### Susceptibility by Detecting Perturbation of VOCs or Other Compounds

In some embodiments, the susceptibility of microorganisms may be tested by changes in the sensor 808 response, rather than just whether or not the sensor 808 detects any response. For instance, disclosed are methods for determining whether a microorganism grows or dies in the presence of an antibiotic or other substance. This requires detection or absence of a sensor response in general, that indicates that the microorganism is growing, not growing or dying. This test, like the turbidity or carbon dioxide tests, only determine whether or not organisms may grow and requires a relatively longer span of time to determine whether organisms are growing. This is because these methods require enough time for the organisms to die or to multiply.

However, it has been discovered that detection of changes in VOC output of microorganism—in addition to changes in the number of microorganisms—can be detected by the sensor 808 after application of antibiotics. The change in the sensor 808 response can also be correlated to susceptibility of the microorganism to antibiotics. For instance, it has been discovered that after application of antibiotics, certain sensors decrease or show less response at certain concentrations. This change in VOC output can be correlated to the resistance or susceptibility of the microorganisms in the sample to the antibiotic or other substance introduced. In other examples, a change in compound output that remain in solution (not limited to VOCs) may be correlated to a susceptibility of the microorganisms.

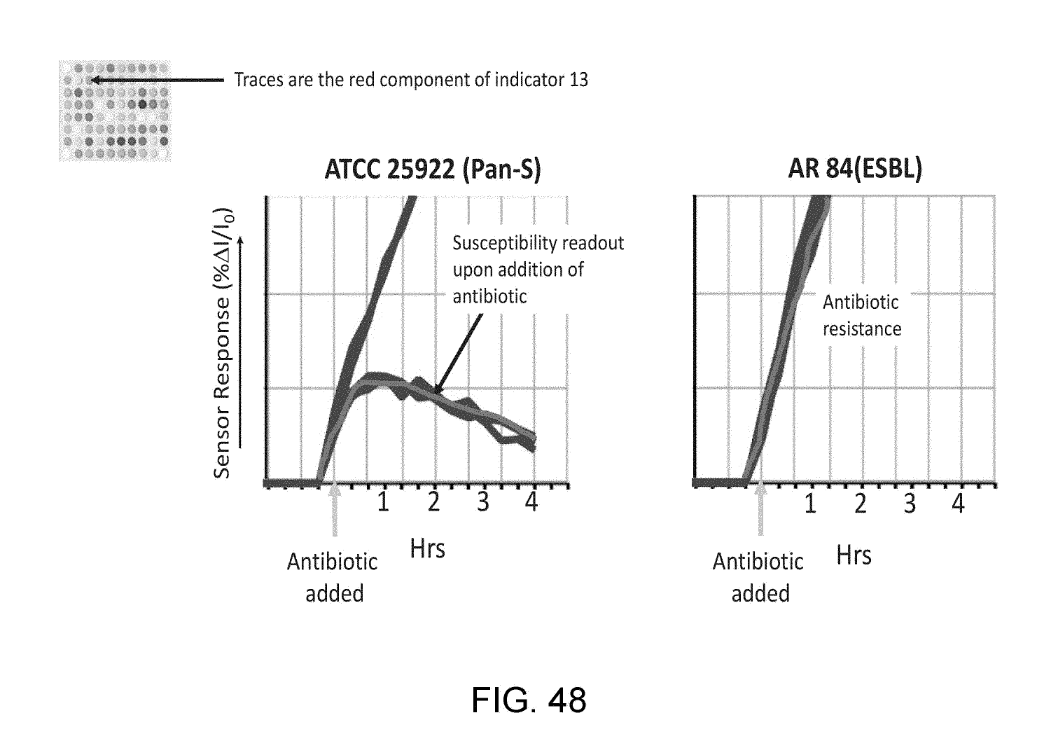

For instance, FIG. 48 illustrates an example of the response of the red spectrum of a single indicator (e.g. an indicator containing ZnTPP and Bromophenol Blue) on the sensor 808 in the presence of a sample. As shown, the antibiotic is added at 0.5 hours—in the example of the susceptible microorganism ATCC 25922 (Pan-S), the indicator red component drops off dramatically. The results can be noticed almost a half an hour to an hour after adding the antibiotic—far faster than to notice a general decline in emission of VOCs due to cell death or lack of growth of microorganisms.

In the resistant strain illustrated in FIG. 48, AR 84 (ESBL), the antibiotic is added at the same time, but the red component of the sensor response continues to increase over time. Accordingly, the change in intensity or lack of change in one indicator or sensor 808, may be indicative of whether the organism is susceptible to that particular antibiotic. Accordingly, in this embodiment, the sensor 808 response of a single indicator may be monitored. Changes in the slope of the indicator response over time, or decreases in the indicator may be monitored to determine when there is potentially a susceptible organism.

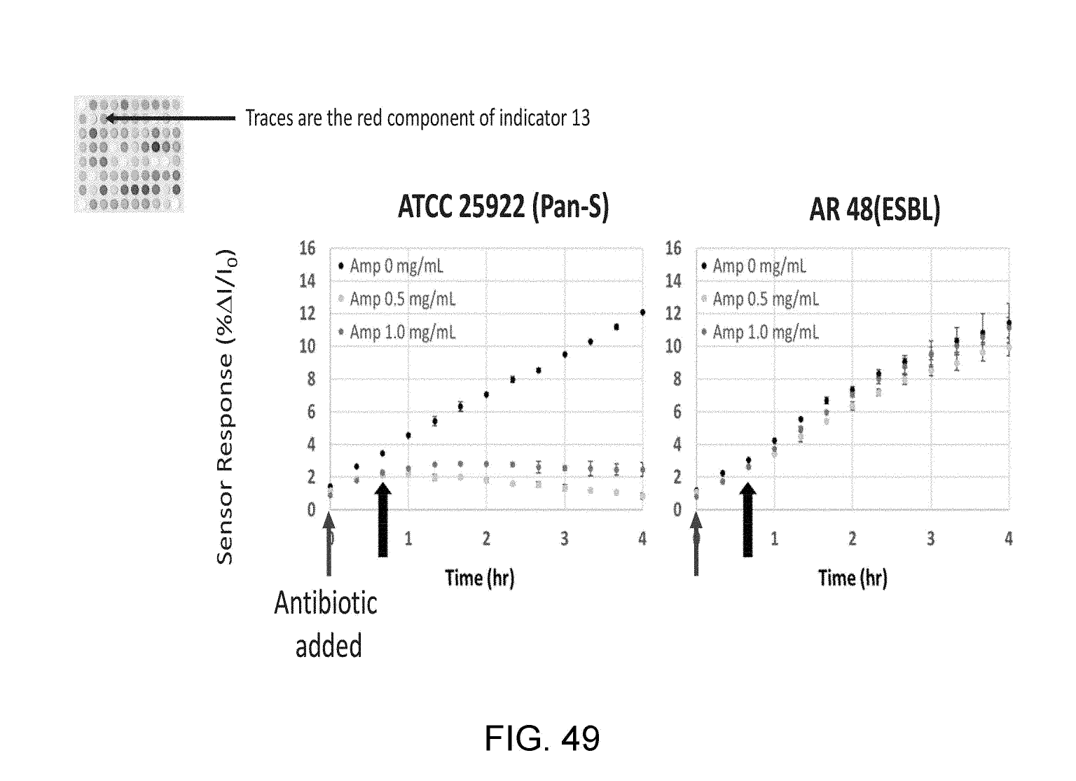

FIG. 49 illustrates the same two microorganisms where different concentrations of Ampicillin are added at the same time. As indicated, the susceptible strain shows the same response at 0.5 mg/ml and 1.0 mg/ml, and at all concentrations the resistant microorganism shows a decline in sensor response. Accordingly, the same indicator or sensor 808 consistently predicts Ampicillin resistance.

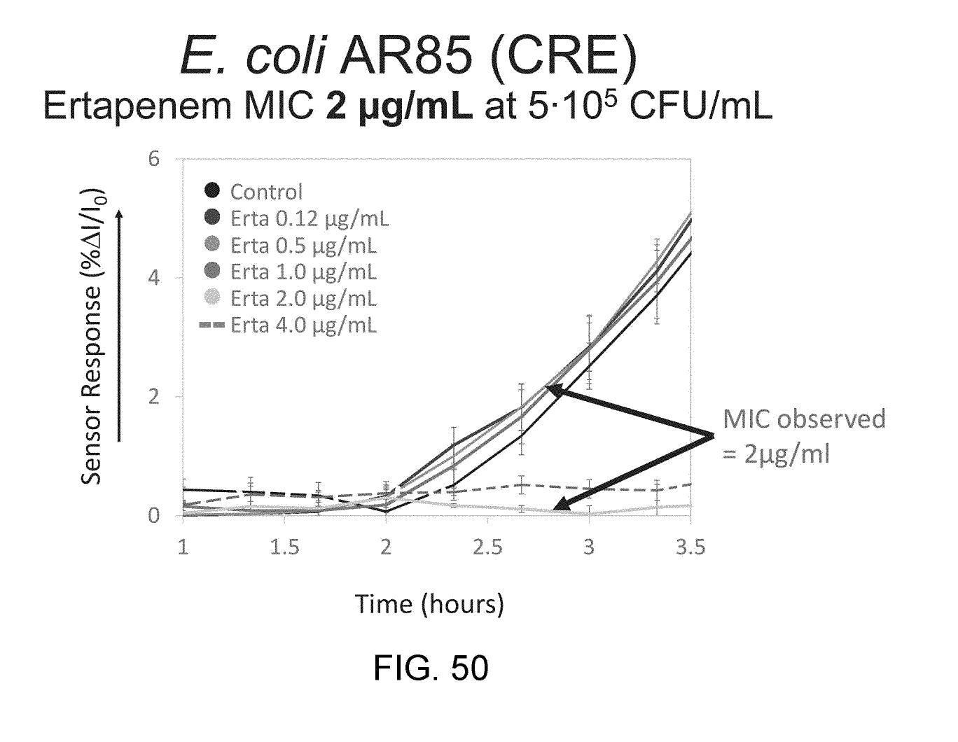

FIG. 50 illustrates a graph showing the sensor response of a single sensor 808, dye or indicator to a sample of E. coli incubated in the presence of different concentrations of Ertapenem—a Carbapenem family of antibiotic. As illustrated, the single indicator can not only determine whether this strain of E. coli (AR85) is resistant to Ertapenem, but also the minimum inhibitory concentration (“MIC”). As illustrated, the minimum inhibitory concentration of 2 g/ml is observed only after 2.5 hours. This is extraordinarily fast, and provides an excellent and fast method of determining how to take action if this sample is from a patient.

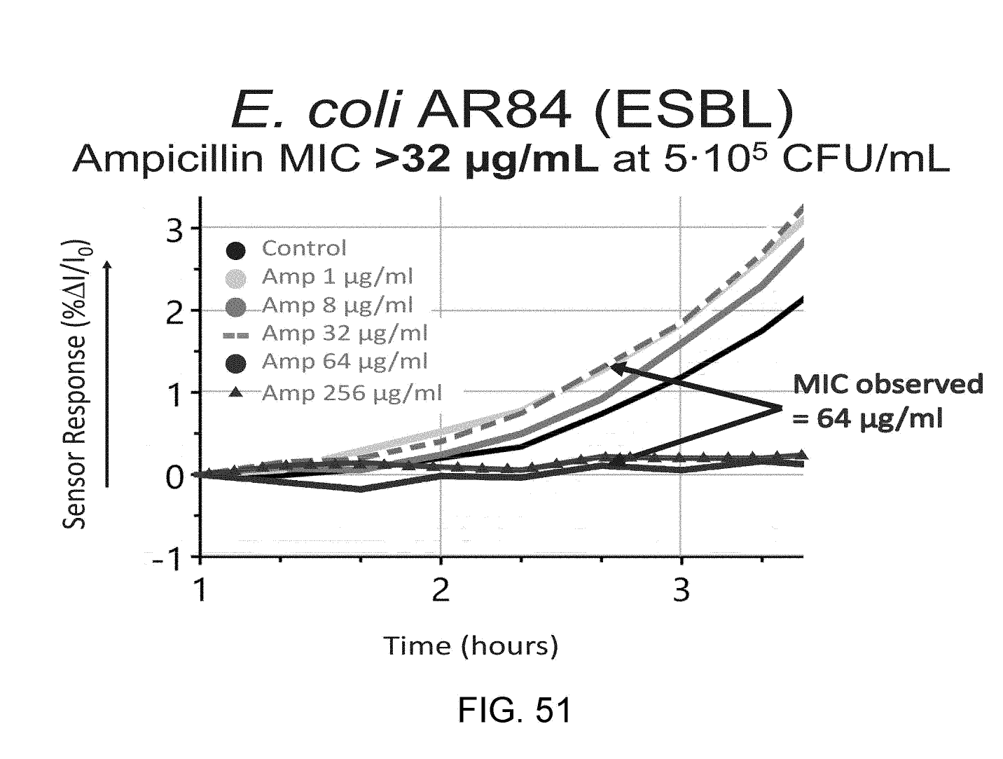

FIG. 51 illustrates a graph showing the MIC of Ampicillin observed for E. coli (AR84). As illustrated, the MIC observed for this strain in Ampicillin is 64 μg/ml at 5×10⁵ colony forming units (“CFU”)/ml. In this example, the MIC is observed in under three hours.

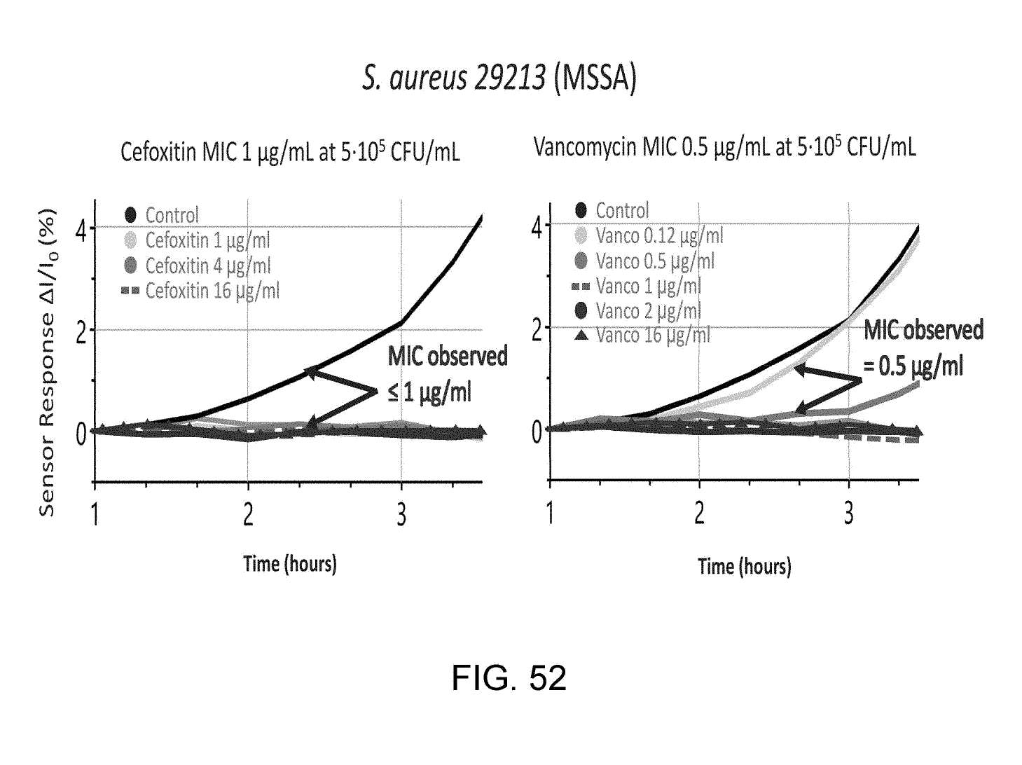

FIG. 52 illustrates two graphs showing the MIC observed for two different antibiotics to S. aureus 29213 (MSSA). The graph on the left portion of FIG. 52 illustrates a MIC of 1 μg/ml to Cefoxitin and the graph on the right illustrations a MIC of 0.5 μg/ml of Vancomycin. In this example, the MIC is observed in about 2.5 hours.

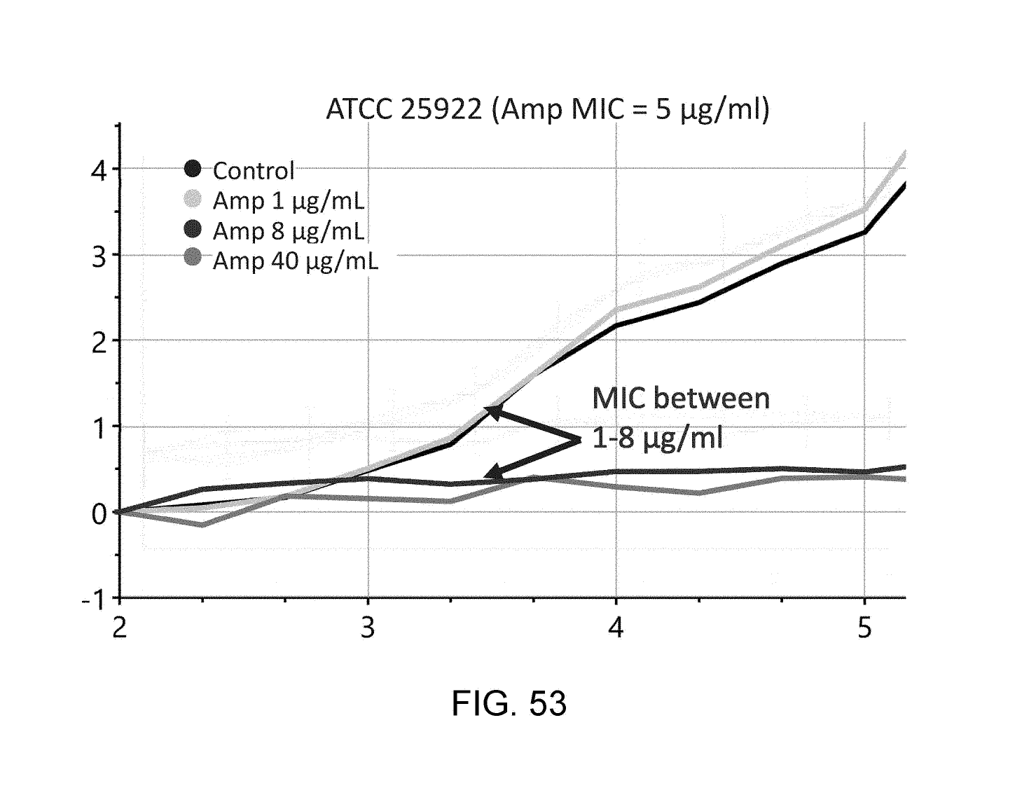

FIG. 53 illustrates a graph showing the MIC observed for ATCC 25922 for Ampicillin. As in the other examples, the sensor response for a single sensor 808 or indicator is sufficient to identify a MIC in a range of 1-8 μg/ml.

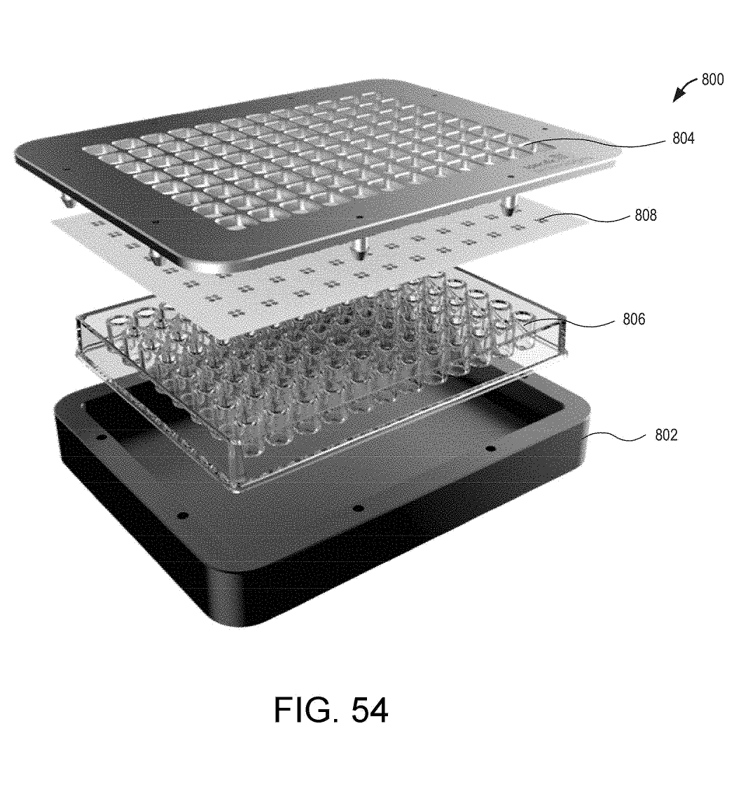

FIG. 54 illustrates a perspective view of an example container 808 configured for use to assess a susceptibility or resistance of microorganisms. Container 800 includes base 802 and lid 804, with wells (or plates) 806 positioned in base 802 opposite colorimetric sensor arrays 808 on lid 804. In this example, colorimetric sensor arrays 808 are printed on paper in an arrangement that lines up with the wells 806. Additionally, the lid includes ridges that form a seal around each of the colorimetric sensor arrays 808 and wells 806 to prevent cross contamination.

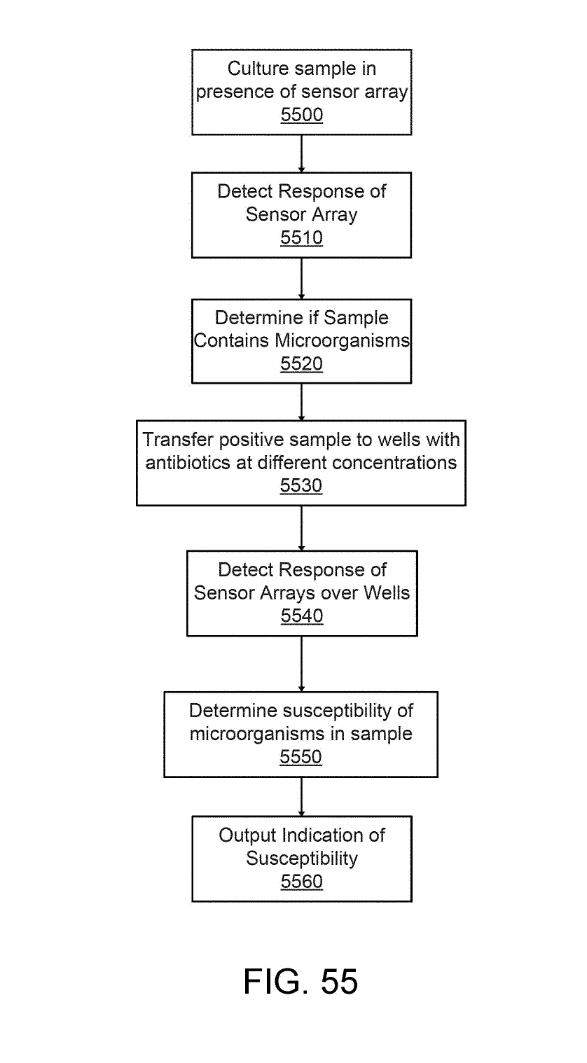

FIG. 55 illustrates a method of detecting susceptibility by detect perturbation of VOCs. For instance, in step 5500, a sample is taken from a patient and cultured in the presence of a sensor array 808, or other device to indicate whether live organisms are growing. After culturing the sample, the response of the sensor array will be continually monitored 5510 for a threshold response that indicates microorganisms are producing volatile organic compounds 5520. In some examples the sensor array 808 will detect microorganisms growing 5520 within 10 hours of culturing the sample. In some cases, this will be indicated by a threshold level of response or change in color of one or more of the sensors or dyes on the colorimetric sensor array 808.

Then in some examples, if the sample is determined to have microorganisms, then the sample may be transferred or divided into several samples or wells with antibiotics at different concentrations 5530. For instance, this may be accomplished by automatically or manually transferring portions of the sample to new wells, each of the wells with an antibiotic at a certain, known concentration. In one or a few of the wells, there may be a sample grown without any additional antibiotics. In same examples, the sample will be transferred to 80 or 96 wells. For instance, there may be 20 antibiotics at 4 concentrations each, with each well containing one antibiotic at one concentration.

Then, a sensor array 808 over each well will detect the response of that individual well 5540 and will be sealed from the VOCs emitted from the other wells in the container 800. Accordingly, each well will be in gaseous communication with a single sensor array 808 and each sensor array will be sealed from all wells but one. Then, the system will detect the response of the sensor arrays over the wells 5540 as disclosed herein, and the response will be processed to determine the susceptibility of any microorganisms in the sample 5550 to the given antibiotic at the given concentration. Then, the susceptibility results may be output 5560.

In some examples, the aggregate sensor response from each of the sensors 808 over each of the wells will be analyzed to determine a minimum inhibitory concentration for each of the antibiotics added to the wells. In some examples, this process will happen automatically as a detector scans each of the sensors, and determines the aggregate response.

In some examples, the change in sensor response will be calculated based on a difference between the sample cultured in a well that does not include an antibiotic and one where the sample is introduced into a well or other vial with an antibiotic. Accordingly, in this example, the system can process the different sensor responses 5540, to determine the difference between the change of sensor 808, for example one or more indicators on a sensor, between the wells with no antibiotics and the wells where antibiotics are added. In these examples, a threshold difference may determine a susceptibility or lack of susceptibility if the difference in the change in certain indicators does not cross a threshold.

In some examples, the sensor responses 5540 over the wells may be compared to previously recorded or average sensor responses for the same species and strain. Accordingly, if one of the wells contains the sample without antibiotics, either prior to or after dividing the sample into wells the species and/or strain can be identified. Accordingly, historical data of sensor response for certain indicators may be compared to the sensor response over each of the wells of samples cultured with antibiotics. In other examples, the sensor response of the non-antibiotic wells will be compared to the antibiotics wells to determine a susceptibility 5550 of known organisms with or without historical data on susceptibility, or unknown organisms to assess a phenotypic susceptibility 5550. Then, an indication of the susceptibility may be output 5560, to a display, or saved as a data file associated with a patient ID in some examples.

In some examples, threshold differences will be measured by sensor response changes at different time points or the same time points. In other examples, the trend of the sensor responses will be examined to extrapolate sensor responses. Accordingly, the slope or trend of certain indicators (including certain spectral filters, for example a red portion of a particular indicator) may indicate whether or not the organism is susceptible.

### Selected Embodiments

Although the above description and the attached claims disclose a number of embodiments of the present invention, other alternative aspects of the invention are disclosed in the following further embodiments.

A first possible embodiment is a method which starts with culturing a sample that includes microorganisms. The sample can be cultured in a growth medium that includes at least one test substance in communication with a colorimetric sensor array. The communication exposes sensors in the colorimetric sensor array to compounds emitted by at least some of the microorganisms. The method then continues by assessing the susceptibility of at least some of the microorganisms to the test substance. The assessment can be determined based on a response of the sensors in the colorimetric sensor array to the compounds produced by the microorganisms.

This embodiment can further comprise assessing a mode of resistance of the microorganism to the test substance based on response of the sensors in the colorimetric sensor array to the compounds produced by the microorganisms. The mode of resistance can be an efflux pump. Additionally, or alternatively, the mode of resistance can comprise one or more of: cell wall synthesis related mechanics, protein synthesis related mechanisms, nucleic acid replication related mechanisms, or cell wall porin related mechanisms. Additionally, or alternatively, the mode of resistance can be an enzymatic breakdown of the test substance, an alteration of a site to which the test substance binds, an alteration of a metabolic pathway, and/or a modification to a cell envelop of the microorganisms.

In some examples of the first embodiment, the susceptibility can be a partial susceptibility or level of susceptibility. The susceptibility can indicate a degree of susceptibility of the microorganisms to the test substance.

In some examples of the first embodiment, separate portions of the sample can be cultured with different concentrations of the test substance. The susceptibility can be separately assessed for each concentration of the test substance. The susceptibility of the microorganisms to the test substance within 48 hours, within 36 hours, within 24 hours, within 12 hours, within 10 hours within 8 hours, within 6 hours, 4 hours, within 2 hours, within 1 hour, or within 30 minutes after detection of the presence of the microorganisms by a growth detection system. The assessed susceptibility can be output to a caregiver as a numeric value. Thus numeric value can be calculated based on an amount of time it took to determine the susceptibility, a level of sensor response, and a concentration of the test substance utilized.

Based on the assessed susceptibilities to the test substance, a minimum inhibitory concentration of the test substance can be determined for the microorganisms.

Assessing the susceptibility can further comprise assessing a turbidity of the sample. The turbidity can be assessed using an optical detector that is also used to measure the response of the sensors.

The test substance can be a medication approved for human use.

The embodiment can comprise additional steps such as collecting the microorganism from a substrate before culturing the microorganisms. The substrate can be selected from at least one of: woven or nonwoven fabric, paper, metal, and plastic.

The embodiment can include additional steps such as collecting the microorganisms from a mammal before culturing the microorganisms. The mammal can be a human. Collecting the microorganisms from the mammal can comprise collecting a sample from the mammal, wherein the sample comprises a gas, solid, liquid, or a combination thereof. The sample can be blood, a dilution of microorganisms from a colony or other sample, sputum, nasal sample, rectal sample, microbiome sample, or other sample commonly produced in clinical microbiology laboratories. Alternatively, or in addition, the sample can comprise exhaled mammalian breath.

The first embodiment can include additional steps such as identifying at least a second test substance to which the microorganisms are susceptible based on assessed susceptibility of the microorganism to the test substance, wherein the second test substance is a medication approved for human and/or animal use. The embodiment can then further include administering a dose of the at least second test substance to the mammal from which the microorganisms were collected. The dose can be effective to reduce a population of the identified microorganisms in the mammal.

A second embodiment can be another method comprising culturing a sample that can contain microorganisms. The sample can be cultured in a medium which is in communication with a colorimetric sensor array. Sensors in the colorimetric sensor array can be exposed to compounds produced by the microorganism. The method can detect a response of the colorimetric sensor array to the compounds produced by the microorganism. The method can further determine a susceptibility of the microorganisms to a substance. The susceptibility can be determined by comparing a detected response of sensors on the colorimetric sensor array to a dataset of responses associated with known susceptibilities.

In some examples of the second embodiment, the dataset can include known strains of microorganisms associated with the known susceptibilities.

In some examples of the second embodiment, the sample can be cultured while exposed to an antibiotic.

In a third embodiment, the present disclosure can provide a method of reducing a microorganism population in a mammal showing symptoms of infection. The method can comprise culturing a sample that can contain microorganisms. The sample can be cultured in a medium that includes a first substance and the sample can further be in gaseous communication with a colorimetric sensor array. Sensors on the colorimetric sensor array can thereby be exposed to volatile organic compounds produced by the microorganism. The method can then determine a susceptibility of the microorganisms to the first substance based on a response of the sensors in the colorimetric sensor array to the volatile organic compounds produced by the microorganisms. The method can additionally identify a second substance to which the microorganisms are susceptible. The second substance can be identified based at least partially on the determined susceptibility of the microorganisms to the first substance. The method can then administer a dose of the second substance to the mammal, wherein the dose is effective to reduce the microorganism population in the mammal.

In some examples of the third embodiment, the method can include identifying the microorganisms by species and strain based on the response of the sensors in the colorimetric sensor array to the volatile organic compounds produced by the microorganisms before identifying the susceptibility of the microorganisms to the first substance. The first substance can be selected based on an identified species and strain of the microorganisms. The second substance can also be selected based on an identified species and strain of the microorganisms.

In other examples of the third embodiment, collecting the sample can include collecting a sample from the mammal, wherein the sample comprises a gas, solid, liquid, or a combination thereof. The sample can be blood, a dilution of microorganisms from a colony or other sample, sputum, nasal sample, rectal sample, microbiome sample, or other sample commonly collected in clinical microbiology laboratories. Alternatively, or in addition, the sample can comprise exhaled mammalian breath. The mammal can be a human and can be showing symptoms of a blood infection from microorganism. The microorganism causing the blood infection can be one or more of bacteria, fungi, archaea, protozoa, or algae.

In a fourth embodiment, the present disclosure can provide a method which separately cultures a plurality of portions of a sample. The sample can contain a species of a microorganism. Each portion can be separately cultured with one of a plurality of substances in a medium. Each separately cultured portion can be in gaseous communication with a separate colorimetric sensor array. Thereby, sensors in the colorimetric sensor array can be exposed to volatile organic compounds produced by the microorganisms. The method can then determine a susceptibility of the microorganisms to each of the plurality of substances based on response of the sensors in the colorimetric sensor arrays to the volatile organic compounds produced by the microorganisms.

In some examples of the fourth embodiment, at least one of the plurality of substances is identified as a substance to which the microorganism is susceptible.

In other examples of the fourth embodiment, an additional portion of the sample can be separately cultured without exposure to a substance.

In other examples of the fourth embodiment, culturing the microorganism can comprise culturing the microorganisms on a solid medium or in a liquid medium.

In other examples of the fourth embodiment, the sample can comprise microorganisms removed from a culture of mammalian specimen wherein the mammalian specimen comprises a gas, solid, liquid, or a combination thereof. The sample can be tissue taken directly from a mammal.

In other examples of the fourth embodiment, the response of each sensor can comprise a change in one or more color components of the sensor. A temporal and/or static response of each sensor can yield a temporal or static color response pattern of the microorganisms. Determining the susceptibility can further comprise comparing a temporal and/or static color pattern of the sensor with a library of temporal and/or static color response patterns. These patterns can be characteristic of known susceptibilities of microorganisms when exposed to antibiotics at known concentrations.

In other examples of the fourth embodiment, the susceptibility of the microorganisms to the substance can be assessed within 64 hours, within 48 hours, within 36 hours, within 24 hours, within 12 hours, within 10 hours, within 8 hours, within 4 hours, or within 2 hours after identification of the microorganisms by species and strain.

In other examples of the fourth embodiment, one of the plurality of substances can be is a medication approved for human use.

In other examples of the fourth embodiment, the method can include collecting the microorganisms from a substrate before culturing the microorganisms. The substrate can be selected from at least one of: woven or nonwoven fabric, paper, metal, and plastic.

In other examples of the fourth embodiment, the method can include collecting the microorganisms from a mammal before culturing the microorganisms. The mammal can be a human. Collecting the microorganisms from the mammal can comprise collecting a sample from the mammal, wherein the sample comprises a gas, solid, liquid, or a combination thereof.

A fifth embodiment of the present disclosure can provide a method for assessing the susceptibility of the microorganism to a substance. The method can include receiving colorimetric matrix information based upon exposure of sensors in a colorimetric sensor array to volatile organic compounds produced by microorganisms. The microorganisms can be cultured in a sample comprising the microorganisms and a substance in a medium. The method can further include assessing a susceptibility of the microorganisms to the substance based on the colorimetric matrix information and a response of sensors in a second colorimetric sensor array to the volatile organic compounds produced by the microorganisms.

In some examples of the fifth embodiment, the colorimetric matric information can be received for a plurality of portions of the sample. Each portion of the sample can be separately cultured with a different type or concentration of a substance. Then, the susceptibility of the microorganisms to each type or concentration of substance can be separately assessed. A substance to which the microorganism is susceptible can be identified based on assessed susceptibilities of the microorganism to each type or concentration of substance.

In other examples of the fifth embodiment, each portion of the sample can be cultured in a separate well of a test plate.

In other examples of the fifth embodiment, the colorimetric matrix information can be sent to a remote server, where the colorimetric matrix information is compared to data contained in a library. Alternatively, the colorimetric matrix information can be compared to data contained in a local library. The library, whether remote or local, can contain datasets with colorimetric matrix information associated with either known microorganism strains or known susceptibilities.

In other examples of the fifth embodiment, the substance can be a specific antibiotic. Assessing the susceptibility of the microorganism to the substance can comprise determining a susceptibility level of the microorganism to the specific antibiotic.

In other examples of the fifth embodiment, the method can include assessing a mode of resistance of the microorganism to the substance.

In other examples of the fifth embodiment, the susceptibility can be a degree of susceptibility and/or a partial susceptibility. The susceptibility can indicate a degree of susceptibility of the microorganism to the substance. The susceptibility can be assessed within 64 hours, within 48 hours, within 36 hours, within 24 hours, within 12 hours, within 10 hours, within 8 hours, within 4 hours, or within 2 hours after identification of the microorganisms. The susceptibility can be further output to a caregiver as a numeric value. The numeric value can be calculated based on an amount of time it took to determine the susceptibility, a level of sensor response, and a concentration of the substance utilized.

In other examples of the fifth embodiment, the substance can be a medication approved for human use.

In other examples of the fifth embodiment, the mode of resistance can be an efflux pump, an enzymatic breakdown of the substance, an alteration of a site to which the substance binds, an alteration of a metabolic pathway, and or a modification to a cell envelop of the microorganisms.

A sixth embodiment of the present disclosure can provide a method which cultures a sample in a medium. The medium can be exposed to a colorimetric sensor array. The method can determine whether the sample contains microorganisms based on a response of at least a subset of sensors in the colorimetric sensor array to volatile organic compounds produced by microorganisms. If the sample contains microorganisms, the method can introduce a substance to the sample. The method can assess a susceptibility of the microorganisms to the substance based on a change in at least a second subset of the sensors in the colorimetric sensor array to the volatile organic compounds produced by the microorganisms after addition of the substance.

In some examples of the sixth embodiment, introducing a substance to the sample if the sample contains microorganisms can further comprise dividing the sample into sub-samples and introducing different concentrations of the substance to each of the sub-samples.

In other examples of the sixth embodiment, assessing a susceptibility can include determining a minimum inhibitory concentration of the substance for the microorganisms. The susceptibility can be assessed within 1 to 3 hours after introducing the substance.

In other examples of the sixth embodiment, the microorganisms can be bacteria.

In other examples of the sixth embodiment, a change in at least the second subset of the sensors can be a change in intensity of at least one spectral frequency of at least one sensor. The at least one spectral frequency can be at least one of red, green, and blue. The change in intensity can be a rate of change in intensity. The change in intensity can be a threshold change in intensity.

A seventh embodiment of the present disclosure can provide a method for culturing a sample that contains microorganisms. The sample can be in a medium exposed to a substance and a colorimetric sensor array. The method can introduce a substance to the sample. The method can proceed to assess a susceptibility of the microorganisms to the substance. The susceptibility can be based on a change of at least one sensor in the colorimetric sensor array to volatile organic compounds produced by the microorganisms after addition of the substance.

In some examples of the seventh embodiment, introducing the substance to the sample can comprise introducing different concentrations of at least two substances to separate portions of the sample. Each portion can be exposed to separate colorimetric sensor arrays.

In other examples of the seventh embodiment, assessing the susceptibility can comprise determining a minimum inhibitory concentration of the at least two substances for the microorganisms.

In other examples of the seventh embodiment, the separate colorimetric sensor arrays can be printed on a single sheet.

In other examples of the seventh embodiment, at least one of the sensors can comprise at least one of ZNTPP and Bromophenol Blue.

In other examples of the seventh embodiment, at least one of the sensors can comprise at least one of a metalloporphyrin and a pH indicator.

An eighth embodiment of the present disclosure can provide a method for culturing a first portion and a second portion of a sample. The sample can comprise microorganisms. The first and second portion can be exposed to a first and second colorimetric sensor array. Sensors on the first and second colorimetric sensor array can thereby be exposed to volatile organic compounds produced by the microorganisms. The first portion of the sample can be cultured in a first enclosure with the first colorimetric sensor array with an antibiotic. The second portion of the sample can be cultured in a second enclosure with the second colorimetric sensor array without the antibiotic. The method can then include determining the identity of the microorganisms based upon the response of the sensors to the second colorimetric sensor array. The method can then determine the impact of the antibiotic on the microorganisms based upon the response of the sensors to the first colorimetric sensor array.

Implementations of the subject matter and the operations described in this specification can be implemented in digital electronic circuitry, or in computer software, firmware, or hardware, including the structures disclosed in this specification and their structural equivalents, or in combinations of one or more of them. Implementations of the subject matter described in this specification can be implemented as one or more computer programs, i.e., one or more modules of computer program instructions, encoded on computer storage medium for execution by, or to control the operation of, data processing apparatus. Alternatively, or in addition, the program instructions can be encoded on an artificially generated propagated signal, e.g., a machine-generated electrical, optical, or electromagnetic signal that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially generated propagated signal. The computer storage medium can also be, or be included in, one or more separate physical components or media (e.g., multiple CDs, disks, or other storage devices).

The data processing operations described in this specification can be implemented as operations performed by a data processing apparatus on data stored on one or more computer-readable storage devices or received from other sources.

The term “data processing apparatus” encompasses all kinds of apparatus, devices, and machines for processing data, including, by way of example, a programmable processor, a computer, a system on a chip, or multiple ones, or combinations of the foregoing. The apparatus can include special purpose logic circuitry, e.g., an FPGA (field programmable gate array) or an ASIC (application specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a standalone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA or an ASIC.

Processors suitable for the execution of any computer programs disclosed herein include, by way of example, both general and special purpose microprocessors, and any one or more processors of any kind of digital computer. Generally, a processor will receive instructions and data from a read-only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD-ROM and DVD-ROM disks. The processor and the memory can be supplemented by, or incorporated in, special purpose logic circuitry.

To provide for interaction with a user, certain implementations and/or portions of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's client device in response to requests received from the web browser.

Implementations of certain portions of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer to-peer networks).

Any computing systems disclosed herein can include clients and servers. A client and server are generally remote from each other and typically interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

While this specification contains many specific implementation details, these should not be construed as limitations on the scope of any inventions or of what may be claimed, but rather as descriptions of features specific to particular implementations of particular inventions. Certain features that are described in this specification in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations may be depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

Thus, particular implementations of the subject matter have been described. Other implementations are within the scope of the following claims. In some cases, the actions recited in the claims can be performed in a different order and still achieve desirable results. In addition, the processes depicted in the accompanying figures do not necessarily require the particular order shown, or sequential order, to achieve desirable results.

1. A method comprising:
culturing a sample that includes microorganisms, in a growth medium that includes at least one test substance in communication with a colorimetric sensor array, thereby exposing sensors in the colorimetric sensor array to compounds emitted by at least some of the microorganisms; and
assessing a susceptibility of at least some of the microorganisms to the test substance based on a response of the sensors in the colorimetric sensor array to the compounds produced by the microorganisms.

2. The method of claim 1, further comprising assessing a mode of resistance of the microorganism to the test substance based on the response of the sensors in the colorimetric sensor array to the compounds produced by the microorganisms.

3. The method of claim 1, wherein the susceptibility is a level of susceptibility.

4. The method of claim 3, wherein the level of susceptibility is a partial susceptibility.

5. The method of claim 1, wherein the susceptibility indicates a degree of susceptibility of the microorganisms to the test substance.

6. The method of claim 2, wherein the mode of resistance is an efflux pump.

7. The method of claim 2, wherein the mode of resistance comprises one or more of: cell wall synthesis related mechanics, protein synthesis related mechanisms, nucleic acid replication related mechanisms, and/or cell wall porin related mechanisms.

8. The method of claim 1, wherein separate portions of the sample are cultured with different concentrations of the test substance and the susceptibility is separately assessed for each concentration of the test substance.

9. The method of claim 8, wherein a minimum inhibitory concentration of the test substance is determined for the microorganisms based on assessed susceptibilities to the test substance.

10. The method of claim 1, wherein the susceptibility of the microorganisms to the test substance is assessed within 48 hours, within 36 hours, within 24 hours, within 12 hours, within 10 hours, within 8 hours, within 6 hours, 4 hours, within 2 hours, within 1 hour, or within 30 minutes after detecting presence of the microorganisms by a growth detection system.

11. The method of claim 1, wherein the susceptibility is output to a caregiver as a numeric value.

12. The method of claim 1, wherein assessing the susceptibility further comprises assessing a turbidity of the sample.

13. The method of claim 12, wherein the turbidity is assessed using an optical detector that is also used to measure the response of the sensors.

14. The method of claim 11, wherein the numeric value is calculated based on an amount of time it took to determine the susceptibility, a level of the sensor response, and a concentration of the test substance utilized.

15. The method of claim 1, wherein the test substance is a medication approved for human use.

16. The method of claim 2, wherein the mode of resistance is an enzymatic breakdown of the test substance.

17. The method of claim 2, wherein the mode of resistance is an alteration of a site to which the test substance binds.

18. The method of claim 2, wherein the mode of resistance is an alteration of a metabolic pathway.

19. The method of claim 2, wherein the mode of resistance is a modification to a cell envelop of the microorganisms.

20. The method of claim 1, further comprising collecting the microorganism from a substrate before culturing the microorganisms.

21. The method of claim 20, wherein the substrate is selected from at least one of: woven or nonwoven fabric, paper, metal, and/or plastic.

22. The method of claim 1, further comprising collecting the microorganisms from a mammal before culturing the microorganisms.

23. The method of claim 22, wherein the mammal is a human.

24. The method of claim 22, wherein collecting the microorganisms from the mammal comprises collecting a sample from the mammal, wherein the sample comprises a gas, solid, liquid, or a combination thereof.

25. The method of claim 24, wherein the sample comprises one or more of blood, a dilution of microorganisms from a colony or other sample, sputum, nasal sample, rectal sample, microbiome sample, or other sample commonly collected in clinical microbiology laboratories.

26. The method of claim 22, wherein the sample comprises exhaled mammalian breath.

27. The method of claim 1, further comprising identifying at least a second test substance to which the microorganisms are susceptible based on assessed susceptibility of the microorganism to the test substance, wherein the second test substance is a medication approved for animal and/or human use.

28. The method of claim 27, further comprising administering a dose of the at least second test substance to a mammal from which the microorganisms were collected, wherein the dose is effective to reduce a population of the microorganisms in the mammal.

29. A method comprising:
culturing a sample that may contain microorganisms in a medium that is in communication with a colorimetric sensor array, thereby exposing sensors in the colorimetric sensor array to compounds produced by the microorganism; and
detecting a response of the colorimetric sensor array to the compounds produced by the microorganism; and
determining a susceptibility of the microorganisms to a substance based on comparing the response of the sensors to a dataset of responses associated with known susceptibilities.

30. The method of claim 29, wherein the dataset includes known strains of microorganisms associated with the known susceptibilities.

31. The method of claim 29, wherein the sample is cultured while exposed to an antibiotic.
